(19) United States
(12) Patent Application Publication
(10) Pub. No.: US 2015/0307876 A1
(43) Pub. Date: Oct. 29, 2015

(54) DNA ASSIMILATION

(71) Applicant: REGENTS OF THE UNIVERSITY OF MINNESOTA, Minneapolis, MN (US)

(72) Inventor: Eric Hendrickson, Minneapolis, MN (US)

(21) Appl. No.: 14/377,462

(22) PCT Filed: Feb. 9, 2013
(86) PCT No.: PCT/US13/25460

§ 371 Date: Aug. 7, 2014

Related U.S. Application Data
(60) Provisional application No. 61/597,508, filed on Feb. 10, 2012.

(51) Int. Cl. C12N 15/113

(52) U.S. Cl. CPC C12N 15/113; C12N 2750/14143; C12N 2310/14

(57) ABSTRACT

Gene targeting is a valuable tool for basic researchers and gene therapists. Unfortunately, current methods utilised to target genes are inefficient because of their low targeting frequencies. Provided herein are methods and compositions by which gene targeting frequencies can be increased.

### RELATED APPLICATIONS

This application claims the benefit of U.S. Provisional Application No. 61/597,508, filed on Feb. 10, 2012, which is incorporated herein by reference in its entirety.

### STATEMENT OF GOVERNMENT RIGHTS

This invention was made with the assistance of government support under United States Grant Nos. 1RO1GM088351 from the National Institutes of Health. The government has certain rights in the invention.

### BACKGROUND OF THE INVENTION

Gene targeting is a valuable tool for basic researchers and gene therapists. Unfortunately, current methods utilized to target genes are inefficient because of their low targeting frequencies.

### SUMMARY OF THE INVENTION

An embodiment provides a method to increase gene targeting frequency comprising inhibiting expression of at least one gene of a mismatch repair pathway (MMR) or by inhibiting activity of at least one protein of a mismatch repair pathway so as to provide increased gene targeting frequency as compared to a cell in which expression and/or activity has not be inhibited.

An embodiment provides a method to increase gene targeting frequency comprising increasing expression of at least one gene coding for Rad52, Rad57, Rad59, MUS81, XRCC3 or a combination thereof so as to provide increased gene targeting frequency as compared to a cell in which expression has not been increased.

In one embodiment, the gene or protein is MLH1, PMS2, MSH2, MSH6, MSH3, PMS1, MLH3 or a combination thereof. In another embodiment, the expression is transiently inhibited. In one embodiment, the protein activity is inhibited by a small molecule or expression of the protein is inhibited by antisense, siRNA or shRNA.

In an embodiment, the DNA assimilation and/or targeting is mediated by a retrovirus, rAAV, dsDNA, ssDNA (e.g., a ssDNA oligo), zinc finger nuclease, homing nuclease, meganuclease, transcription activator like (TAL) effector nuclease or a combination thereof.

In one embodiment, the cell in which the mismatch repair gene or protein expression/activity is to be inhibited is mismatch repair proficient.

### BRIEF DESCRIPTION OF THE DRAWINGS

FIGS. 1i-vii: Presents a hypothetical pathway for the role of HR factors in gene targeting. The double line represents a transfected donor dsDNA that has homology to a preselected location within the recipient cell's genome and the hatched box represents a positive drug selection marker or a section of DNA containing the researcher's desired modification. (i) An unknown nuclease (PacMan™) resects the ends of the donor DNA. (ii) RPA (hatched oval circle) then coats the ssDNA ends. (iii) Rad51 (empty ellipse) and Rad52 (filled circle) then bind onto the ssDNA ends, displacing RPA in the process. (iv) The donor DNA complexed with Rad51 and Rad52 then associates with a chromosome (long double line hairpinned ends) containing homologous sequences (open box). (v) With the assistance of Rad54 (open circle with dot) and DNA replication, the resected ends invade the donor DNA and set up a double Holliday Junction. The donor DNA shown in (v) is rotated with respect to the donor DNA shown in (iv) for the sake of presentation. (vi) The repeated action of a resolvase (star) then completes the recombination process. (vii) At the end, the donor DNA has been integrated into a homologous region on an endogenous chromosome. In all panels, the vertical arrows are drawn implying a temporal order to each process, although in many cases, the precise sequence of events is not known and thus could be occurring in an order differently from what is shown or simultaneously, etc.

FIGS. 2i-vii: Two-ended, ends-out dsDNA gene targeting yields trans products of recombination. All symbols are as in FIG. 1 with the addition that * indicates a single nucleotide polymorphism (SNP) and the inverted > indicates a position of heteroduplex. The panels (i) through (iv) are as described in FIG. 1. It is, however, noted that due to the separate stand invasions (step v), the SNPs are transferred to the chromosome in a strand-specific fashion. The resolution of the resulting Holliday Junction generates an intermediate that contains heteroduplex at the sites of the SNPs (vii). When this intermediate is further resolved via DNA replication, two products are generated in which the SNPs have become stably transferred to the chromosome in a trans configuration (viii).

FIGS. 3i-vi: An example of a pathway for assimilation of single-stranded DNA during gene targeting is provided. A ssDNA is shown at top that has homology to a location within the recipient cell's genome. The hatched box represents a positive drug selection marker or a section of DNA containing the preselected modification and the asterisks (*) represents SNPs. The ends of the ssDNA are depicted as circles (their configuration inside cells is unknown). In the case of rAAV, the ends would be in the form of hairpinned inverted terminal repeats (ITRs). (i) The incoming ssDNA is likely coated with RPA (hatched oval circle). (ii) Rad59 (empty ellipse) and Rad52 (filled circle) can then bind onto the ssDNA, displacing the RPA. (iii) The donor ssDNA complexed with Rad59 and Rad52 can then associate with a chromosome (long double line with hairpinned ends) containing homologous sequences (open box). (iv) The ssDNA invades the donor DNA. (v) The action of a resolvase (star) can generate a recombination intermediate that contains heteroduplex at the sites of the homology and the SNPs. (vi) When this intermediate is further resolved via DNA replication, two products are generated and the SNPs have become stably transferred to the chromosome in a cis configuration. Only one of the recombinant products (the one also containing the SNPs) will contain the drug resistance marker. At the end, the donor DNA is integrated into a homologous region on an endogenous chromosome.

FIG. 4: Depicts the rAAV gene targeting vector used in studies at the HPRT locus. The shaded rectangles at either ends represent the viral ITRs. The open rectangles represent the left and right homology arms and the length of each is indicated. The filled rectangle represents the drug selection cassette, which for the majority of studies was puromycin (Puro). The positions of the restriction enzyme recognition sites and SNPs and distances (in bp) away from the drug selection cassette are indicated by the arrows. The positions of the palindromes are indicated by the arches.

FIGS. 5A-B: (A) A schematic showing the approach that was used to generate and then characterize rAAV-mediated correctly gene targeted clones at the HPRT locus. The HPRT NENASSXS+HP rAAV vector (FIG. 4) was converted to virus (i) that was then used to infect the target HCT116 cells in 6-well plates (ii). The cells were then placed under double drug selection (iii). G418 was used to select for the presence of the gene targeting cassette (although the exact drug varied from experiment to experiment) and 6-thioguanine was used to select for the loss of HPRT expression. The selections were carried out in 96-well plates (iv) and after approximately a month, individual clones were expanded and their DNA was characterized (v). (B) PCR and restriction enzyme analysis of doubly-drug resistant clones. Top: a depiction of the strategy for using PCR to analyze the left and right homology arms. Below: ethidium bromide-stained agarose gels showing representative results. The restriction enzymes used in the analyses are indicated on the left of each panel and each lane, in parallel, represents an individual clone. The white arrow indicates a clone that picked up the viral EcoRI, NcoI and SacII sites, but not the NdeII, XbaI or SbfI sites.

FIG. 6: A summary of the HPRT gene targeting experiments. The relevant restriction enzyme or palindromic sites are indicated at the top. The acquisition of a viral restriction enzyme site or palindromic sequence is indicated by a (+) and the absence of one by a (−). Clones in which sites occurred in cis are indicated in blue and those where they occurred in trans in yellow. The total number of independent clones corresponding to a specific configuration is denoted by “count” in the far right hand lane. A compilation of the frequency within the total population for a particular site being acquired is indicated.

FIG. 7: A summary of the SNP patterns observed for random rAAV gene targeting vector integration events. Independent clones that had integrated the HPRT NENASSXS+2HP vector at random locations were subjected to the PCR/restriction enzyme analysis outlined in FIG. 5. All of the clones (15/15) showed the complete acquisition of all the viral restriction enzyme sties (+).

FIG. 8: A summary of the HPRT gene targeting experiments in the parental HCT116 cell line addressing the retention of SNPs. The frequency with which a particular SNP site was retained in a correct HPRT gene targeting event (i.e., FIG. 6) is shown for the left (green triangles) and right (blue rectangles) homology arms. SNPs located near the drug resistance marker are highly retained whereas those far away are rarely retained. In addition, the pattern for SNP retention in the randomly targeted clones (i.e., FIG. 7) is similarly shown (dashed horizontal lines at top).

FIGS. 9A-C: A summary of the HPRT gene targeting experiments in the MLH1-corrected HCT116 cell line addressing the retention of SNPs. Panels A, B and C are comparable to FIGS. 6, 7 and 8, respectively and all symbols are as defined in those figures. Although the data sample is smaller for the MLH1-corrected HCT116 cell line, the overall patterns are similar to the parental (MMR-defective) HCT116 cell line.

FIG. 10: A summary of the relative gene targeting frequencies obtained in human cell lines defective for canonical HR genes. The cell lines are listed on the bottom: WT, wild-type; RAD54, Rad54B-null; XRCC3, XRCC3-null; MUS81, Mus81-null. The left panel shows relative gene targeting frequencies (in %) from experiments in which dsDNA was transfected into cells to obtain targeted clones (DNA Tx). These data were obtained from Miyagawa et al. (2002) and Yoshihara et al. (2004), and thus, there are 2 sets of data for RAD54 and XRCC3. The data in the right panel was derived from the instant rAAV-mediated gene targeting studies (rAAV). In all cases, each bar corresponds to the data obtained for a gene targeting study carried out at a particular locus, usually HPRT.

FIG. 11: The impact of MMR on rAAV-mediated gene targeting frequencies. A summary of the relative gene targeting frequencies obtained in either the parental (WT; MMR-defective) or the MLH1-complemented (+MLH1; MMR proficient) human cell lines. Two targeting vectors were utilized. One contained 15 individual mismatches to the target sequence (HPRT) and the other contained only 2 mismatches.

FIGS. 12A-E: The construction of a human RAD52-null cell line. (A) A schematic of the rAAV targeting vector used for inactivating RAD52. (B) A schematic for the RAD52 genomic locus and the approximate locations of relevant PCR primers. (C) A schematic of the RAD52 genomic locus following correct gene targeting. (D) A schematic of the RAD52 genomic locus following Cre-mediated removal of the NEO selection cassette. (E) A Western blot analysis of several resulting cell lines. RAD52 is shown, as is actin, as a loading control. +/+ indicates the parental cell line; +/− indicates a RAD52 heterozygous cell line; −/− indicates 4 independent RAD52-null cell lines.

FIGS. 13A-B: MSH2 knockdown increases rAAV-mediated gene targeting frequencies in the MMR-proficient MCF10a cell line. MCH10a, HCT116 and DLD-1 cells were transfected with siRNAs against MSH2, a scrambled control siRNA (ctrl) or left untreated (NT) and cultured for 48 hours. (A) Cells were then infected with rAAV vectors to target the BRAF V600E mutation and cultured under G418 selection for 2 weeks. DNA was harvested from the selected cells and the proportion of correctly targeted BRAF V600E alleles was determined by digital droplet PCR. The ratio of targeted to -non-targeted alleles for each treatment is expressed as fold change relative to the untreated control. Data shown is the average of duplicate samples; error bars represent standard deviation. (B) Western blot analysis of MSH2 protein in the cell lines 48-hours after transfection with 20 nm MSH2 siRNA or left untreated.

FIGS. 14A-G Gene targeting is marked by a characteristic SNP retention signature. (A and B) rAAV and dsDNA targeting vectors. The NEO selection cassette (white) is flanked by Has (green and blue), NdeI, EcoRI, NcoI, AseI, SSpI, SacI, SbaI and SbfI represent vector-specific restriction sites created by SNPs. LHP/RHP represent 22 bp vector-specific palindrome sequences created by the introduction of 3 SNPs. The flanking hairpin structures in (A) represent the viral ITRs. (C and D) The recipient HPRT locus before and after gene targeting. The NEO cassette replaces exon 3 of HPRT gene (grey) upon correct targeting. The corresponding positions of the viral Has and markers are indicated in bold lines and (?) symbols, respectively. Arrows represent PCR primer sites. P1:P3 and P4:P6 amplify the left and right Has of the GT clones, and P2:P3 and P4:P5 amplify the Has of the RI clones. The LHP destroys a chromosomal BbvCI site upon integration. (E, F and G) SNP retention signatures of rAAV targeting, random insertions and dsDNA targeting. The rAAV and dsDNA vectors are indicated in (A) and (D), respectively. The distance (D) to the central heterology is calculated from the inner ends of the homology arms. Markers on the left HA are indicated with the negative distances. Solid lines represent the linear regression between the retention frequency and the distance of the viral markers.

FIGS. 15A-F. rAAv-mediated gene targeting is suppressed in a MMR-proficient background. (A) The rAAV targeting vectors. All symbols are as in FIG. 15. 2 SNPs and 14 SNPs indicate the number of mismatches within the Has. (B) The effects of mismatches and the host MMR status on rAAV targeting efficiency. Targeting efficiency is expressed as GT/RI normalized to the wild-type. The mean+/−SEM of three independent experiments is shown. The MLH1 expression in the parental (wt) and MLH1⁺ cell lines is shown in the Western blot inset panel. (C and D) SNP retention signatures of rAAV targeting and random insertions in the MMR-proficient background. All symbols are as in FIG. 15E. (E and F) the MEPS model of recombination for homologous and homeologous sequences, respectively.

FIGS. 16-20 provide tables regarding SNP retention of rAAV GT clones in parental HCT116 (FIG. 16); SNP retention of plamid-based GT clones in parental HCT116 (FIG. 17); SNP rentention of rAAV RI clones in parental HCT116 (FIG. 18): SNP retention of rAAV GT clones MLH⁺ HCT116 (FIG. 19); and SNP retention of rAAV RI clones in MLH⁺ HCT116 (FIG. 20).

### DETAILED DESCRIPTION OF THE INVENTION

Using genetics (mutant cell lines), molecular biology (e.g., RNAi/shRNA) and biochemistry (chemical inhibitors), genes are identified that modulate gene targeting, such as viral (rAAV), ssDNA, dsDNA, meganuclease, TAL and Zn-finger mediated gene targeting. The present invention is generally directed, in part, towards methods, mechanisms, compositions, and kits for initiating, modulating, and/or stimulating homologous recombination. Simultaneously, the present invention improves targeted integrations by decreasing the randomness of undesired, non-targeted integrations. The methods of the invention provide elevated frequencies of correct gene targeting from, for example, viral-mediated gene targeting.

The invention may be used for any purpose including, for example, research, therapeutics, and generation of cell lines or transgenic animals (e.g., non-human animals such as mice, rats, guinea pigs, domestic animals, etc.). The cells and transgenic animals may be used in gene therapy or to study gene structure and function or biochemical processes. In addition, the transgenic mammals may be used as a source of cells, organs, or tissues, or to provide model systems for human disease.

### DEFINITIONS

As used herein, the terms below are defined by the following meanings:

“Host organism” is the term used for the organism in which gene targeting, according to the invention, is carried out. “Host cell” or “target cell” refers to a cell to be transduced/transfected with a specific viral vector/nucleic acid. The cell is optionally selected from in vitro cells such as those derived from cell culture, ex vivo cells, such as those derived from an organism, and in vivo cells, such as those in an organism. “Cells” include cells from, or the “subject” is, a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird. “Cell line” refers to individual cells, harvested cells and cultures containing cells. A cell line can be continuous, immortal or stable if the line remains viable over a prolonged period of time, such as about 6 months. “Cell line” can also include primary cell cultures. Cells which may be subjected to gene targeting may be any mammalian cells of interest, and include both primary cells and transformed cell lines, which may find use in cell therapy, research, interaction with other cells in vitro or the like.

“Target” refers to the gene or DNA segment or nucleic acid molecule, subject to modification by the gene targeting method of the present invention. Generally, the target is an endogenous gene, coding segment, control region, intron, exon, or portion thereof, of the host organism. The target can be any part or parts of genomic DNA.

“Target gene modifying sequence” is a DNA segment having sequence homology to the target, but differing from the target in certain ways, in particular, with respect to the specific desired modification(s) to be introduced in the target.

“Marker” is the term used herein to denote a gene or sequence whose presence or absence conveys a detectable phenotype of the organism. Various types of markers include, but are not limited to, selection markers, screening markers, and molecular markers. Selection markers are usually genes that can be expressed to convey a phenotype that makes the organism resistant or susceptible to a specific set of conditions. Screening markers convey a phenotype that is a readily observable and a distinguishable trait. Molecular markers are sequence features that can be uniquely identified by oligonucleotide or antibody probing, for example, RFLP (restriction fragment length polymorphism), SSR markers (simple sequence repeat), epitope tags and the like.

The term “isolated” refers to protein(s)/polypeptide(s), nucleic acid(s)/oligonucleotide(s), factor(s), cell or cells which are not associated with one or more protein(s)/polypeptide(s), nucleic acid(s)/oligonucleotide(s), factors, cells or one or more cellular components that are associated with the protein(s)/polypeptide(s), nucleic acid(s)/oligonucleotide(s), factor(s), cell or cells in vivo.

“Cells” include cells from, or the “subject” is, a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, and orangutan), rat, sheep, goat, cow and bird.

An “effective amount” generally means an amount that provides the desired local or systemic effect and/or performance.

As used herein, “fragments,” “analogues” or “derivatives” of the polypeptides/nucleotides described include those polypeptides/nucleotides in which one or more of the amino acid residues are substituted with a conserved or non-conserved amino acid residue and which may be natural or unnatural. In one embodiment, variant, derivatives and analogues of polypeptides/nucleotides will have about 70% identity with those sequences described herein. That is, 70% of the residues are the same. In a further embodiment, polypeptides/nucleotides will have greater than 75% identity. In a further embodiment, polypeptides/nucleotides will have greater than 80% identity. In a further embodiment, polypeptides/nucleotides will have greater than 85% identity. In a further embodiment, polypeptides/nucleotides will have greater than 90% identity. In a further embodiment, polypeptides/nucleotides will have greater than 95% identity. In a further embodiment, polypeptides/nucleotides will have greater than 99% identity.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G, eds., Humana Press, New Jersey (1994); Sequence Analysis in Molecular Biology, von Heinge, G, Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the disclosures of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12:387 (1984)), BLASTP, BLASTN and FASTA (Altschul, S. F. et al., J. Molec. Biol., 215:403 (1990)). The BLASTX program is publicly available from NCBI and other sources {BLAST Manual, Altschul, S. et al., NCVI NLM NIH Bethesda, Md. 20894, Altschul, S. F. et al., J. Molec. Biol., 215:403 (1990), the disclosures of which are incorporated herein by reference). These programs optimally align sequences using default gap weights in order to produce the highest level of sequence identity between the given and reference sequences. As an illustration, by a polynucleotide having a nucleotide sequence having at least, for example, 95% “sequence identity” to a reference nucleotide sequence, it is intended that the nucleotide sequence of the given polynucleotide is identical to the reference sequence except that the given polynucleotide sequence may include up to 5 point mutations per each 100 nucleotides of the reference nucleotide sequence. In other words, in a polynucleotide having a nucleotide sequence having at least 95% identity relative to the reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95% sequence identity to a reference amino acid sequence, it is intended that the given amino acid sequence of the polypeptide is identical to the reference sequence except that the given polypeptide sequence may include up to 5 amino acid alterations per each 100 amino acids of the reference amino acid sequence. In other words, to obtain a given polypeptide sequence having at least 95% sequence identity with a reference amino acid sequence, up to 5% of the amino acid residues in the reference sequence may be deleted or substituted with another amino acid, or a number of amino acids up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions that are not identical differ by conservative amino acid substitutions.

General methods regarding polynucleotides and polypeptides are described in: Sambrook et al., Molecular Cloning: A Laboratory Manual, 2nd ed., Cold Spring Harbor, N.Y., 1989; Current Protocols in Molecular Biology, edited by Ausubel F. M. et al., John Wiley and Sons, Inc. New York; PCR Cloning Protocols, from Molecular Cloning to Genetic Engineering, Edited by White B. A., Humana Press, Totowa, N.J., 1997, 490 pages; Protein Purification, Principles and Practices, Scopes R. K., Springer-Verlag, New York, 3rd Edition, 1993, 380 pages; Current Protocols in Immunology, edited by Coligan J. E. et al., John Wiley & Sons Inc., New York, which are herein incorporated by reference.

Methods involving gene targeting with parvovirus' including adeno-associate virus (AAV) are described in, for example, WO 98/48005 and WO 00/24917, which are incorporated herein by reference. Other methods involving gene targeting are disclosed in, for example, U.S. Pat. Nos. 6,528,313 and 6,528,314, which are incorporated herein by reference. Additional methods are described in Kohli et al., Nucl. Acids Res., 32:e3 (2004) and then modified by Topaloglu et al., Nucl Acids Res., 33:e158 (2005), Konishi et al., Nat. Protoc., 2:2865 (2007), Rago et al., Nat. Protoc., 2:2734 (2007), Zhang et al., Nat. Meth., 5:163 (2008) or Berdougo et al., Meth. Mol. Biol., 545:21 (2009), which are incorporated herein by reference.

The terms “comprises,” “comprising,” and the like can have the meaning ascribed to them in U.S. Patent Law and can mean “includes,” “including” and the like. As used herein, “including” or “includes” or the like means including, without limitation.

The Mechanism of rAAV-Mediated Gene Targeting in Human Somatic Cells

Somatic gene targeting in human cells has two general applications of importance and wide interest. One is the inactivation of genes (“knockouts”), a process utilized to delineate the loss-of-function phenotype(s) of a particular gene. The second application is the process of gene therapy (alternatively, “knock-ins”), which involves correcting a preexisting mutated allele(s) of a gene back to wild-type in order to ameliorate some pathological phenotype associated with the mutation. Both of these proceed through a form of DNA double-strand break repair known as homologous recombination (50). Although bacteria and lower eukaryotes utilize homologous recombination almost exclusively, a competing process, known as non-homologous end joining (26), predominates in higher eukaryotes and was presumed to prevent the use of gene targeting in human somatic cells in culture. A series of molecular and technical advances developed in the late 1980s (45, 47) and early 1990s (19, 61) disproved this notion, but still resulted in a process that was cumbersome, labor intensive, highly inefficient, and slow. Within the past decade, the use of new vectors such as rAAV (recombinant adeno-associated virus) (21) and new nucleases such as ZFNs (zinc finger nucleases) and TALENs (transcription activator-like effector nucleases) (59) have significantly brightened the outlook for this field (10) and resulted in gene modification systems that facilitate both gene knockouts and gene therapy modifications at robust levels. Thus, gene targeting in human somatic cells in culture has become not only feasible, but also relatively facile, and it harbingers a golden age for directed mutagenesis.

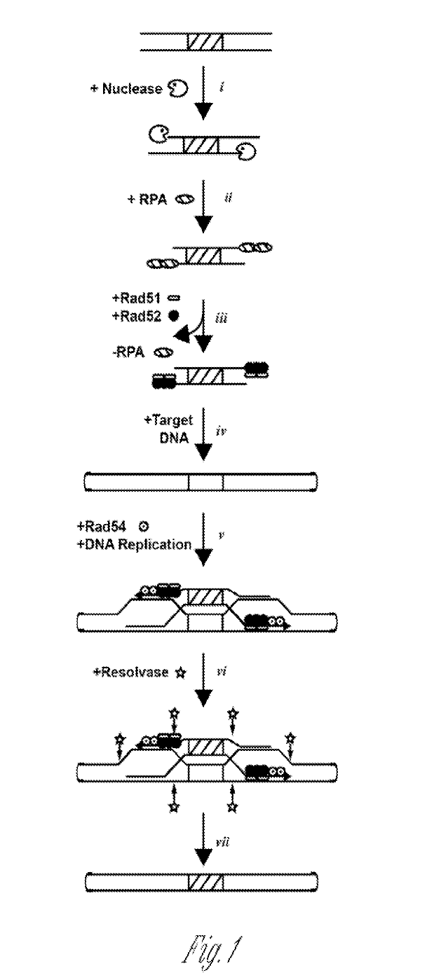

Although knockouts and knock-ins are, at the DNA level, reciprocal opposites of one another, they are mechanistically identical and utilize the same four basic steps: (i) a search for homologous sequences between the incoming donor DNA and the chromosomal DNA, (ii) breakage (usually in the form of DSBs (double-stranded breaks)) of the DNA at the site of targeting, (iii) exchange of DNA/genetic information between the donor DNA and the chromosomal DNA, and (iv) ligation of the broken chromosome to restore its structural integrity. Together, these four steps define a process referred to as HR (homologous recombination), which is needed for gene targeting to occur (50). Although the specifics of some of the steps in HR-facilitated gene targeting are still obscure, the basic process has been worked out, at least in yeast, in great detail (22, 51), and the mechanism seems generally applicable to mammals (25). In HR, the DNA ends of the in-coming DNA are likely resected to yield 3′-single-stranded DNA overhangs (FIG. 1i). Despite intense investigation, the identity of this nuclease(s) is still undetermined, although the MRN (Mre11/Rad50/Nbs1) complex, ExoI (exonuclease I) and CtIP (CtBP interacting protein) have been repeatedly implicated as the likely culprit(s) (50). The resulting overhangs are then coated by replication protein A (RPA; FIG. 1ii), a heterotrimeric single-stranded DNA-binding protein, which removes the secondary structures from the overhangs (16). RPA subsequently helps to recruit Rad51 (radiation-sensitive 51) and Rad52 to the overhangs, although it is itself displaced in the process (FIG. 1iii).

Rad51 is a strand-exchange protein in homologous recombination (20). It is used in the homology searches on the target DNA, i.e., the entire human genome (FIG. 1iv), that are needed to localize the incoming DNA to its specific, cognate chromosomal counterpart (49). In humans, there are at least seven Rad51 family members and almost all of them have been implicated in some aspect of HR and also in disease (52). Rad52 is an accessory factor for Rad51 and it facilitates strand exchange, probably by overcoming the inhibitory role of RPA (48). Strand invasion into the homologous chromosomal sequence involves Rad54 and DNA replication (FIG. 1v). Rad54 is a double-stranded DNA-dependent ATPase that can remodel chromatin, and it probably plays roles at several steps in the recombination process (13). In particular, Rad54 is used for stabilizing the Rad51-dependent joint molecule formation (FIG. 1v) as well as for promoting the disassembly of Rad51 following exchange (46). Gene targeting generates a complex structure (FIG. 1v) that is essentially identical to the linearized plasmid “ends-out” recombination intermediates that have been extensively defined in yeast (12). Ultimately, the resolution of this structure probably involves the participation of helicases of the RecQ (recombination defective Q) family (43) and the action of a resolvase(s) to repeatedly nick the strands (FIG. 1vi). In humans, there are at least 3 resolvase complexes that are overlapping in their activities (58). The resolution of the cross-stranded intermediates with crossovers generates a modified chromosome in which the original chromosomal sequences have been replaced by the sequences present on the incoming donor DNA (FIG. 1, vii). In summary, human somatic cells express all of the gene products needed to carry out classic dsDNA-mediated gene targeting.

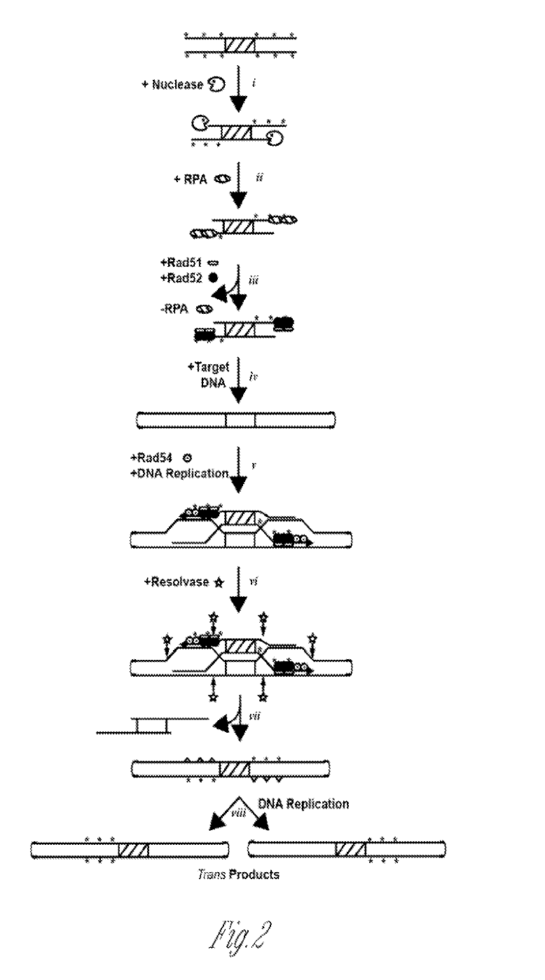

One of ways that was used to demonstrate that canonical gene targeting occurs through the two-ended, ends-out dsDNA mechanism outlined above (FIG. 1), was through the utilization of a donor dsDNA carrying SNPs (single nucleotide polymorphisms). When such a donor was used in yeast (22) or murine (1) cells, the resultant correctly gene targeted products carried the SNPs in a trans configuration, as the model would have predicted (FIG. 2). The mechanistic explanation for why trans products are observed results from two independent strand invasion events ((22); FIG. 2). As a result, the donor SNPs (*) that flank a drug selection marker (hatched box) are transferred only from one strand and generate an intermediate containing heteroduplex (inverted >) at the sites of the SNPs (FIG. 2vii). When this intermediate is resolved via DNA replication, separate products containing the SNPs in a trans configuration are generated (FIG. 2viii). Thus, the generation of trans recombination products from a SNP-marked donor vector is diagnostic for the canonical two-ended, ends-out dsDNA gene targeting mechanism (1, 12, 22).

In spite of the dogma that gene targeting in yeast and mammals proceeds essentially as described above (FIG. 2), ssDNA (single-stranded DNA) can also mediate, and/or be incorporated into, gene targeting products. Even in early work carried out in yeast, there were indications that ssDNA could facilitate (31) or end up in recombination products (24) and this led to the verification of an alternative form of HR, termed SSA (single-strand annealing) (8). These experiments were then extended to humans ((38) and reviewed in (17)). ssDNA incorporation has also been observed at high frequency in gene targeting experiments facilitated by ZFN-mediated DSBs (2). Despite these reports describing ssDNA utilization, the widely-held belief is that the two-ended, ends-out dsDNA mechanism is the predominate way in which gene targeting occurs in humans Assimilation of ssDNA is considered to be an interesting sidelight, but likely not relevant to major recombinational/gene targeting strategies.

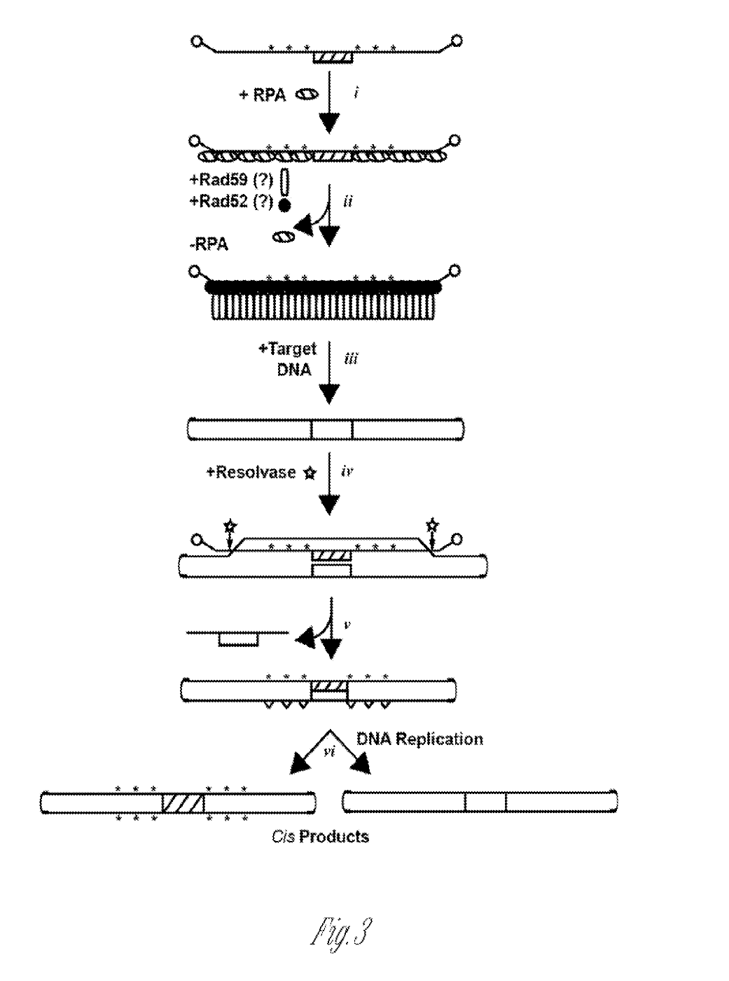

If ssDNA is used as a gene targeting intermediate, then cis, rather than trans (FIG. 2) products of recombination should be recovered (FIG. 3), as the incoming SNPs all reside on the same DNA strand as opposed to residing on separate strands as occurs in the two-ended, ends-out dsDNA mechanism (compare FIGS. 2 and 3). The mechanism by which ssDNA assimilation could occur is completely unknown. However, while not wanting to be bound by any theory, it is hypothesized herein that ssDNA can be coated by RPA (FIG. 3i). This DNA may then be a substrate either for Rad59, Rad52 or both in a process that could result in the loading of these proteins onto the DNA and the loss of RPA (FIG. 3ii). In mammals, Rad52 (Rad59 is a less well-studied Rad52 paralog) appears to be the major strand-annealing protein (33). Interaction of the Rad52/Rad59-coated ssDNA with a chromosome containing homologous DNA (FIG. 3iii) results in the formation of a D-loop structure. Resolution of this intermediate by resolvase (FIG. 3iv) may require two, as opposed to six (FIG. 2vi), cleavages. The recombinant product resulting from resolvase processing can contain significant heteroduplex (FIG. 3v). When this intermediate is resolved by DNA replication, two products would be generated (FIG. 3vi). One of these products, however, corresponds to an unaltered chromosome. The other product would contain a genetically-altered chromosome in which the SNPs flank the drug resistance marker in cis.

The above descriptions detail how a 2-ended, dsDNA model of gene targeting predicts trans products of recombination, whereas a ssDNA assimilation/annealing model predicts cis products of recombination. Layered on top of this, the MMR (mismatch repair) status of the cell being targeted can be relevant regardless of which model of gene targeting is occurring. MMR is a dedicated DNA repair process that removes the mismatched nucleotides that can (albeit rarely) become incorporated into nascent DNA during DNA replication (37). MMR does, however, also play a role in DNA recombination. Thus, homologous DNA strands (strands that are not identical) that engage in DNA recombination can generate transient dsDNA intermediates (called heteroduplexes) that contain DNA mismatches (FIG. 2, vii and FIG. 3v).

Herein it is demonstrated that rAAV, a single-stranded DNA virus that is used extensively in human gene targeting studies, targets DNA using a mechanism that resembles single-strand assimilation/annealing. This observation has important implications for improving not only rAAV-mediated gene targeting, but also for improving other forms of gene targeting where single-stranded DNA is utilized, or is an intermediate.

### Mismatch Repair

DNA mismatch repair is a system for recognizing and repairing the erroneous insertion, deletion and mis-incorporation of bases that can arise during DNA replication and recombination, as well as repairing some forms of DNA damage.

Mismatch repair is strand-specific. During DNA synthesis the newly synthesized (daughter) strand can include errors. In order to correct this, mismatch repair machinery distinguishes the newly synthesized strand from the template (parental). In gram-negative bacteria transient hemimethylation distinguishes the strands (the parental is methylated and daughter is not). In other prokaryotes and eukaryotes the exact mechanism for distinguishing parental from daughter strands is not clear.

There are a number of proteins involved in the mismatch repair process, including, but not limited to,

| MLH1 (mRNA NM_000249.3; protein NP_000240.1), | |
|---|---|
| (SEQ ID NO: 56) | |
| GAAGAGACCCAGCAACCCACAGAGTTGAGAAATTTGACTGGCATTCAAGCTGTCCAATCAATAGCTGCCGCTGAA | |
| GGGTGGGGCTGGATGGCGTAAGCTACAGCTGAAGGAAGAACGTGAGCACGAGGCACTGAGGTGATTGGCTGAAGG | |
| CACTTCCGTTGAGCATCTAGACGTTTCCTTGGCTCTTCTGGCGCCAAAATGTCGTTCGTGGCAGGGGTTATTCGG | |
| CGGCTGGACGAGACAGTGGTGAACCGCATCGCGGCGGGGGAAGTTATCCAGCGGCCAGCTAATGCTATCAAAGAG | |
| ATGATTGAGAACTGTTTAGATGCAAAATCCACAAGTATTCAAGTGATTGTTAAAGAGGGAGGCCTGAAGTTGATT | |
| CAGATCCAAACAATGGCACCGGGATCAGGAAAGAAGATCTGGATATTGTATGTGAAAGGTTCACTACTAGTAAAC | |
| TGCAGTCCTTTGAGGATTTAGCCAGTATTTCTACCTATGGCTTTCGAGGTGAGGCTTTGGCCAGCATAAGCCATG | |
| TGGCTCATGTTACTATTACAACGAAAACAGCTGATGGAAAGTGTGCATACAGAGCAAGTTACTCAGATGGAAAAC | |
| TGAAAGCCCCTCCTAAACCATGTGCTGGCAATCAAGGGACCCAGATCACGGTGGAGGACCTTTTTTACAACATAG | |
| CCACGAGGAGAAAAGCTTTAAAAAATCCAAGTGAAGAATATGGGAAAATTTTGGAAGTTGTTGGCAGGTATTCAG | |
| TACACAATGCAGGCATTAGTTTCTCAGTTAAAAAACAAGGAGAGACAGTAGCTGATGTTAGGACACTACCCAATG | |
| CCTCAACCGTGGACAATATTCGCTCCATCTTTGGAAATGCTGTTAGTCGAGAACTGATAGAAATTGGATGTGAGG | |
| ATAAAACCCTAGCCTTCAAAATGAATGGTTACATATCCAATGCAAACTACTCAGTGAAGAAGTGCATCTTCTTAC | |
| TCTTCATCAACCATCGTCTGGTAGAATCAACTTCCTTGAGAAAAGCCATAGAAACAGTGTATGC | |
| AGCCTATTTGCCCAAAAACACACACCCATTCCTGTACCTCAGTTTAGAAATCAGTCCCCAGAATGTGGATGTTAA | |
| TGTGCACCCCACAAAGCATGAAGTTCACTTCCTGCACGAGGAGAGCATCCTGGAGCGGGTGCAGCAGCACATCGA | |
| GAGCAAGCTCCTGGGCTCCAATTCCTCCAGGATGTACTTCACCCAGACTTTGCTACCAGGACTTGCTGGCCCCTC | |
| TGGGGAGATGGTTAAATCCACAACAAGTCTGACCTCGTCTTCTACTTCTGGAAGTAGTGATAAGGTCTATGCCCA | |
| CCAGATGGTTCGTACAGATTCCCGGGAACAGAAGCTTGATGCATTTCTGCAGCCTCTGAGCAAACCCCTGTCCAG | |
| TCAGCCCCAGGCCATTGTCACAGAGGATAAGACAGATATTTCTAGTGGCAGGGCTAGGCAGCAAGATGAGGAGAT | |
| GCTTGAACTCCCAGCCCCTGCTGAAGTGGCTGCCAAAAATCAGAGCTTGGAGGGGGATACAACAAAGGGGACTTC | |
| AGAAATGTCAGAGAAGAGAGGACCTACTTCCAGCAACCCCAGAAAGAGACATCGGGAAGATTCTGATGTGGAAAT | |
| GGTGGAAGATGATTCCCGAAAGGAAATGACTGCAGCTTGTACCCCCCGGAGAAGGATCATTAACCTCACTAGTGT | |
| TTTGAGTCTCCAGGAAGAAATTAATGAGCAGGGACATGAGGTTCTCCGGGAGATGTTGCATAACCACTCCTTCGT | |
| GGGCTGTGTGAATCCTCAGTGGGCCTTGGCACAGCATCAAACCAAGTTATACCTTCTCAACACCACCAAGCTTAG | |
| TGAAGAACTGTTCTACCAGATACTCATTTATGATTTTGCCAATTTTGGTGTTCTCAGGTTATCGGAGCCAGCACC | |
| GCTCTTTGACCTTGCCATGCTTGCCTTAGATAGTCCAGAGAGTGGCTGGACAGAGGAAGATGGTCCCAAAGAAGG | |
| ACTTGCTGAATACATTGTTGAGTTTCTGAAGAAGAAGGCTGAGATGCTTGCAGACTATTTCTCTT | |
| TGGAAATTGATGAGGAAGGGAACCTGATTGGATTACCCCTTCTGATTGACAACTATGTGCCCCCTTTGGAGGGAC | |
| TGCCTATCTTCATTCTTCGACTAGCCACTGAGGTGAATTGGGACGAAGAAAAGGAATGTTTTGAAAGCCTCAGTA | |
| AAGAATGCGCTATGTTCTATTCCATCCGGAAGCAGTACATATCTGAGGAGTCGACCCTCTCAGGCCAGCAGAGTG | |
| AAGTGCCTGGCTCCATTCCAAACTCCTGGAAGTGGACTGTGGAACACATTGTCTATAAAGCCTTGCGCTCACACA | |
| TTCTGCCTCCTAAACATTTCACAGAAGATGGAAATATCCTGCAGCTTGCTAACCTGCCTGATCTATACAAAGTCT | |
| TTGAGAGGTGTTAAATATGGTTATTTATGCACTGTGGGATGTGTTCTTCTTTCTCTGTATTCCGATACAAAGTGT | |
| TGTATCAAAGTGTGATATACAAAGTGTACCAACATAAGTGTTGGTAGCACTTAAGACTTATACTTGCCTTCTGAT | |
| AGTATTCCTTTATACACAGTGGATTGATTATAAATAAATAGATGTGTCTTAACATAA. | |
| PMS2 ((mRNA NM_000535.5; protein NP_000526.1) this gene is one | |
| of the PMS2 gene family members which are found in clusters on | |
| chromosome 7; the product of this gene is involved in DNA mis- | |
| match repair and the protein forms a heterodimer with MLH1 and | |
| this complex interacts with MSH2 bound to mismatched bases), | |
| (SEQ ID NO: 57) | |
| 1 | agccaatggg agttcaggag gcggagcgcc tgtgggagcc ctggagggaa ctttcccagt |
| 61 | ccccgaggcg gatcgggtgt tgcatccatg gagcgagctg agagctcgag tacagaacct |
| 121 | gctaaggcca tcaaacctat tgatcggaag tcagtccatc agatttgctc tgggcaggtg |
| 181 | gtactgagtc taagcactgc ggtaaaggag ttagtagaaa acagtctgga tgctggtgcc |
| 241 | actaatattg atctaaagct taaggactat ggagtggatc ttattgaagt ttcagacaat |
| 301 | ggatgtgggg tagaagaaga aaacttcgaa ggcttaactc tgaaacatca cacatctaag |
| 361 | attcaagagt ttgccgacct aactcaggtt gaaacttttg gctttcgggg ggaagctctg |
| 421 | agctcacttt gtgcactgag cgatgtcacc atttctacct gccacgcatc ggcgaaggtt |
| 481 | ggaactcgac tgatgtttga tcacaatggg aaaattatcc agaaaacccc ctacccccgc |
| 541 | cccagaggga ccacagtcag cgtgcagcag ttattttcca cactacctgt gcgccataag |
| 601 | gaatttcaaa ggaatattaa gaaggagtat gccaaaatgg tccaggtctt acatgcatac |
| 661 | tgtatcattt cagcaggcat ccgtgtaagt tgcaccaatc agcttggaca aggaaaacga |
| 721 | cagcctgtgg tatgcacagg tggaagcccc agcataaagg aaaatatcgg ctctgtgttt |
| 781 | gggcagaagc agttgcaaag cctcattcct tttgttcagc tgccccctag tgactccgtg |
| 841 | tgtgaagagt acggtttgag ctgttccgat gctctgcata atctttttta catctcaggt |
| 901 | ttcatttcac aatgcacgca tggagttgga aggagttcaa cagacagaca gtttttcttt |
| 961 | atcaaccggc ggccttgtga cccagcaaag gtctgcagac tcgtgaatga ggtctaccac |
| 1021 | atgtataatc gacaccagta tccatttgtt gttcttaaca tttctgttga ttcagaatgc |
| 1081 | gttgatatca atgttactcc agataaaagg caaattttgc tacaagagga aaagcttttg |
| 1141 | ttggcagttt taaagacctc tttgatagga atgtttgata gtgatgtcaa caagctaaat |
| 1201 | gtcagtcagc agccactgct ggatgttgaa ggtaacttaa taaaaatgca tgcagcggat |
| 1261 | ttggaaaagc ccatggtaga aaagcaggat caatcccctt cattaaggac tggagaagaa |
| 1321 | aaaaaagacg tgtccatttc cagactgcga gaggcctttt ctcttcgtca cacaacagag |
| 1381 | aacaagcctc acagcccaaa gactccagaa ccaagaagga gccctctagg acagaaaagg |
| 1441 | ggtatgctgt cttctagcac ttcaggtgcc atctctgaca aaggcgtcct gagacctcag |
| 1501 | aaagaggcag tgagttccag tcacggaccc agtgacccta cggacagagc ggaggtggag |
| 1561 | aaggactcgg ggcacggcag cacttccgtg gattctgagg ggttcagcat cccagacacg |
| 1621 | ggcagtcact gcagcagcga gtatgcggcc agctccccag gggacagggg ctcgcaggaa |
| 1681 | catgtggact ctcaggagaa agcgcctgaa actgacgact ctttttcaga tgtggactgc |
| 1741 | cattcaaacc aggaagatac cggatgtaaa tttcgagttt tgcctcagcc aactaatctc |
| 1801 | gcaaccccaa acacaaagcg ttttaaaaaa gaagaaattc tttccagttc tgacatttgt |
| 1861 | caaaagttag taaatactca ggacatgtca gcctctcagg ttgatgtagc tgtgaaaatt |
| 1921 | aataagaaag ttgtgcccct ggacttttct atgagttctt tagctaaacg aataaagcag |
| 1981 | ttacatcatg aagcacagca aagtgaaggg gaacagaatt acaggaagtt tagggcaaag |
| 2041 | atttgtcctg gagaaaatca agcagccgaa gatgaactaa gaaaagagat aagtaaaacg |
| 2101 | atgtttgcag aaatggaaat cattggtcag tttaacctgg gatttataat aaccaaactg |
| 2161 | aatgaggata tcttcatagt ggaccagcat gccacggacg agaagtataa cttcgagatg |
| 2221 | ctgcagcagc acaccgtgct ccaggggcag aggctcatag cacctcagac tctcaactta |
| 2281 | actgctgtta atgaagctgt tctgatagaa aatctggaaa tatttagaaa gaatggcttt |
| 2341 | gattttgtta tcgatgaaaa tgctccagtc actgaaaggg ctaaactgat ttccttgcca |
| 2401 | actagtaaaa actggacctt cggaccccag gacgtcgatg aactgatctt catgctgagc |
| 2461 | gacagccctg gggtcatgtg ccggccttcc cgagtcaagc agatgtttgc ctccagagcc |
| 2521 | tgccggaagt cggtgatgat tgggactgct cttaacacaa gcgagatgaa gaaactgatc |
| 2581 | acccacatgg gggagatgga ccacccctgg aactgtcccc atggaaggcc aaccatgaga |
| 2641 | cacatcgcca acctgggtgt catttctcag aactgaccgt agtcactgta tggaataatt |
| 2701 | ggttttatcg cagattttta tgttttgaaa gacagagtct tcactaacct tttttgtttt |
| 2761 | aaaatgaacc tgctacttaa aaaaaataca catcacaccc atttaaaagt gatcttgaga |
| 2821 | accttttcaa accagaaaaa aaaaaaaaaa a |
| MSH2 (mRNA NM_000251.1; NP_000242.1), | |
| (SEQ ID NO: 58) | |
| 1 | ggcgggaaac agcttagtgg gtgtggggtc gcgcattttc ttcaaccagg aggtgaggag |
| 61 | gtttcgacat ggcggtgcag ccgaaggaga cgctgcagtt ggagagcgcg gccgaggtcg |
| 121 | gcttcgtgcg cttctttcag ggcatgccgg agaagccgac caccacagtg cgccttttcg |
| 181 | accggggcga cttctatacg gcgcacggcg aggacgcgct gctggccgcc cgggaggtgt |
| 241 | tcaagaccca gggggtgatc aagtacatgg ggccggcagg agcaaagaat ctgcagagtg |
| 301 | ttgtgcttag taaaatgaat tttgaatctt ttgtaaaaga tcttcttctg gttcgtcagt |
| 361 | atagagttga agtttataag aatagagctg gaaataaggc atccaaggag aatgattggt |
| 421 | atttggcata taaggcttct cctggcaatc tctctcagtt tgaagacatt ctctttggta |
| 481 | acaatgatat gtcagcttcc attggtgttg tgggtgttaa aatgtccgca gttgatggcc |
| 541 | agagacaggt tggagttggg tatgtggatt ccatacagag gaaactagga ctgtgtgaat |
| 601 | tccctgataa tgatcagttc tccaatcttg aggctctcct catccagatt ggaccaaagg |
| 661 | aatgtgtttt acccggagga gagactgctg gagacatggg gaaactgaga cagataattc |
| 721 | aaagaggagg aattctgatc acagaaagaa aaaaagctga cttttccaca aaagacattt |
| 781 | atcaggacct caaccggttg ttgaaaggca aaaagggaga gcagatgaat agtgctgtat |
| 841 | tgccagaaat ggagaatcag gttgcagttt catcactgtc tgcggtaatc aagtttttag |
| 901 | aactcttatc agatgattcc aactttggac agtttgaact gactactttt gacttcagcc |
| 961 | agtatatgaa attggatatt gcagcagtca gagcccttaa cctttttcag ggttctgttg |
| 1021 | aagataccac tggctctcag tctctggctg ccttgctgaa taagtgtaaa acccctcaag |
| 1081 | gacaaagact tgttaaccag tggattaagc agcctctcat ggataagaac agaatagagg |
| 1141 | agagattgaa tttagtggaa gcttttgtag aagatgcaga attgaggcag actttacaag |
| 1201 | aagatttact tcgtcgattc ccagatctta accgacttgc caagaagttt caaagacaag |
| 1261 | cagcaaactt acaagattgt taccgactct atcagggtat aaatcaacta cctaatgtta |
| 1321 | tacaggctct ggaaaaacat gaaggaaaac accagaaatt attgttggca gtttttgtga |
| 1381 | ctcctcttac tgatcttcgt tctgacttct ccaagtttca ggaaatgata gaaacaactt |
| 1441 | tagatatgga tcaggtggaa aaccatgaat tccttgtaaa accttcattt gatcctaatc |
| 1501 | tcagtgaatt aagagaaata atgaatgact tggaaaagaa gatgcagtca acattaataa |
| 1561 | gtgcagccag agatcttggc ttggaccctg gcaaacagat taaactggat tccagtgcac |
| 1621 | agtttggata ttactttcgt gtaacctgta aggaagaaaa agtccttcgt aacaataaaa |
| 1681 | actttagtac tgtagatatc cagaagaatg gtgttaaatt taccaacagc aaattgactt |
| 1741 | ctttaaatga agagtatacc aaaaataaaa cagaatatga agaagcccag gatgccattg |
| 1801 | ttaaagaaat tgtcaatatt tcttcaggct atgtagaacc aatgcagaca ctcaatgatg |
| 1861 | tgttagctca gctagatgct gttgtcagct ttgctcacgt gtcaaatgga gcacctgttc |
| 1921 | catatgtacg accagccatt ttggagaaag gacaaggaag aattatatta aaagcatcca |
| 1981 | ggcatgcttg tgttgaagtt caagatgaaa ttgcatttat tcctaatgac gtatactttg |
| 2041 | aaaaagataa acagatgttc cacatcatta ctggccccaa tatgggaggt aaatcaacat |
| 2101 | atattcgaca aactggggtg atagtactca tggcccaaat tgggtgtttt gtgccatgtg |
| 2161 | agtcagcaga agtgtccatt gtggactgca tcttagcccg agtaggggct ggtgacagtc |
| 2221 | aattgaaagg agtctccacg ttcatggctg aaatgttgga aactgcttct atcctcaggt |
| 2281 | ctgcaaccaa agattcatta ataatcatag atgaattggg aagaggaact tctacctacg |
| 2341 | atggatttgg gttagcatgg gctatatcag aatacattgc aacaaagatt ggtgcttttt |
| 2401 | gcatgtttgc aacccatttt catgaactta ctgccttggc caatcagata ccaactgtta |
| 2461 | ataatctaca tgtcacagca ctcaccactg aagagacctt aactatgctt tatcaggtga |
| 2521 | agaaaggtgt ctgtgatcaa agttttggga ttcatgttgc agagcttgct aatttcccta |
| 2581 | agcatgtaat agagtgtgct aaacagaaag ccctggaact tgaggagttt cagtatattg |
| 2641 | gagaatcgca aggatatgat atcatggaac cagcagcaaa gaagtgctat ctggaaagag |
| 2701 | agcaaggtga aaaaattatt caggagttcc tgtccaaggt gaaacaaatg ccctttactg |
| 2761 | aaatgtcaga agaaaacatc acaataaagt taaaacagct aaaagctgaa gtaatagcaa |
| 2821 | agaataatag ctttgtaaat gaaatcattt cacgaataaa agttactacg tgaaaaatcc |
| 2881 | cagtaatgga atgaaggtaa tattgataag ctattgtctg taatagtttt atattgtttt |
| 2941 | atattaaccc tttttccata gtgttaactg tcagtgccca tgggctatca acttaataag |
| 3001 | atatttagta atattttact ttgaggacat tttcaaagat ttttattttg aaaaatgaga |
| 3061 | gctgtaactg aggactgttt gcaattgaca taggcaataa taagtgatgt gctgaatttt |
| 3121 | ataaataaaa tcatgtagtt tgtgg |
| MSH6 (mRNA NM_000179.2; protein NP_000170.1), | |
| (SEQ ID NO: 59) | |
| 1 | ggcgaggcgc ctgttgattg gccactgggg cccgggttcc tccggcggag cgcgcctccc |
| 61 | cccagatttc ccgccagcag gagccgcgcg gtagatgcgg tgcttttagg agctccgtcc |
| 121 | gacagaacgg ttgggccttg ccggctgtcg gtatgtcgcg acagagcacc ctgtacagct |
| 181 | tcttccccaa gtctccggcg ctgagtgatg ccaacaaggc ctcggccagg gcctcacgcg |
| 241 | aaggcggccg tgccgccgct gcccccgggg cctctccttc cccaggcggg gatgcggcct |
| 301 | ggagcgaggc tgggcctggg cccaggccct tggcgcgctc cgcgtcaccg cccaaggcga |
| 361 | agaacctcaa cggagggctg cggagatcgg tagcgcctgc tgcccccacc agttgtgact |
| 421 | tctcaccagg agatttggtt tgggccaaga tggagggtta cccctggtgg ccttgtctgg |
| 481 | tttacaacca cccctttgat ggaacattca tccgcgagaa agggaaatca gtccgtgttc |
| 541 | atgtacagtt ttttgatgac agcccaacaa ggggctgggt tagcaaaagg cttttaaagc |
| 601 | catatacagg ttcaaaatca aaggaagccc agaagggagg tcatttttac agtgcaaagc |
| 661 | ctgaaatact gagagcaatg caacgtgcag atgaagcctt aaataaagac aagattaaga |
| 721 | ggcttgaatt ggcagtttgt gatgagccct cagagccaga agaggaagaa gagatggagg |
| 781 | taggcacaac ttacgtaaca gataagagtg aagaagataa tgaaattgag agtgaagagg |
| 841 | aagtacagcc taagacacaa ggatctaggc gaagtagccg ccaaataaaa aaacgaaggg |
| 901 | tcatatcaga ttctgagagt gacattggtg gctctgatgt ggaatttaag ccagacacta |
| 961 | aggaggaagg aagcagtgat gaaataagca gtggagtggg ggatagtgag agtgaaggcc |
| 1021 | tgaacagccc tgtcaaagtt gctcgaaagc ggaagagaat ggtgactgga aatggctctc |
| 1081 | ttaaaaggaa aagctctagg aaggaaacgc cctcagccac caaacaagca actagcattt |
| 1141 | catcagaaac caagaatact ttgagagctt tctctgcccc tcaaaattct gaatcccaag |
| 1201 | cccacgttag tggaggtggt gatgacagta gtcgccctac tgtttggtat catgaaactt |
| 1261 | tagaatggct taaggaggaa aagagaagag atgagcacag gaggaggcct gatcaccccg |
| 1321 | attttgatgc atctacactc tatgtgcctg aggatttcct caattcttgt actcctggga |
| 1381 | tgaggaagtg gtggcagatt aagtctcaga actttgatct tgtcatctgt tacaaggtgg |
| 1441 | ggaaatttta tgagctgtac cacatggatg ctcttattgg agtcagtgaa ctggggctgg |
| 1501 | tattcatgaa aggcaactgg gcccattctg gctttcctga aattgcattt ggccgttatt |
| 1561 | cagattccct ggtgcagaag ggctataaag tagcacgagt ggaacagact gagactccag |
| 1621 | aaatgatgga ggcacgatgt agaaagatgg cacatatatc caagtatgat agagtggtga |
| 1681 | ggagggagat ctgtaggatc attaccaagg gtacacagac ttacagtgtg ctggaaggtg |
| 1741 | atccctctga gaactacagt aagtatcttc ttagcctcaa agaaaaagag gaagattctt |
| 1801 | ctggccatac tcgtgcatat ggtgtgtgct ttgttgatac ttcactggga aagtttttca |
| 1861 | taggtcagtt ttcagatgat cgccattgtt cgagatttag gactctagtg gcacactatc |
| 1921 | ccccagtaca agttttattt gaaaaaggaa atctctcaaa ggaaactaaa acaattctaa |
| 1981 | agagttcatt gtcctgttct cttcaggaag gtctgatacc cggctcccag ttttgggatg |
| 2041 | catccaaaac tttgagaact ctccttgagg aagaatattt tagggaaaag ctaagtgatg |
| 2101 | gcattggggt gatgttaccc caggtgctta aaggtatgac ttcagagtct gattccattg |
| 2161 | ggttgacacc aggagagaaa agtgaattgg ccctctctgc tctaggtggt tgtgtcttct |
| 2221 | acctcaaaaa atgccttatt gatcaggagc ttttatcaat ggctaatttt gaagaatata |
| 2281 | ttcccttgga ttctgacaca gtcagcacta caagatctgg tgctatcttc accaaagcct |
| 2341 | atcaacgaat ggtgctagat gcagtgacat taaacaactt ggagattttt ctgaatggaa |
| 2401 | caaatggttc tactgaagga accctactag agagggttga tacttgccat actccttttg |
| 2461 | gtaagcggct cctaaagcaa tggctttgtg ccccactctg taaccattat gctattaatg |
| 2521 | atcgtctaga tgccatagaa gacctcatgg ttgtgcctga caaaatctcc gaagttgtag |
| 2581 | agcttctaaa gaagcttcca gatcttgaga ggctactcag taaaattcat aatgttgggt |
| 2641 | ctcccctgaa gagtcagaac cacccagaca gcagggctat aatgtatgaa gaaactacat |
| 2701 | acagcaagaa gaagattatt gattttcttt ctgctctgga aggattcaaa gtaatgtgta |
| 2761 | aaattatagg gatcatggaa gaagttgctg atggttttaa gtctaaaatc cttaagcagg |
| 2821 | tcatctctct gcagacaaaa aatcctgaag gtcgttttcc tgatttgact gtagaattga |
| 2881 | accgatggga tacagccttt gaccatgaaa aggctcgaaa gactggactt attactccca |
| 2941 | aagcaggctt tgactctgat tatgaccaag ctcttgctga cataagagaa aatgaacaga |
| 3001 | gcctcctgga atacctagag aaacagcgca acagaattgg ctgtaggacc atagtctatt |
| 3061 | gggggattgg taggaaccgt taccagctgg aaattcctga gaatttcacc actcgcaatt |
| 3121 | tgccagaaga atacgagttg aaatctacca agaagggctg taaacgatac tggaccaaaa |
| 3181 | ctattgaaaa gaagttggct aatctcataa atgctgaaga acggagggat gtatcattga |
| 3241 | aggactgcat gcggcgactg ttctataact ttgataaaaa ttacaaggac tggcagtctg |
| 3301 | ctgtagagtg tatcgcagtg ttggatgttt tactgtgcct ggctaactat agtcgagggg |
| 3361 | gtgatggtcc tatgtgtcgc ccagtaattc tgttgccgga agataccccc cccttcttag |
| 3421 | agcttaaagg atcacgccat ccttgcatta cgaagacttt ttttggagat gattttattc |
| 3481 | ctaatgacat tctaataggc tgtgaggaag aggagcagga aaatggcaaa gcctattgtg |
| 3541 | tgcttgttac tggaccaaat atggggggca agtctacgct tatgagacag gctggcttat |
| 3601 | tagctgtaat ggcccagatg ggttgttacg tccctgctga agtgtgcagg ctcacaccaa |
| 3661 | ttgatagagt gtttactaga cttggtgcct cagacagaat aatgtcaggt gaaagtacat |
| 3721 | tttttgttga attaagtgaa actgccagca tactcatgca tgcaacagca cattctctgg |
| 3781 | tgcttgtgga tgaattagga agaggtactg caacatttga tgggacggca atagcaaatg |
| 3841 | cagttgttaa agaacttgct gagactataa aatgtcgtac attattttca actcactacc |
| 3901 | attcattagt agaagattat tctcaaaatg ttgctgtgcg cctaggacat atggcatgca |
| 3961 | tggtagaaaa tgaatgtgaa gaccccagcc aggagactat tacgttcctc tataaattca |
| 4021 | ttaagggagc ttgtcctaaa agctatggct ttaatgcagc aaggcttgct aatctcccag |
| 4081 | aggaagttat tcaaaaggga catagaaaag caagagaatt tgagaagatg aatcagtcac |
| 4141 | tacgattatt tcgggaagtt tgcctggcta gtgaaaggtc aactgtagat gctgaagctg |
| 4201 | tccataaatt gctgactttg attaaggaat tatagactga ctacattgga agctttgagt |
| 4261 | tgacttctga caaaggtggt aaattcagac aacattatga tctaataaac tttatttttt |
| 4321 | aaaaatgaaa aaaaaaaaaa aaaaaaaaaa aaaaaaaaaa aaaaaaaaaa aaaaaaaaaa |
| 4381 | aaaaaaaaaa aaaaaaaaaa aaaaaaaaaa aaaaaaaaaa aaaaaaaaaa aaaaa |
| MSH3 (mRNA NM_002439; protein NP_002430), | |
| (SEQ ID NO: 60) | |
| 1 | ccgcagacgc ctgggaactg cggccgcggg ctcgcgctcc tcgccaggcc ctgccgccgg |
| 61 | gctgccatcc ttgccctgcc atgtctcgcc ggaagcctgc gtcgggcggc ctcgctgcct |
| 121 | ccagctcagc ccctgcgagg caagcggttt tgagccgatt cttccagtct acgggaagcc |
| 181 | tgaaatccac ctcctcctcc acaggtgcag ccgaccaggt ggaccctggc gctgcagcgg |
| 241 | ctgcagcggc cgcagcggcc gcagcgcccc cagcgccccc agctcccgcc ttcccgcccc |
| 301 | agctgccgcc gcacatagct acagaaattg acagaagaaa gaagagacca ttggaaaatg |
| 361 | atgggcctgt taaaaagaaa gtaaagaaag tccaacaaaa ggaaggagga agtgatctgg |
| 421 | gaatgtctgg caactctgag ccaaagaaat gtctgaggac caggaatgtt tcaaagtctc |
| 481 | tggaaaaatt gaaagaattc tgctgcgatt ctgcccttcc tcaaagtaga gtccagacag |
| 541 | aatctctgca ggagagattt gcagttctgc caaaatgtac tgattttgat gatatcagtc |
| 601 | ttctacacgc aaagaatgca gtttcttctg aagattcgaa acgtcaaatt aatcaaaagg |
| 661 | acacaacact ttttgatctc agtcagtttg gatcatcaaa tacaagtcat gaaaatttac |
| 721 | agaaaactgc ttccaaatca gctaacaaac ggtccaaaag catctatacg ccgctagaat |
| 781 | tacaatacat agaaatgaag cagcagcaca aagatgcagt tttgtgtgtg gaatgtggat |
| 841 | ataagtatag attctttggg gaagatgcag agattgcagc ccgagagctc aatatttatt |
| 901 | gccatttaga tcacaacttt atgacagcaa gtatacctac tcacagactg tttgttcatg |
| 961 | tacgccgcct ggtggcaaaa ggatataagg tgggagttgt gaagcaaact gaaactgcag |
| 1021 | cattaaaggc cattggagac aacagaagtt cactcttttc ccggaaattg actgcccttt |
| 1081 | atacaaaatc tacacttatt ggagaagatg tgaatcccct aatcaagctg gatgatgctg |
| 1141 | taaatgttga tgagataatg actgatactt ctaccagcta tcttctgtgc atctctgaaa |
| 1201 | ataaggaaaa tgttagggac aaaaaaaagg gcaacatttt tattggcatt gtgggagtgc |
| 1261 | agcctgccac aggcgaggtt gtgtttgata gtttccagga ctctgcttct cgttcagagc |
| 1321 | tagaaacccg gatgtcaagc ctgcagccag tagagctgct gcttccttcg gccttgtccg |
| 1381 | agcaaacaga ggcgctcatc cacagagcca catctgttag tgtgcaggat gacagaattc |
| 1441 | gagtcgaaag gatggataac atttattttg aatacagcca tgctttccag gcagttacag |
| 1501 | agttttatgc aaaagataca gttgacatca aaggttctca aattatttct ggcattgtta |
| 1561 | acttagagaa gcctgtgatt tgctctttgg ctgccatcat aaaatacctc aaagaattca |
| 1621 | acttggaaaa gatgctctcc aaacctgaga attttaaaca gctatcaagt aaaatggaat |
| 1681 | ttatgacaat taatggaaca acattaagga atctggaaat cctacagaat cagactgata |
| 1741 | tgaaaaccaa aggaagtttg ctgtgggttt tagaccacac taaaacttca tttgggagac |
| 1801 | ggaagttaaa gaagtgggtg acccagccac tccttaaatt aagggaaata aatgcccggc |
| 1861 | ttgatgctgt atcggaagtt ctccattcag aatctagtgt gtttggtcag atagaaaatc |
| 1921 | atctacgtaa attgcccgac atagagaggg gactctgtag catttatcac aaaaaatgtt |
| 1981 | ctacccaaga gttcttcttg attgtcaaaa ctttatatca cctaaagtca gaatttcaag |
| 2041 | caataatacc tgctgttaat tcccacattc agtcagactt gctccggacc gttattttag |
| 2101 | aaattcctga actcctcagt ccagtggagc attacttaaa gatactcaat gaacaagctg |
| 2161 | ccaaagttgg ggataaaact gaattattta aagacctttc tgacttccct ttaataaaaa |
| 2221 | agaggaagga tgaaattcaa ggtgttattg acgagatccg aatgcatttg caagaaatac |
| 2281 | gaaaaatact aaaaaatcct tctgcacaat atgtgacagt atcaggacag gagtttatga |
| 2341 | tagaaataaa gaactctgct gtatcttgta taccaactga ttgggtaaag gttggaagca |
| 2401 | caaaagctgt gagccgcttt cactctcctt ttattgtaga aaattacaga catctgaatc |
| 2461 | agctccggga gcagctagtc cttgactgca gtgctgaatg gcttgatttt ctagagaaat |
| 2521 | tcagtgaaca ttatcactcc ttgtgtaaag cagtgcatca cctagcaact gttgactgca |
| 2581 | ttttctccct ggccaaggtc gctaagcaag gagattactg cagaccaact gtacaagaag |
| 2641 | aaagaaaaat tgtaataaaa aatggaaggc accctgtgat tgatgtgttg ctgggagaac |
| 2701 | aggatcaata tgtcccaaat aatacagatt tatcagagga ctcagagaga gtaatgataa |
| 2761 | ttaccggacc aaacatgggt ggaaagagct cctacataaa acaagttgca ttgattacca |
| 2821 | tcatggctca gattggctcc tatgttcctg cagaagaagc gacaattggg attgtggatg |
| 2881 | gcattttcac aaggatgggt gctgcagaca atatatataa aggacagagt acatttatgg |
| 2941 | aagaactgac tgacacagca gaaataatca gaaaagcaac atcacagtcc ttggttatct |
| 3001 | tggatgaact aggaagaggg acgagcactc atgatggaat tgccattgcc tatgctacac |
| 3061 | ttgagtattt catcagagat gtgaaatcct taaccctgtt tgtcacccat tatccgccag |
| 3121 | tttgtgaact agaaaaaaat tactcacacc aggtggggaa ttaccacatg ggattcttgg |
| 3181 | tcagtgagga tgaaagcaaa ctggatccag gcgcagcaga acaagtccct gattttgtca |
| 3241 | ccttccttta ccaaataact agaggaattg cagcaaggag ttatggatta aatgtggcta |
| 3301 | aactagcaga tgttcctgga gaaattttga agaaagcagc tcacaagtca aaagagctgg |
| 3361 | aaggattaat aaatacgaaa agaaagagac tcaagtattt tgcaaagtta tggacgatgc |
| 3421 | ataatgcaca agacctgcag aagtggacag aggagttcaa catggaagaa acacagactt |
| 3481 | ctcttcttca ttaaaatgaa gactacattt gtgaacaaaa aatggagaat taaaaatacc |
| 3541 | aactgtacaa aataactctc cagtaacagc ctatctttgt gtgacatgtg agcataaaat |
| 3601 | tatgaccatg gtatattcct attggaaaca gagaggtttt tctgaagaca gtctttttca |
| 3661 | agtttctgtc ttcctaactt ttctacgtat aaacactctt gaatagactt ccactttgta |
| 3721 | attagaaaat tttatggaca gtaagtccag taaagcctta agtggcagaa tataattccc |
| 3781 | aagcttttgg agggtgatat aaaaatttac ttgatatttt tatttgtttc agttcagata |
| 3841 | attggcaact gggtgaatct ggcaggaatc tatccattga actaaaataa ttttattatg |
| 3901 | caaccagttt atccaccaag aacataagaa ttttttataa gtagaaagaa ttggccaggc |
| 3961 | atggtggctc atgcctgtaa tcccagcact ttgggaggcc aaggtaggca gatcacctga |
| 4021 | ggtcaggagt tcaagaccag cctggccaac atggcaaaac cccatcttta ctaaaaatat |
| 4081 | aaagtacatc tctactaaaa atacgaaaaa attagctggg catggtggcg cacacctgta |
| 4141 | gtcccagcta ctccggaggc tgaggcagga gaatctcttg aacctgggag gcggaggttg |
| 4201 | caatgagccg agatcacgtc actgcactcc agcttgggca acagagcaag actccatctc |
| 4261 | aaaaaaaaaa aaagaaaaaa gaaaagaaat agaattatca agcttttaaa aactagagca |
| 4321 | cagaaggaat aaggtcatga aatttaaaag gttaaatatt gtcataggat taagcagttt |
| 4381 | aaagattgtt ggatgaaatt atttgtcatt cattcaagta ataaatattt aatgaatact |
| 4441 | tgctataaaa aaaaaaaaaa aaaaaaaaaa aa |
| PMS1 (mRNA NM_000534.4; protein NP_000525.1), | |
| (SEQ ID NO: 61) | |
| 1 | ggcaagacaa cgaggatttg cgtagggggc gagcctctga ggccacttgg ctcttacggc |
| 61 | cacgcagggc gccgcagatg cagccggagc ccgcttttcc ctctcaggac gacccctagg |
| 121 | ccgccagcag ttccctaccg acgaaggcga ctgtacagcg tccaccgcgt tcgtgcccac |
| 181 | ttacccgccg ccccactccg ggccgccggc tcgcagcagg accagcccgg ctgctacggc |
| 241 | cgcggataca cgccctcagg cccggcgctg cgcagcttgc ggaagctttc ccggacagac |
| 301 | tcgctgccag cggattggct gcgagcagcg ccaatctcac gttgcccccg ggcgaggcgg |
| 361 | gactcagtgc cgcgctctct gcacccgctc tgccgcgcgc gtgcgtgctg ggtgcgggtg |
| 421 | cgggtgcggg gttgggcctg cgcatcgggt gagacgctgg ctgcttgcgg ctagtggatg |
| 481 | gtaattgcct gcctcgcgct agcaggaagc tgctctgtta aaagcgaaaa tgaaacaatt |
| 541 | gcctgcggca acagttcgac tcctttcaag ttctcagatc atcacttcgg tggtcagtgt |
| 601 | tgtaaaagag cttattgaaa actccttgga tgctggtgcc acaagcgtag atgttaaact |
| 661 | ggagaactat ggatttgata aaattgaggt gcgagataac ggggagggta tcaaggctgt |
| 721 | tgatgcacct gtaatggcaa tgaagtacta cacctcaaaa ataaatagtc atgaagatct |
| 781 | tgaaaatttg acaacttacg gttttcgtgg agaagccttg gggtcaattt gttgtatagc |
| 841 | tgaggtttta attacaacaa gaacggctgc tgataatttt agcacccagt atgttttaga |
| 901 | tggcagtggc cacatacttt ctcagaaacc ttcacatctt ggtcaaggta caactgtaac |
| 961 | tgctttaaga ttatttaaga atctacctgt aagaaagcag ttttactcaa ctgcaaaaaa |
| 1021 | atgtaaagat gaaataaaaa agatccaaga tctcctcatg agctttggta tccttaaacc |
| 1081 | tgacttaagg attgtctttg tacataacaa ggcagttatt tggcagaaaa gcagagtatc |
| 1141 | agatcacaag atggctctca tgtcagttct ggggactgct gttatgaaca atatggaatc |
| 1201 | ctttcagtac cactctgaag aatctcagat ttatctcagt ggatttcttc caaagtgtga |
| 1261 | tgcagaccac tctttcacta gtctttcaac accagaaaga agtttcatct tcataaacag |
| 1321 | tcgaccagta catcaaaaag atatcttaaa gttaatccga catcattaca atctgaaatg |
| 1381 | cctaaaggaa tctactcgtt tgtatcctgt tttctttctg aaaatcgatg ttcctacagc |
| 1441 | tgatgttgat gtaaatttaa caccagataa aagccaagta ttattacaaa ataaggaatc |
| 1501 | tgttttaatt gctcttgaaa atctgatgac gacttgttat ggaccattac ctagtacaaa |
| 1561 | ttcttatgaa aataataaaa cagatgtttc cgcagctgac atcgttctta gtaaaacagc |
| 1621 | agaaacagat gtgcttttta ataaagtgga atcatctgga aagaattatt caaatgttga |
| 1681 | tacttcagtc attccattcc aaaatgatat gcataatgat gaatctggaa aaaacactga |
| 1741 | tgattgttta aatcaccaga taagtattgg tgactttggt tatggtcatt gtagtagtga |
| 1801 | aatttctaac attgataaaa acactaagaa tgcatttcag gacatttcaa tgagtaatgt |
| 1861 | atcatgggag aactctcaga cggaatatag taaaacttgt tttataagtt ccgttaagca |
| 1921 | cacccagtca gaaaatggca ataaagacca tatagatgag agtggggaaa atgaggaaga |
| 1981 | agcaggtctt gaaaactctt cggaaatttc tgcagatgag tggagcaggg gaaatatact |
| 2041 | taaaaattca gtgggagaga atattgaacc tgtgaaaatt ttagtgcctg aaaaaagttt |
| 2101 | accatgtaaa gtaagtaata ataattatcc aatccctgaa caaatgaatc ttaatgaaga |
| 2161 | ttcatgtaac aaaaaatcaa atgtaataga taataaatct ggaaaagtta cagcttatga |
| 2221 | tttacttagc aatcgagtaa tcaagaaacc catgtcagca agtgctcttt ttgttcaaga |
| 2281 | tcatcgtcct cagtttctca tagaaaatcc taagactagt ttagaggatg caacactaca |
| 2341 | aattgaagaa ctgtggaaga cattgagtga agaggaaaaa ctgaaatatg aagagaaggc |
| 2401 | tactaaagac ttggaacgat acaatagtca aatgaagaga gccattgaac aggagtcaca |
| 2461 | aatgtcacta aaagatggca gaaaaaagat aaaacccacc agcgcatgga atttggccca |
| 2521 | gaagcacaag ttaaaaacct cattatctaa tcaaccaaaa cttgatgaac tccttcagtc |
| 2581 | ccaaattgaa aaaagaagga gtcaaaatat taaaatggta cagatcccct tttctatgaa |
| 2641 | aaacttaaaa ataaatttta agaaacaaaa caaagttgac ttagaagaga aggatgaacc |
| 2701 | ttgcttgatc cacaatctca ggtttcctga tgcatggcta atgacatcca aaacagaggt |
| 2761 | aatgttatta aatccatata gagtagaaga agccctgcta tttaaaagac ttcttgagaa |
| 2821 | tcataaactt cctgcagagc cactggaaaa gccaattatg ttaacagaga gtctttttaa |
| 2881 | tggatctcat tatttagacg ttttatataa aatgacagca gatgaccaaa gatacagtgg |
| 2941 | atcaacttac ctgtctgatc ctcgtcttac agcgaatggt ttcaagataa aattgatacc |
| 3001 | aggagtttca attactgaaa attacttgga aatagaagga atggctaatt gtctcccatt |
| 3061 | ctatggagta gcagatttaa aagaaattct taatgctata ttaaacagaa atgcaaagga |
| 3121 | agtttatgaa tgtagacctc gcaaagtgat aagttattta gagggagaag cagtgcgtct |
| 3181 | atccagacaa ttacccatgt acttatcaaa agaggacatc caagacatta tctacagaat |
| 3241 | gaagcaccag tttggaaatg aaattaaaga gtgtgttcat ggtcgcccat tttttcatca |
| 3301 | tttaacctat cttccagaaa ctacatgatt aaatatgttt aagaagatta gttaccattg |
| 3361 | aaattggttc tgtcataaaa cagcatgagt ctggttttaa attatctttg tattatgtgt |
| 3421 | cacatggtta ttttttaaat gaggattcac tgacttgttt ttatattgaa aaaagttcca |
| 3481 | cgtattgtag aaaacgtaaa taaactaata tagactattc aaaaaaaaaa aaaaaaaa |
| and/or | |
| MLH3 (mRNA NM_001040108.1; protein NP_001035197.1). | |
| (SEQ ID NO: 62) | |
| 1 | aacaactggt gcgcatgcgc actggtgtct cgcggcctgg cgcgccccct ccgaagcgca |
| 61 | tgctcgtggg cacgcacgag cctcaagatc caaggtgcgc gcgtcggcgt ccgaggcggt |
| 121 | tggtgtcgga gaatttgtta agcgggactc caggcaatta tttccagtca gagaaggaaa |
| 181 | ccagtgcctg gcattctcac catctttcta cctaccatga tcaagtgctt gtcagttgaa |
| 241 | gtacaagcca aattgcgttc tggtttggcc ataagctcct tgggccaatg tgttgaggaa |
| 301 | cttgccctca acagtattga tgctgaagca aaatgtgtgg ctgtcagggt gaatatggaa |
| 361 | accttccaag ttcaagtgat agacaatgga tttgggatgg ggagtgatga tgtagagaaa |
| 421 | gtgggaaatc gttatttcac cagtaaatgc cactcggtac aggacttgga gaatccaagg |
| 481 | ttttatggtt tccgaggaga ggccttggca aatattgctg acatggccag tgctgtggaa |
| 541 | atttcgtcca agaaaaacag gacaatgaaa acttttgtga aactgtttca gagtggaaaa |
| 601 | gccctgaaag cttgtgaagc tgatgtgact agagcaagcg ctgggactac tgtaacagtg |
| 661 | tataacctat tttaccagct tcctgtaagg aggaaatgca tggaccctag actggagttt |
| 721 | gagaaggtta ggcagagaat agaagctctc tcactcatgc acccttccat ttctttctct |
| 781 | ttgagaaatg atgtttctgg ttccatggtt cttcagctcc ctaaaaccaa agacgtatgt |
| 841 | tcccgatttt gtcaaattta tggattggga aagtcccaaa agctaagaga aataagtttt |
| 901 | aaatataaag agtttgagct tagtggctat atcagctctg aagcacatta caacaagaat |
| 961 | atgcagtttt tgtttgtgaa caaaagacta gttttaagga caaagctaca taaactcatt |
| 1021 | gactttttat taaggaaaga aagtattata tgcaagccaa agaatggtcc caccagtagg |
| 1081 | caaatgaatt caagtcttcg gcaccggtct accccagaac tctatggcat atatgtaatt |
| 1141 | aatgtgcagt gccaattctg tgagtatgat gtgtgcatgg agccagccaa aactctgatt |
| 1201 | gaatttcaga actgggacac tctcttgttt tgcattcagg aaggagtgaa aatgttttta |
| 1261 | aagcaagaaa aattatttgt ggaattatca ggtgaggata ttaaggaatt tagtgaagat |
| 1321 | aatggtttta gtttatttga tgctactctt cagaagcgtg tgacttccga tgagaggagc |
| 1381 | aatttccagg aagcatgtaa taatatttta gattcctatg agatgtttaa tttgcagtca |
| 1441 | aaagctgtga aaagaaaaac tactgcagaa aacgtaaaca cacagagttc tagggattca |
| 1501 | gaagctacca gaaaaaatac aaatgatgca tttttgtaca tttatgaatc aggtggtcca |
| 1561 | ggccatagca aaatgacaga gccatcttta caaaacaaag acagctcttg ctcagaatca |
| 1621 | aagatgttag aacaagagac aattgtagca tcagaagctg gagaaaatga gaaacataaa |
| 1681 | aaatctttcc tggaacatag ctctttagaa aatccgtgtg gaaccagttt agaaatgttt |
| 1741 | ttaagccctt ttcagacacc atgtcacttt gaggagagtg ggcaggatct agaaatatgg |
| 1801 | aaagaaagta ctactgttaa tggcatggct gccaacatct tgaaaaataa tagaattcag |
| 1861 | aatcaaccaa agagatttaa agatgctact gaagtgggat gccagcctct gccttttgca |
| 1921 | acaacattat ggggagtaca tagtgctcag acagagaaag agaaaaaaaa agaatctagc |
| 1981 | aattgtggaa gaagaaatgt ttttagttat gggcgagtta aattatgttc cactggcttt |
| 2041 | ataactcatg tagtacaaaa tgaaaaaact aaatcaactg aaacagaaca ttcatttaaa |
| 2101 | aattatgtta gacctggtcc cacacgtgcc caagaaacat ttggaaatag aacacgtcat |
| 2161 | tcagttgaaa ctccagacat caaagattta gccagcactt taagtaaaga atctggtcaa |
| 2221 | ttgcccaaca aaaaaaattg cagaacgaat ataagttatg ggctagagaa tgaacctaca |
| 2281 | gcaacttata caatgttttc tgcttttcag gaaggtagca aaaaatcaca aacagattgc |
| 2341 | atattatctg atacatcccc ctctttcccc tggtatagac acgtttccaa tgatagtagg |
| 2401 | aaaacagata aattaattgg tttctccaaa ccaatcgtcc gtaagaagct aagcttgagt |
| 2461 | tcacagctag gatctttaga gaagtttaag aggcaatatg ggaaggttga aaatcctctg |
| 2521 | gatacagaag tagaggaaag taatggagtc actaccaatc tcagtcttca agttgaacct |
| 2581 | gacattctgc tgaaggacaa gaaccgctta gagaactctg atgtttgtaa aatcactact |
| 2641 | atggagcata gtgattcaga tagtagttgt caaccagcaa gccacatcct taactcagag |
| 2701 | aagtttccat tctccaagga tgaagattgt ttagaacaac agatgcctag tttgagagaa |
| 2761 | agtcctatga ccctgaagga gttatctctc tttaatagaa aacctttgga ccttgagaag |
| 2821 | tcatctgaat cactagcctc taaattatcc agactgaagg gttccgaaag agaaactcaa |
| 2881 | acaatgggga tgatgagtcg ttttaatgaa cttccaaatt cagattccag taggaaagac |
| 2941 | agcaagttgt gcagtgtgtt aacacaagat ttttgtatgt tatttaacaa caagcatgaa |
| 3001 | aaaacagaga atggtgtcat cccaacatca gattctgcca cacaggataa ttcctttaat |
| 3061 | aaaaatagta aaacacattc taacagcaat acaacagaga actgtgtgat atcagaaact |
| 3121 | cctttggtat tgccctataa taattctaaa gttaccggta aagattcaga tgttcttatc |
| 3181 | agagcctcag aacaacagat aggaagtctt gactctccca gtggaatgtt aatgaatccg |
| 3241 | gtagaagatg ccacaggtga ccaaaatgga atttgttttc agagtgagga atctaaagca |
| 3301 | agagcttgtt ctgaaactga agagtcaaac acgtgttgtt cagattggca gcggcatttc |
| 3361 | gatgtagccc tgggaagaat ggtttatgtc aacaaaatga ctggactcag cacattcatt |
| 3421 | gccccaactg aggacattca ggctgcttgt actaaagacc tgacaactgt ggctgtggat |
| 3481 | gttgtacttg agaatgggtc tcagtacagg tgtcaacctt ttagaagcga ccttgttctt |
| 3541 | cctttccttc cgagagctcg agcagagagg actgtgatga gacaggataa cagagatact |
| 3601 | gtggatgata ctgttagtag cgaatcgctt cagtctttgt tctcagaatg ggacaatcca |
| 3661 | gtatttgccc gttatccaga ggttgctgtt gatgtaagca gtggccaggc tgagagctta |
| 3721 | gcagttaaaa ttcacaacat cttgtatccc tatcgtttca ccaaaggaat gattcattca |
| 3781 | atgcaggttc tccagcaagt agataacaag tttattgcct gtttgatgag cactaagact |
| 3841 | gaagagaatg gcgaggcagg tgggaacctg ctcgtgctgg tggatcagca cgctgcccat |
| 3901 | gagcgtatac gtctggagca gcttatcatt gattcctacg agaagcaaca ggcacaaggc |
| 3961 | tctggtcgga aaaaattact gtcttctact ctaattcctc cgctagagat aacagtgaca |
| 4021 | gaggaacaaa ggagactctt atggtgttac cacaaaaatc tggaagatct gggccttgaa |
| 4081 | tttgtatttc cagacactag tgattctctg gtccttgtgg gaaaagtacc actatgtttt |
| 4141 | gtggaaagag aagccaatga acttcggaga ggaagatcta ctgtgaccaa gagtattgtg |
| 4201 | gaggaattta tccgagaaca actggagcta ctccagacca ccggaggcat ccaagggaca |
| 4261 | ttgccactga ctgtccagaa ggtgttggca tcccaagcct gccatggggc cattaagttt |
| 4321 | aatgatggcc tgagcttaca ggaaagttgc cgccttattg aagctctgtc ctcatgccag |
| 4381 | ctgccattcc agtgtgctca cgggagacct tctatgctgc cgttagctga catagaccac |
| 4441 | ttggaacagg aaaaacagat taaacccaac ctcactaaac ttcgcaaaat ggcccaggcc |
| 4501 | tggcgtctct ttggaaaagc agagtgtgat acaaggcaga gcctgcagca atccatgcct |
| 4561 | ccctgtgagc caccatgaga acagaatcac tggtctaaaa ggaacaaagg gatgttcact |
| 4621 | gtatgcctct gagcagagag cagcagcagc aggtaccagc acggccctga ctgaatcagc |
| 4681 | ccagtgtccc tgagcagctt agacagcagg gctctctgta tcagtctttc ttgagcagat |
| 4741 | gattccccta gttgagtagc cagatgaaat tcaagcctaa agacaattca ttcatttgca |
| 4801 | tccatgggca cagaaggttg ctatatagta tctacctttt gctacttatt taatgataaa |
| 4861 | atttaatgac agtttgattg gttgcttggt ttgttatttg aagggtgtga tttttgtttt |
| 4921 | tgtacagttt tttttcaagc ttcacatttg cgtgtatcta attcagctga tgctcaagtc |
| 4981 | caaggggtag tctgccttcc caggctgccc ccagggtttc tgcactggtc ccctcttttc |
| 5041 | ccttcagtct tcttcacttc cctatgctgc tgcttcatgt gctacatctc agacttaaag |
| 5101 | agtttctcta ctacagtgaa aacattctct agggtctttc atcaggcctt tagttatttt |
| 5161 | agggataaaa actattgata aaaaggacaa ggatagaaca gagaaaattt aaagtcctgt |
| 5221 | tccgggtttt ttgttatgtt ttctttaaaa actcagagac tgatgttcaa tatcccaaac |
| 5281 | cagtaaaatg gtgaaaatac tatgagcttg ttttttaaaa tatgattttt tttggtactt |
| 5341 | tataaagtat ctctttatgt gaaagcaatt gtcatatcaa aacacagcat acatacgttc |
| 5401 | aacctaacca aatatcttta cactttttct ttcaggagac aagggttctt tgggtccctt |
| 5461 | tcaaacggta tcttggtgtt attacattat gcctatctat tgcccttata atatcacttg |
| 5521 | ggaccaggac tgatcgttct gcaaatgctt gttatgccat tctcaatcta tttttcccgc |
| 5581 | accttttcac atgatttgtg gttaatagga ctcaacagac taaaattgca tagtagaaaa |
| 5641 | aaaatgcaaa aagccagctg gtaatgttta ttgcaactgg ggtgctatac aattagtaag |
| 5701 | atgatgcaat gagaatttct acttttgtat ttcctgacca gcctgctcaa agtggctttt |
| 5761 | atatcaattg aatgattttc ctcatttttt aatacaggaa accaattcgt gctcatggaa |
| 5821 | gaaaagttcc tttgccagca gccttgaagt gaatcttaca ggagcaatga aagtattgca |
| 5881 | ttcattagcg tctgccccag agaaggttca gagaaaacct tcacttgttt tcaaggggat |
| 5941 | ccttgtagat ttacgtaatt ggaatcctga agaacaggcc ctactgtcta aaaaatggct |
| 6001 | tttattcttc taaatacata taaacggatg ttttatagat gggaagacat gaccttagaa |
| 6061 | aggagagagt tttcagagga tttgccaggc tgtcaggggc tctgcctcca ggcccagtgt |
| 6121 | ggcagtgtgg cctcagggcc tccgcctccc tgcttgaggg ctgcatggag gccaactgtc |
| 6181 | ctgggagttg taaaaatctt ttaaggccag accaatttga gggattttaa aaagtgtctc |
| 6241 | agtgcctctt atgatttcag aaggttttgc tatatgtaat cccaactact gttttcttga |
| 6301 | gagtagcaga ggattagaaa aagtcctcca taaattatgt aaccggcctt cctgactagc |
| 6361 | ctgactcaag caatgtaaga gataattatt ctgttttcat aatttataag tgtgggggca |
| 6421 | tgcctcagca taaaaacaac ctattaggga aaaatatcta atagattacc tttatcgcct |
| 6481 | gttagggttt tatgttgttt ttaactcaga tgccataaga acaaagatac atgtaattta |
| 6541 | taatagtaat cattaatacc tatattgtgc tttaaggttt acaaaataat ttttctcata |
| 6601 | ctttatctta gtttagtttc ttgacagtcc atgaggtaag gtggtagctt tatcaccatt |
| 6661 | ttacaaagtg ggaaacgaag gttcctctta ggaacctagt tgtcaccttt gtataataaa |
| 6721 | acttcgaagc tcggagctgt taactggttt gctgaaggct tagctgtaag agccagaatt |
| 6781 | cagacccagg tctgagtgac ttcaaactgc acagtccttc ccattattac ccatatgcta |
| 6841 | tcccttatat ttttaattta ttaggaattc attcatttat aaacttggtg attcaccttt |
| 6901 | attagattct ggtcgctgaa ggctttagta acttcagagt aaaacttgag agatgagatg |
| 6961 | taaaatgcag ccattcttga gagttccttt ttctgtaaca ttcatcaaca cttcattgag |
| 7021 | aagtgaaggt tcctatggct gtctctacct tcaagaggct tagctttagt cactgagaaa |
| 7081 | gacaaggaaa ctaatgatag aatatagtag cttcttctgg cgttaggtat cacagagtca |
| 7141 | cagctagtta cagctagccc tttattattg aaagaagagg agctagcagt cccactatca |
| 7201 | gaattaagac tagagatggt aataggagct agtatcagaa aagcttaagg caaagcataa |
| 7261 | agtgtaggct agaatgaagc tggagaatgg ggagggggct tgggtaacat ccagaacctg |
| 7321 | gctggggacc tggaactaca tgagatgtaa gaatggagag gttctagcag tcagaggtca |
| 7381 | ggtacaaatg aacagctggg atctgcgcat ggcagacagt gaaaaaaccc aggcaagcaa |
| 7441 | aatggtcaga gcagaaaggg gcccaaggcc acgttcttga gatgtggagg gggctgagga |
| 7501 | agccacgcca agtaaggaca gatgcagctc agcagttcct agcgagccct gacaagccag |
| 7561 | ctcagctgaa gcttcgggtg ggagccagtc atggcacagt ggagtgaagg aagagcagtt |
| 7621 | tcaggcaccc aaaacctgac ccccacgacc tgttttccac ctgaagagcc acccattcca |
| 7681 | tccaaaccct tggcaaaagt ctgctaacag agagaaccgg ccagtatgct ggccagtcgc |
| 7741 | gatcatgcct gtctttaccc tctaagctga agctgctcat caacggtgag atggcaaaaa |
| 7801 | ggtgggtcca gaagagggga aaagaaggga gtctgtgaaa acaaaatgct gaagaatctg |
| 7861 | catcaaataa acccttcctt ccttcctttt tccttcaaaa aaaaaaaaaa a |

These sequences are hereby incorporated by reference in their entirety.

Inhibition of Gene Expression and/or Protein Activity

The expression of RNA and/or protein can be inhibited by a variety of methods. For example, RNA expression can be inhibited by “knockout” procedures or “knockdown” procedures. Generally, with a “knockout,” expression of the gene in an organism or cell is eliminated by engineering the gene to be inoperative or removed. In a “knockdown,” the expression of the gene may not be completely inhibited, but only partially inhibited, such as with antisense (antisense molecules interact with complementary strands of nucleic acids, modifying expression of genes), ribozyme, RNAi or shRNA technology.

As used herein, the term “antisense oligonucleotide” or antisense nucleic acid means a nucleic acid polymer, at least a portion of which is complementary to a nucleic acid that is present in a normal cell or in an affected cell. “Antisense” refers particularly to the nucleic acid sequence of the non-coding strand of a double-stranded DNA molecule encoding a protein, or to a sequence that is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

As used herein, the terms “complementary” or “complementarity” are used in reference to polynucleotides (i.e., a sequence of nucleotides) related by the base pairing rules. For example, for the sequence “A G T,” is complementary to the sequence “T C A.”

In RNA interference (RNAi), double-stranded RNA is synthesized with a sequence complementary to a gene of interest and introduced into a cell or organism, where it is recognized as exogenous genetic material and activates the RNAi pathway. A small hairpin RNA or short hairpin RNA (shRNA) is a sequence of RNA that makes a tight hairpin turn that can be used to silence gene expression via RNA interference. Small interfering RNA (siRNA), sometimes known as short interfering RNA or silencing RNA, is a class of double-stranded RNA molecules that play a variety of roles in biology. Most notably, siRNA is involved in the RNA interference (RNAi) pathway, where it interferes with the expression of a specific gene(s). siRNA can be used to modify expression of the genes mentioned herein.

An inhibitor of expression or protein activity can be any inhibitor of the preselected gene/protein (such as those described herein), for example, the inhibitor can be an antibody that specifically binds to the protein, a nucleic acid that inhibits expression (e.g., a nucleic acid that can hybridize to the DNA or mRNA), or a compound (e.g., small molecule).

### Expression/Overexpression or Increase Protein Activity

In one embodiment, the genes and proteins discussed herein are overexpressed so as produce, for example, a preselected protein in amounts greater than normally found in that cell type. Nucleic acids encoding proteins described herein can be used for recombinant expression of the proteins, for example, by operably-linking the nucleic acid to an expression control sequence within an expression vector, which can be introduced into a host cell for expression of the encoded peptide.

As used herein, the term “operably linked” means that a nucleic acid and an expression control sequence are positioned in such a way that the expression control sequence directs expression of the nucleic acid under appropriate culture conditions and when the appropriate molecules such as RNA transcriptional proteins are bound to the expression control sequence.

The term “expression control sequence” refers to a nucleic acid sequence sufficient to direct the transcription of another nucleic acid sequence that is operably linked to the expression control sequence to produce an RNA transcript.

An “expression vector” is a nucleic acid molecule capable of transporting and/or allowing for the expression of another nucleic acid to which it has been linked. Expression vectors contain appropriate expression control sequences that direct expression of a nucleic acid that is operably linked to the expression control sequence to produce a transcript. The product of that expression is referred to as a messenger ribose nucleic acid (mRNA) transcript. The expression vector may also include other sequences such as enhancer sequences, synthetic introns, and polyadenylation and transcriptional termination sequences to improve or optimize expression of the nucleic acid encoding the protein.

Nucleic acids encoding proteins can be incorporated into bacterial, viral, insect, yeast or mammalian expression vectors so that they are operably-linked to expression control sequences such as bacterial, viral, insect, yeast or mammalian promoters (and/or enhancers).

Nucleic acid molecules or expression cassette that encode proteins may be introduced to a vector, e.g., a plasmid or viral vector, which optionally includes a selectable marker gene, and the vector introduced to a cell of interest, for example, a bacterial, yeast or mammalian host cell.

Expression cassettes or vectors containing nucleic acids encoding proteins can be introduced into bacterial, insect, yeast or mammalian host cells for expression using conventional methods including, without limitation, transformation, transduction and transfection (calcium-mediated transformation, electroporation, microinjection, lipofection, particle bombardment and the like).

The expression of the encoded protein may be controlled by any promoter capable of expression in prokaryotic cells or eukaryotic cells. Examples of prokaryotic promoters that can be used include, but are not limited to, SP6, T7, T5, tac, bla, trp, gal, lac or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV. In some embodiments, the expression vector is the pRG5 vector (Coppi et al., Appl. Environ. Microbiol. 67: 3180-87 (2001)); Leang et al., BMC Genomics 10, 331 (2009).

Construction of suitable vectors can employ standard ligation techniques. Isolated plasmids or DNA fragments are cleaved, tailored, and re-ligated in the form desired to generate the plasmids required.

### Culture Conditions

During and after the gene targeting process, the cells can be cultured in culture medium that is established in the art and commercially available from the American Type Culture Collection (ATCC), Invitrogen and other companies. Such media include, but are not limited to, Dulbecco's modified Eagle's medium (DMEM), DMEM F12 medium, Eagle's minimum essential medium, F-12K medium, Iscove's modified Dulbecco's medium, knockout D-MEM, RPMI-1640 medium, or McCoy's 5A medium. It is within the skill of one in the art to modify or modulate concentrations of media and/or media supplements as needed for the cells used. It will also be apparent that many media are available as low-glucose formulations, with or without sodium pyruvate.

Also contemplated is supplementation of cell culture medium with mammalian sera. Sera often contain cellular factors and components that are needed for cell viability. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, rat serum (RS), serum replacements, and bovine embryonic fluid. It is understood that sera can be heat-inactivated at 55-65° C. if deemed needed to inactivate components of the complement cascade. Modulation of serum concentrations, or withdrawal of serum from the culture medium can also be used to promote survival of one or more desired cell types. In one embodiment, the cells are cultured in the presence of FBS/or serum specific for the species cell type. For example, cells can be isolated and/or expanded with total serum (e.g., FBS) concentrations of about 0.5% to about 5% or greater including about 5% to about 15%.

Concentrations of serum can be determined empirically.

Additional supplements can also be used to supply the cells with trace elements for optimal growth and expansion. Such supplements include insulin, transferrin, sodium selenium, and combinations thereof. These components can be included in a salt solution such as, but not limited to, Hanks' Balanced Salt Solution™ (HBSS), Earle's Salt Solution™, antioxidant supplements, MCDB-201™ supplements, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids. Many cell culture media already contain amino acids; however some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

Antibiotics are also typically used in cell culture to mitigate bacterial, mycoplasmal, and fungal contamination. Typically, antibiotics or anti-mycotic compounds used are mixtures of penicillin/streptomycin, but can also include, but are not limited to, amphotericin (Fungizone™) ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be advantageously used in cell culture and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine. β-mercaptoethanol can also be supplemented in cell culture media.

Lipids and lipid carriers can also be used to supplement cell culture media, depending on the type of cell and the fate of the differentiated cell. Such lipids and carriers can include, but are not limited to cyclodextrin (α, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others. Albumin can similarly be used in fatty-acid free formulation.

Cells in culture can be maintained either in suspension or attached to a solid support, such as extracellular matrix components and synthetic or biopolymers. Cells often require additional factors that encourage their attachment to a solid support (e.g., attachment factors) such as type I, type II, and type IV collagen, concanavalin A, chondroitin sulfate, fibronectin, “superfibronectin” and/or fibronectin-like polymers, gelatin, laminin, poly-D and poly-L-lysine, Matrigel™, thrombospondin, and/or vitronectin.

### EXAMPLES

The following examples are provided in order to demonstrate and further illustrate certain embodiments and aspects of the present invention and are not to be construed as limiting the scope thereof.

### Example I

### Materials and Methods

### Targeting Vector Construction

Construction of the pAAV-HPRT exon 3 Neo or pAAV-HPRT exon 3 Puro targeting vector containing multiple restriction endonuclease SNPs and sequences that created 9 bp hairpins in each homology arm was carried out in a multi-step process utilizing PCR, restriction enzyme digestion and subsequent DNA ligation as well as site-directed mutagenesis. Briefly, HCT116 genomic DNA was used as template for PCR reactions to create homology arms flanking exon 3 of the HPRT locus. Primers used to create either the left or right homology arms include HPRT.3 NdeI LF 5′-ATACATACGCGGCCGCTCAAGCACTGGCTATGCATGTATACCATATGCAAAAG-3′ (SEQ IDNO:1), HPRT.3 SacII LR 5′-TTATCCGCGGTGGAGCTCCAGCTTTTGTTCCCTTTAGTCAGGAATTTAATAGAAAGTTTCAT AC-3′ (SEQ IDNO:2) and HPRT.3 KpnI RF 5′-TTATGGTACCCAATTCGCCCTATAGTGAGTCGTATTACTTGCTTTCATTTCACTTGGTTACAG TG-3′ (SEQ IDNO:3), HPRT.3 SbfI RR 5′-ATACATACGCGGCCGCTTAAATGGCTGCCCAATCACCTGCAGGATTGATG-3′ (SEQ IDNO:4). Fusion PCR was then performed using the PCR-generated left and right homology arms along with a PvuI restriction enzyme-digested fragment from the pNeDaKO Neo vector to create a NotI-digestible vector fragment that was subsequently ligated into pAAV-MCS. The resulting plasmid was then subjected to eight rounds of mutagenesis using the Quikchange Site Directed Mutagenesis Kit (Stratagene) to incorporate six SNPs creating an EcoRI, NcoI, and AseI restriction site in the 5′-homology arm and a SacI and XbaI restriction site in the 3′ homology arm as well as a hairpin containing a 9 bp stem with a 4 bp loop in each homology arm. The primer pairs used are listed in Table 1.

TABLE 1
PCR Primers Used In the Construction of
HPRT Targeting Vectors

| | | | Distance |
|---|---|---|---|
| Site | Primer | | From |
| Created: | Name: | Primer Sequence: | Heterology: |
| 5′- | | | |
| Homology | | | |
| Arm: | | | |
| NdeI | HPRT.3 | 5′-ATACATACGCG | 1126 bp |
| | NdeI LF | GCCGCTCAAGCACTG | |
| | | GCTATGCATGTATACC | |
| | | ATATGCAAAAG-3′ | |
| | | (SEQ ID NO: 19) | |
| EcoRI | HPRT.3 | 5′-GTTCAGCTTTATT | 868 bp |
| | EcoRI F | CAAGTGGAATTCCTG | |
| | | GGTCAAGGGG-3′ | |
| | | (SEQ ID NO: 20) | |
| | HPRT.3 | 5′-CCCCTTGACCCA | |
| | EcoRI R | GGAATTCCACTTGAAT | |
| | | AAAGCTGAAC-3′ | |
| | | (SEQ ID NO: 21) | |
| Hairpin | HPRT.3 | 5′-TTCAAGCGATCC | 632 bp |
| | LHPF | TTCCACCCCAGCTAC | |
| | | GTGAGTAGCTGGGAC | |
| | | TATAG-3′ | |
| | | (SEQ ID NO: 22) | |
| | HPRT.3 | 5′-CTATAGTCCCAGC | |
| | LHPR | TACTCACGTAGCTGG | |
| | | GGTGGAAGGATCGCT | |
| | | TGAA-3′ | |
| | | (SEQ ID NO: 23) | |
| NcoI | HPRT.3 | 5′-AGGGTTCGCCA | 547 bp |
| | NcoIF | TGGTACCCAGCCTCC | |
| | | C-3′ | |
| | | (SEQ ID NO: 24) | |
| | HPRT.3 | 5′-GGGAGGCTGGG | |
| | NcoIR | TACCATGGCGAACCC | |
| | | T-3′ | |
| | | (SEQ ID NO: 25) | |
| AseI | HPRT.3 | 5′-TTAATGACTAAGA | 33 bp |
| | AseIF | GGTGTTTGTTATAAAG | |
| | | ATTAATGTATGAAACT | |
| | | TTCTATTAAATTCC-3′ | |
| | | (SEQ ID NO: 26) | |
| | HPRT.3 | 5′-GGAATTTAATAGA | |
| | AseIR | AAGTTTCATACATTAA | |
| | | TCTTTATAACAAACAC | |
| | | CTCTTAGTCATTAA-3′ | |
| | | (SEQ ID NO: 27) | |
| 3′- | | | |
| Homology | | | |
| Arm: | | | |
| NdeI | HPRT.3 | 5′-ACTTGGTTACAGT | 40 bp |
| | SspIF | GAGATTTTTCTAAAAT | |
| | | ATTCACTAGTACTTTA | |
| | | CATCAAAG-3′ | |
| | | (SEQ ID NO: 28) | |
| | HPRT.3 | 5′-CTTTGATGTAAAG | |
| | Sspir | TACTAGTGAATATTTT | |
| | | AGAAAAATCTCACTG | |
| | | TAACCAAGT-3′ | |
| | | (SEQ ID NO: 29) | |
| XbaI | HPRT.3 | 5′-TTTTTATTACTTT | 261 bp |
| | XbaIF | AGACTATTGTTTCTAG | |
| | | ATTTTATGTTGGGTTG | |
| | | GTATTTCCTG-3′ | |
| | | (SEQ ID NO: 30) | |
| | HPRT.3 | 5′-CAGGAAATACCA | |
| | XbaIR | ACCCAACATAAAATCT | |
| | | AGAAACAATAGTCTA | |
| | | AAGTAATAAAAA-3′ | |
| | | (SEQ ID NO: 31) | |
| Hairpin | HPRT.3 | 5′-CTGCAGGTATACT | 400 bp |
| | RHPF | CAAGAGAGTATACCG | |
| | | CACCAGAAACCACTT | |
| | | TTG-3′ | |
| | | (SEQ ID NO: 32) | |
| | HPRT.3 | 5′-CAAAAGTGGTTT | |
| | RHPR | CTGGTGCGGTATACT | |
| | | CTCTTGAGTATACCTG | |
| | | CAG-3′ | |
| | | (SEQ ID NO: 33) | |
| SacI | HPRT.3 | 5′-GAGTCAAAAGTC | 571 bp |
| | SacIF | CTTTGGAGCTCGGTT | |
| | | TGACAAATAAGGTG-3′ | |
| | | (SEQ ID NO: 34) | |
| | HPRT.3 | 5′-CACCTTATTTGTC | |
| | SacIR | AAACCGAGCTCCAAA | |
| | | GGACTTTTGACTC-3′ | |
| | | (SEQ ID NO: 35) | |
| SbfI | HPRT.3 | 5′-ATACATACGCGGC | 841 bp |
| | SbfI RR | CGCTTAAATGGCTGC | |
| | | CCAATCACCTGCAGG | |
| | | ATTGATG-3′ | |
| | | (SEQ ID NO: 36) | |

### Viral Production

rAAV-HPRT NENASSXS+2HP Exon 3 Neo or rAAV-HPRT NENASSXS+2HP Exon 3 Puro virus was generated using a triple transfection strategy in which the targeting vector (8 μg) was mixed with pAAV-RC and pAAV-helper (8 μg each) and was then transfected onto 4×10⁶ AAV-293 cells using Lipofectamine 2000 (Invitrogen). Virus was isolated from the AAV-293 cells 48 hr later by scraping the cells into 1 ml of media followed by three rounds of freeze/thawing in liquid nitrogen (40).

### Infections

HCT116 cells were grown to ˜70-80% confluency on 6-well tissue culture plates. Fresh media (1 ml) was added at least 30 min prior to the addition of virus. At that time, the required amount of virus was added drop-wise to the plates. The cells and virus were allowed to incubate for 2 hr before adding back more media (3 ml). When using the version of the virus containing the neomycin drug resistance marker, infected cells were allowed to grow for 2 days before they were sub-cultured by trypsinization and plated at 2×10⁶ cells per 10 cm plates under 1 mg/ml G418 and 5 μg/ml 6-thioguanine selection. When using the version of the vector containing the puromycin resistance gene, the cells were plated first in media containing 1 μg/ml puromycin for 4-5 days to allow drug resistant colonies to form. The puromycin-containing media was then removed and replaced with media containing 5 μg/ml 6-thioguanine. In addition, single drug selection (either G418 or puromycin) was used to select for randomly targeted clones. This was done in order to demonstrate that the clones produced by correct targeting had used a different mechanism during integration of the viral genome compared to the randomly targeted clones.

### Isolation of Genomic DNA and PCR

Genomic DNA for PCR was isolated using the PureGene DNA Purification Kit (Qiagen). Cells were harvested from confluent wells of a 24-well tissue culture plate. DNA was resuspended in 50 μl hydration solution, 2 μl of which was used for each PCR reaction using 2× Failsafe PCR Buffer E (Epicentre) and a laboratory-prepared stock of Taq polymerase. For HPRT exon 3 targeting events, correct targeting was determined for both the 5′- and 3′-homology arms. For the 5′-homology arm the primer pair HPRT.3 EF 5′-TTGAATGCTTGCATTGTATGTCTGGC-3′ (SEQ ID NO:5) and NeoR2 5′-AAAGCGCCTCCCCTACCCGGTAGG-3′ (SEQ ID NO:6) was used while the primer pair ZeoF1 5′-ACGTGACCCTGTTCATCAGC-3′ (SEQ ID NO:7) and HPRT.3 ER 5′-AAACAAGTCTTTAATTCAAGCAAGAC-3′ (SEQ ID NO:8) was used for the 3′-homology arm analysis.

### SNP Analysis

In order to determine which restriction endonuclease SNPs were incorporated into the target cells' genome from the viral DNA during integration, each PCR product produced from correctly targeted clones was used for multiple restriction enzyme digests. Typically 5 μl of each 25 μl PCR reaction was first electrophoresed on a 1% agarose gel to determine if there was enough product for digestion. Subsequently, 5 μl from samples containing enough product of the correct size were then used in 20 μl restriction enzyme digests, utilizing restriction enzymes whose sites were generated, or inactivated, by the point mutations found in the targeting vector. For the 5′-homology arm, NdeI, EcoRI, NcoI, and AseI digests were performed, while for the 3′-homology arm the restriction enzymes SspI, SacI, Xbak and SbfI were used. In addition, the creation and retention of the mutations that created the 5′-hairpin fortuitously produced a point mutation that abolished a BbvCI site. So correctly targeted clones whose PCR products were digestible by BbvCI corresponded to events in which the 5′ hairpin was not incorporated. In addition, however, the vast majority of both 5′- and 3′-PCR products were also subjected to DNA sequencing to determine unequivocally the loss or retention of the hairpin sequences, as well as to confirm the restriction enzyme SNP retention. The 5′-homology arm was sequenced using HPRTLIntR2 (5′-CCACGTAACACATCCTTTGCCCTC-3′; SEQ ID NO:9) while the 3′-homology arm was sequenced using HPRT.3 SspIF.

### Primers Used in the Construction of a RAD52-Null Cell Line

The primers are coded—underlined: genomic sequence; bold: restriction sites; italics: LoxP site; black: junk sequence or spacers):

LarmF_NotI:
(SEQ ID NO: 10)
ATACATACGCGGCCGCGAGCAGTACCTAGTACGTTGAC
LarmR_SpeI:
(SEQ ID NO: 11)
GGACTAGTCATGCGGCTACTTATGTATTCTG
RarmF_XhoI:
(SEQ ID NO: 12)
CCAGCTCGAGGGCCAGAAGGTAGGAGAA
RarmR_NotI:
(SEQ ID NO: 13)
ATACATACGCGGCCGCGGCTGAGACACAACTCTG
CasF_SpeI:
(SEQ ID NO: 14)
GACTAGTATAACTTCGTATAGCATACATTATACGAAGTTATGCATGGCTG
AACAAGATGG
CasR_XhoI:
(SEQ ID NO: 15)
CCAGCTCGAGCATACATATGCACAGTGGTAC
Larm_intF:
(SEQ ID NO: 16)
CACTGCTATGATGCCTAATG
ExpF:
(SEQ ID NO: 17)
GAGCAGTACCTAGTACGTTGAC
NeoR:
(SEQ ID NO: 18)
AGGTGAGATGACAGGAGAT

### Results

### Analysis of Recombinant Products

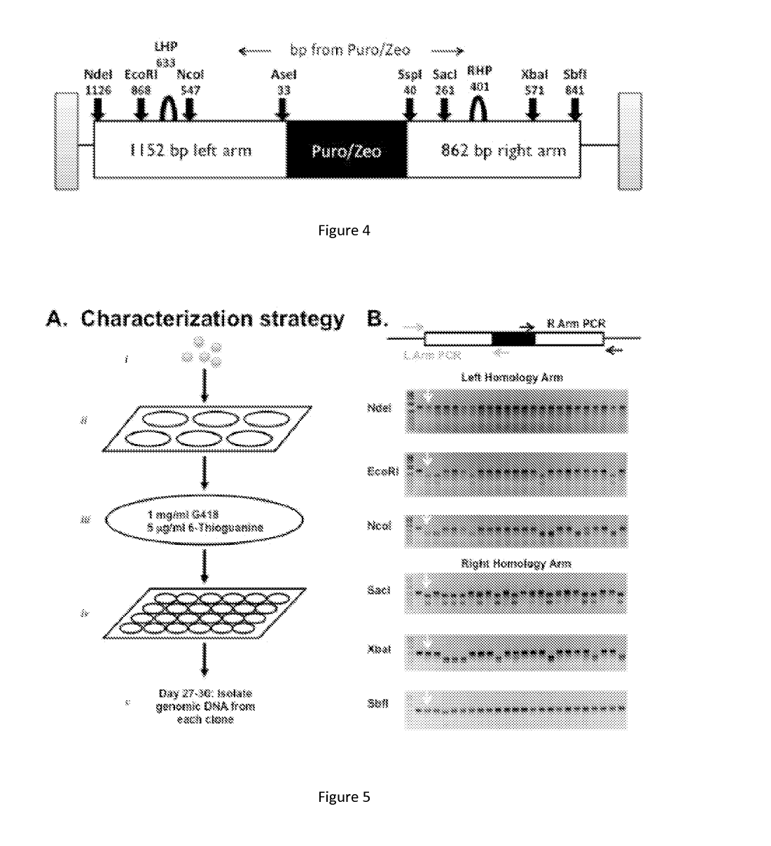

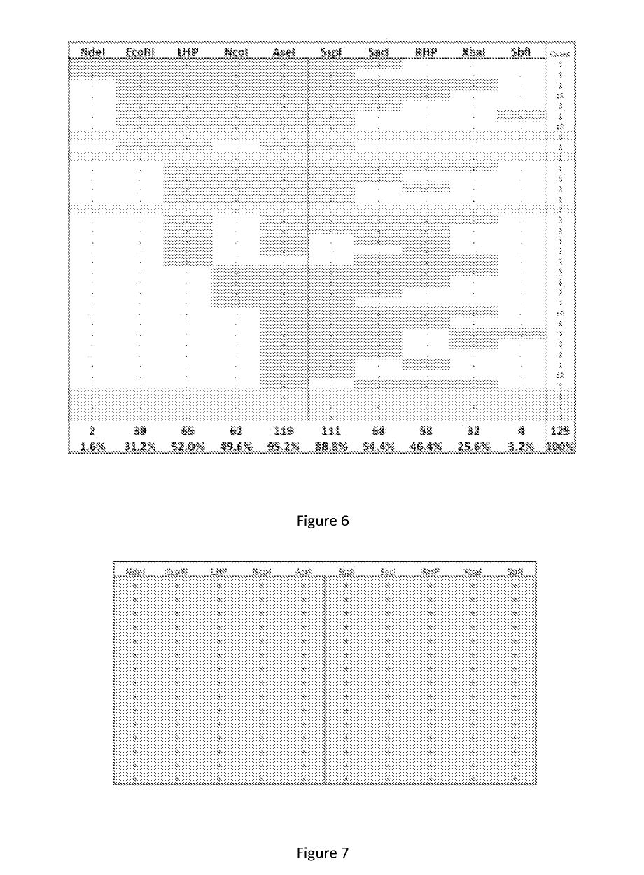

A two-ended, ends-out dsDNA mechanism of gene targeting predicts trans products of recombination (FIG. 2), whereas a ssDNA assimilation model predicts cis products (FIG. 3). To test these predictions experimentally for rAAV-mediated gene targeting, a rAAV gene-targeting vector capable of replacing exon 3 of the HPRT (hypoxanthine phosphoribosyl transferase) gene was constructed. In addition, 4 SNPs and a 22 bp palindromic sequence (consisting of a 9 bp stem with 4 unpaired nucleotides at the tip) were built into each arm of the vector (FIG. 4). This vector was then used to infect the human colorectal carcinoma HCT116 cell line (28). HCT116 cells were chosen because they have been used by a large number of independent laboratories to carry out successful gene targeting experiments (7, 11, 44, 57). The HPRT locus was chosen as a target because it resides on the X chromosome and thus, in a male-derived cell line like HCT116, HPRT is hemizygous and requires only one round of gene targeting to produce a null phenotype. The absence of HPRT enzymatic activity confers resistance to a drug, 6-thioguanine (53), making the identification of correctly targeted clones by drug selection quite simple. Thus, HCT116 cells were infected with the HPRT NENASSXS+2HP vector (FIG. 5A, i) and subsequently placed under double drug selection: one drug for the uptake of the virus (usually G418 or puromycin) and 6-thioguanine to select for the loss of HPRT expression (FIG. 5A, iii). Individual clones were expanded and about a month later, genomic DNA was prepared (FIG. 5A, v). PCR amplification of the region corresponding to each targeting arm (FIG. 5B) was carried out and the resulting PCR products subjected to restriction enzyme digestion analysis. A representative experiment (from many) of the resulting ethidium bromide-stained agarose gels are shown and a single clone is highlighted that has incorporated the EcoRI, NcoI and SacI restriction enzyme sites from the virus, but did not acquire the NdeIi, XbaIi or SbfI sites. In addition, almost every one of the PCR-amplified arms was subjected to DNA sequencing to confirm the restriction enzyme analyses and to identify the presence or absence of the viral donor palindromic sequences (data not shown). A total of 125 HCT116 HPRT NENASSXS+2HP correctly (i.e., puromycin-resistant, 6-thioguanine resistant and diagnostic PCR positive) targeted clones were analyzed. Of these clones, 14 (11.2%) displayed SNP retention patterns (i.e., trans) consistent with the double-strand invasion model of gene targeting (FIG. 6). The remaining 111 clones (88.8%) retained SNPs on both the 5′- and 3′-homology arms (FIG. 6) consistent with a ssDNA assimilation model of rAAV integration. As a control, 15 randomly targeted clones were obtained (i.e., puromycin-resistant, 6-thioguanine sensitive) and subjected to a similar PCR analysis. In 100% of these cases (15/15) all of the SNPs were observed to have been acquired at the site of integration (FIG. 7) in stark contrast to the pattern(s) observed for correctly targeted events (FIG. 6). These data are consistent with the random viral integration events probably occurring with dsDNA through the process of NHEJ (non-homologous end joining) as has been previously hypothesized (18, 29). Altogether, it is concluded that approximately 90% of rAAV-mediated gene targeting events are not easily explained by a two-ended, ends-out dsDNA mechanism of gene targeting, but are instead more consistent with a ssDNA assimilation model. Moreover, whatever mechanism of recombination is used for the correctly targeted events, it appears to be completely distinct than the mechanism utilized for the random integration of exactly the same donor viral vector.

Additional features of the recombination events were also evident. As well as informing about which strategy the virus predominately utilizes to assimilate its genome, it was observed that the farther the SNP was located from the drug resistance marker (which presumably forms a very large (˜2 kb) region of heterology), the more likely it was to be lost during viral integration (FIGS. 6 & 8). This feature presumably results from these sites being used as sites of crossing over between the viral and chromosomal sequences. Indeed, a 50% probability of retention for any given SNP was observed only about 300 bp distant from the drug resistant marker (FIGS. 6 & 8) suggesting that even if the entire arm (˜1 kb long) of the gene targeting vector is base paired with the chromosome (e.g., as depicted in FIG. 3) that crossing over generally occurs over much shorter intervals. Such a characteristic is, once again, probably more consistent with a ssDNA assimilation rather than a two-ended, ends-out dsDNA mechanism, where large regions of homology generated during Holliday Junction migration are common (1).

To address the impact of the cellular MMR status on rAAV-mediated gene targeting, identical experiments to those described above, were carried out using a derivative HCT116 cell line in which the MLH1 mutation (which exists in the parental HCT116 cell line and which renders it MMR-defective) has been corrected by a targeted knock-in (Horizon Discovery). Although the number of data points to date is not as large as have been obtained with the parental cell line, no difference in the cis/trans configurations for targeted clones (FIG. 9A), the retention of SNPs in the randomly targeted clones (FIG. 9B) or the retention of SNPs as a function of their position along the rAAV targeting arms (FIG. 9C) were observed. From these data, it appears as if the MMR repair status of the cell does not impact the overall mechanism of rAAV-mediated gene targeting (but see below for an exception).

The Genetics of rAAV-Mediated Gene Targeting

A genetic methodology was also utilized to address the mechanism of rAAV-mediated gene targeting. If a two-ended, ends-out dsDNA mechanism is in fact utilized during gene targeting, a straightforward prediction would be that mutations in canonical HR genes should reduce or ablate subsequent gene targeting events. Unfortunately, many of the HR genes encode essential factors and only a handful of mutants are available for use. Nonetheless, this prediction is generally borne out. The best example of this comes from RAD54B, one of the two mammalian RAD54 homologs (FIG. 1, v). When the RAD54B gene was inactivated in HCT116 cells, subsequent gene targeting events using standard dsDNA transfection methodologies were reduced to undetectable levels and reduced by at least an order of magnitude at two independent loci (30). When the XRCC3 (a Rad51 paralog) gene was inactivated, a small decrease (of only 15 to 30%) in gene targeting was subsequently observed at two independent loci using standard dsDNA transfection methodologies (60). The lack of an effect similar to what was observed in RAD54B-null cells was explained by the likelihood that an additional Rad51 paralog (XRCC3 is one of 7 Rad51 genes in humans; (52)) was likely compensating for the absence of XRCC3. A third cell line, this one defective in Mus81 (15), has been described. Mus81 is a component of one of the three human resolvases (FIG. 1, vi; (58)) and it would be expected to impact significantly on canonical two-ended, ends-out dsDNA recombination, although some redundancy between the resolvases is apparent (58). No subsequent gene targeting experiments, however, have been described using this cell line so its effect is still hypothetical.

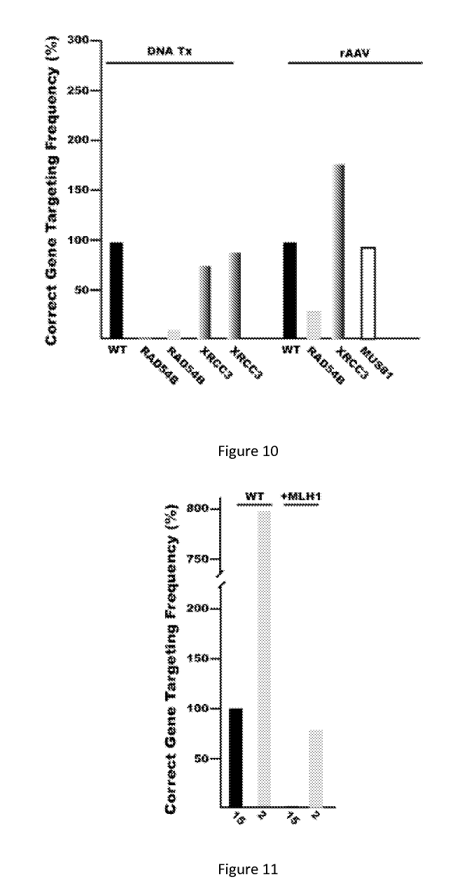

To test the impact of loss-of-function mutations on rAAV-mediated gene targeting, rAAV was used to target either the CCR5 (chemokine C-C receptor gene 5) or HPRT loci in RAD54B-null cells and the HPRT locus in XRCC3-null and Mus81-null cell lines. Whereas correctly targeted clones arising from the transfection of dsDNA were virtually ablated in Rad54B null cells, rAVV-mediated gene targeting, albeit reduced, was less affected (25% of the wild-type frequency; FIG. 10). Interestingly whereas dsDNA-mediated gene targeting was slightly affected in XRCC3-null cells, the frequency of rAAV-mediated gene targeting actually increased over 1.9 fold (FIG. 10). Similarly, rAAV-mediated gene targeting was just as efficient in Mus81-null cells as compared to the parental HCT116 cell line (FIG. 10). As noted above, similar data for gene targeting facilitated by dsDNA transfection are not available for Mus81-null cells so a direct comparison for this technology is currently not possible. Nonetheless, these data paint a compelling picture in which mutations in canonical HR genes that, at least in the case of RAD54B, deleteriously affect canonical two-ended, ends-out dsDNA gene targeting while appearing to have much less effect or actually improve the efficiency of rAAV-mediated gene targeting. These observations suggest that rAAV-mediated gene targeting does not occur by the commonly accepted mechanism of gene targeting and are more consistent with a ssDNA annealing/assimilation pathway.

To address this hypothesis, the impact of a functional MMR system on the frequency of rAAV-mediated gene targeting was also determined. For these experiments, two vectors were utilized, which were otherwise identical except that one contained 15 independent mismatches with the target sequence (HPRT) and one that had only two mismatches. When these vectors were used with the parental HCT116 cell line (which is MMR defective) a striking difference was nonetheless observed. The vector containing only 2 mismatches targeted 8 times better than the vector containing 15 mismatches (FIG. 11). This effect was greatly exacerbated in the MLH1-complemented cell line where the vector containing only 2 mismatches targeted 11-times less well than in the parental (MMR-deficient) cell line and the vector containing 15 mismatches targeted over 100-fold less well at virtually undetectable levels (FIG. 11). These experimental data demonstrate that even though the presence or absence of a functioning MMR system in a cell doesn't impact the mechanism of gene targeting per se (FIGS. 6 through 9) it does significantly affects the frequency with which such events occur (FIG. 11). These data support the contention that rAAV proceeds through a ssDNA annealing/assimilation mechanism and that a functioning MMR system impedes this process.

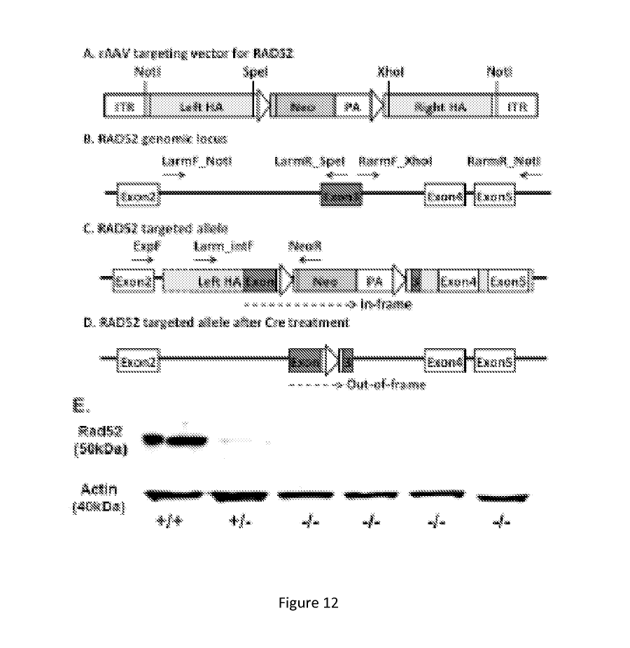

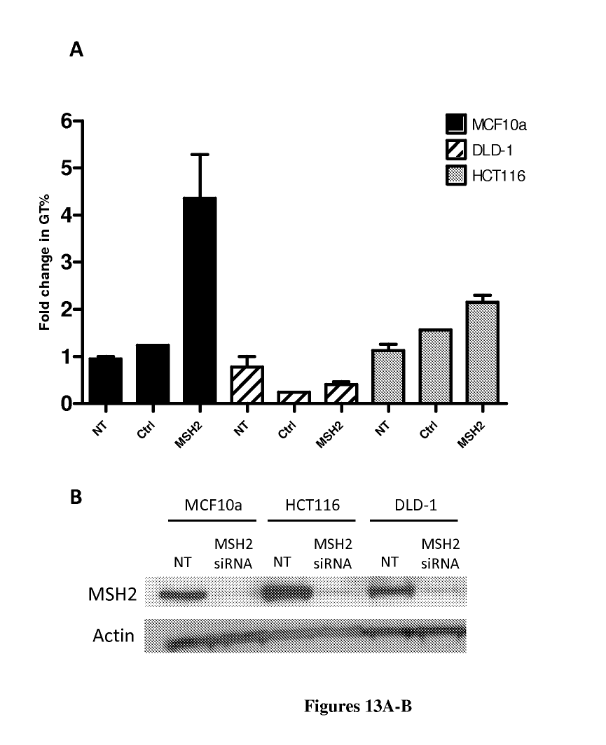

RAD52 is a 419 amino acid protein encoded by 12 exons on human chromosome 12. Because an internal translational start was found in-frame in the latter half of exon 3, which might drive the translation of a truncated ORF, both ORFs were disrupted by engineering a frame-shift mutation shortly after that ATG in exon 3. A rAAV gene targeting was constructed, which contained a selection cassette flanked by left and right homology arms of ˜1500 bp (FIG. 12A). The ˜1100 bp selection cassette was composed of a promoterless Neo resistance gene (NEO) and a poly-adenylation sequence (pA), flanked by loxP sites. The homology arms were cloned by PCR from HCT116 genomic DNA with the designated primers (FIG. 12B). The selection cassette was amplified with primers CasF_SpeI and CasR_XhoI from the pSEPT vector as described (54). The vector was assembled by digesting the homology arms and selection cassette with the designated restriction enzymes (FIG. 12A), and ligating with NotI-restricted AAV-MCS backbone as described (39). After virus infection, the cells were grown with 1 mg/mL G418 for 14 days. The G418-resistant clones were then analyzed by diagnostic PCRs (FIG. 12C; Larm_intF and NeoR for viral integration, ExpF and NeoR for correct targeting). In the correctly-targeted clones, the promoterless NEO cassette was fused to the 3′ end of exon 3 in-frame, and the expression of the fusion protein was driven by the endogenous RAD52 promoter. When the selection cassette was removed by the addition of AdCre, the remaining LoxP site resulted in a frameshift for the rest of the ORF (FIG. 12D). In total, two rounds of targeting were performed to remove both alleles of RAD52. The first round of targeting gave a targeting frequency of 57%: out of 64 G418 resistant clones, 49 clones contained the viral DNA and 28 of them were correctly targeted. In the second round, 31 correctly targeted clones were recovered from 63 G418-resistant clones (49.2% targeting), and 15 of them were targeted to the second allele. The expression level of RAD52 became undetectable after two rounds of targeting confirming the authenticity of the clones (FIG. 12E). These clones are currently being utilized to carry out subsequent rAAV-mediated gene targeting studies where it is anticipated that the absence of RAD52 will greatly restrict the ability of rAAV to correctly target.

### Discussion

This is the first demonstration of applying time-honored technologies for measuring and characterizing genetic recombination to rAAV-mediated gene targeting. These studies, both molecular and genetic, have provided a compelling and surprising conclusion that rAAV-mediated gene targeting does not involve the canonical HR pathway utilized in lower eukaryotes (12, 22). Instead, our studies demonstrate that rAAV can utilize a subpathway of HR, termed single-strand annealing/assimilation (50).

In the intervening 13 years since the discovery that rAAV could be utilized to perform gene targeting in human somatic cells (41) there has been little progress in determining how rAAV performs gene targeting. Thus, almost all previous models of gene targeting have required the presence of dsDNA ends: either on the incoming donor DNA, on the endogenous recipient chromosome, or on both. For example, this is the strategy of gene targeting mediated by ZFNs: “make a DSB on a chromosome and the gene targeting factors will come” (59). Indeed, it is even true that making DSBs in the chromosome will greatly increase rAAV-mediated gene targeting (35, 36). However, applying these models to normal rAAV-mediated gene targeting was difficult from the beginning. Thus, the frequency of rAAV-mediated gene targeting is so high (14, 21) that it cannot be accounted for by the presumed frequency of spontaneous DSBs in human cells (about 15/per cell/per day; (9)). Consequently, it was widely assumed that if the dsDNA ends were not on the chromosome, they must be coming from rAAV and it seemed likely that some dsDNA replicative form of rAAV (42) was the actual intermediate for gene targeting. The data presented herein calls this model sharply into question. By generating a rAAV vector with SNPs imbedded within the homology arms, it has been demonstrated herein that the vast majority (89%) of gene-targeted products are more consistent with having been generated by a ssDNA annealing/assimilation pathway.

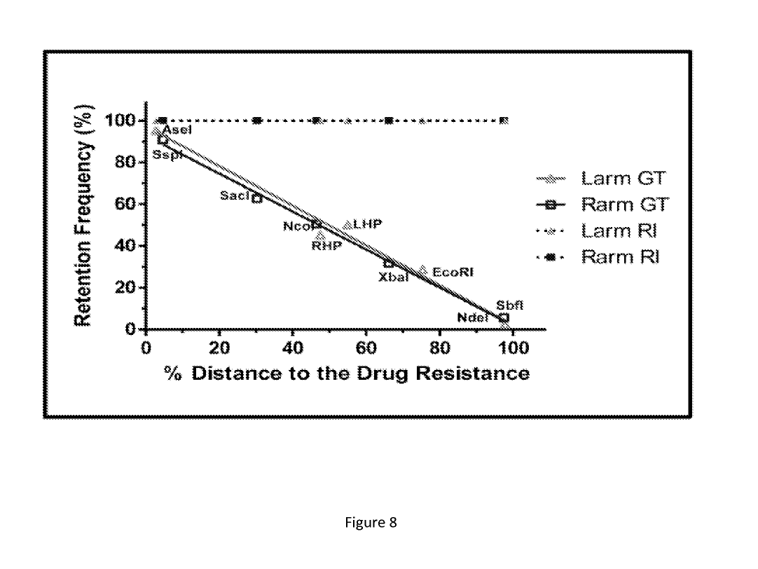

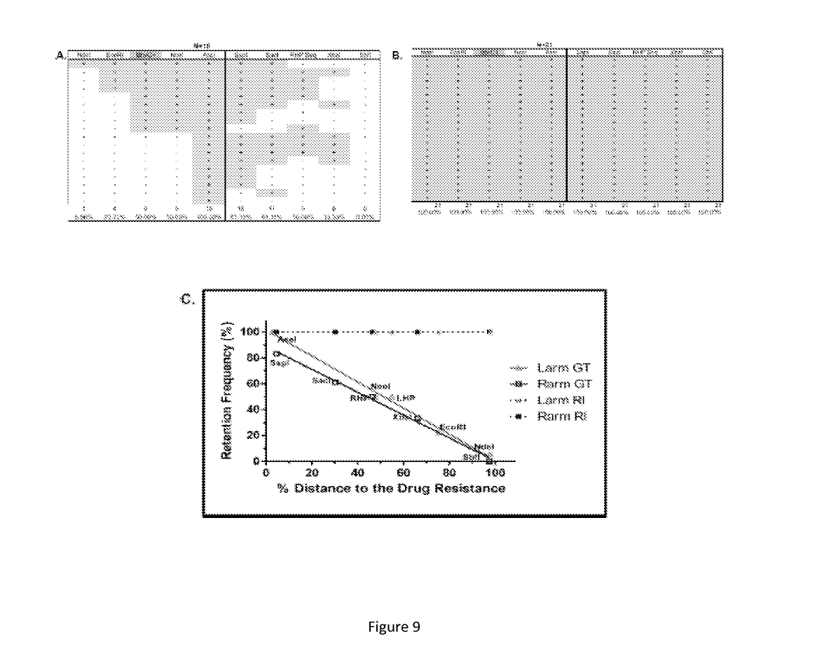

The genetic studies provide complementary data to the molecular studies. Thus, it would appear that mutations in HR genes should disrupt canonical gene targeting. However, due to the essential nature of many of the HR factors the relevant experiments are technically very difficult to carry out and therefore have not yet been reported in the literature. The most compelling example comes from RAD54B, which is one of the two RAD54 paralogs in human cells. When this gene is disrupted, subsequent canonical dsDNA-mediated gene targeting is ablated or severely crippled (30). In striking comparison, in gene targeting studies carried out at two independent loci in Rad54B-null cells, rAAV-mediated gene targeting was only mildly reduced (FIG. 8). Perhaps even more compelling was the observation that in XRCC3- and Mus81-null cell lines the relative frequency of rAAV-mediated gene targeting actually increased. Perhaps, by disrupting the major HR pathway, these mutations have freed up other HR factors to carry out the HR subpathway of ssDNA annealing/assimilation more efficiently.

### Example II

### rAAV Targeted Knockout of Artemis in HCT116 Cells

### Introduction

Artemis (occasionally referred to as SNMC1 (Sensitive to Nitrogen Mustard C1)) was originally identified as a gene that, when mutated (Moshous et al.), was responsible for a subset of human patients afflicted with RS-SCID (Radiation-Sensitive, Severe Combined Immune Deficiency) (Nicolas et al.). Subsequent biochemical characterization of Artemis demonstrated that it was a DNA-PKcs-(DNA-dependent Protein Kinase complex Catalytic Subunit) dependent, structure specific nuclease (Kurosawa and Adachi). Artemis' role in causing SCID when it is mutated is well understood. Artemis has hairpin resolving nuclease activity and hairpin resolution is an intermediate step in V(D)J (Variable(Diversity)Joining) recombination, a lymphoid-restricted, site-specific recombination process in the development of the human immune system (Ma et al.). Thus, when Artemis is mutated, hairpinned V(D)J recombination intermediates accumulate and no functional B- or T-cells can be generated (Rooney et al.). Artemis' role in causing RS when it is mutated is less well understood, but presumably is due to the lack of resolution of hairpinned-like DNA structures that may be generated during ionizing radiation exposure. Interestingly, although Artemis is a member of a family of structure-specific nucleases consisting of at least five members (Cattell et al. and Yan et al.), these proteins have apparently evolved distinct properties since the expression of the other four nucleases is not sufficient to compensate for the loss of Artemis (Moshous et al.).

Although Artemis has been investigated predominately for its roles in V(D)J recombination and DNA repair, it has also been implicated in rAAV infections, but not in rAAV-mediated gene targeting. Studies carried out in either DNA-PKcs- or Artemis-deficient mouse cells showed that rAAV replication intermediates containing unprocessed hairpinned ITRs (Inverted Terminal Repeats) accumulated (Inagaki et al.) in a manner highly reminiscent of what had been observed for hairpinned V(D)J recombination intermediates (Rooney et al.). In a somewhat parallel study, the DNA locations where rAAV randomly integrates in mouse cells were identified and sequenced. These sites were biased toward palindromic (i.e., potentially hairpinned) sequences (Inagaki et al.). Thus, a model based upon these results is that Artemis may be required to process either the viral ITRs or genomic hairpins (or both) to facilitate random rAAV integrations. The bias towards random integrations at genomic palindromic sequences was not observed when a similar experiment using AAV was carried out in human somatic cells (Miller et al.).

To experimentally test the hypothesis that Artemis may regulate the frequency of rAAV-mediated gene targeting, using rAAV-mediated gene targeting technology, a human somatic cell line that no longer expresses Artemis was generated. The frequency of subsequent rAAV-mediated gene targeting in this cell line was enhanced. This observation suggests that Artemis normally suppresses rAAV-mediated gene targeting.

### Materials and Methods

### Targeting Vector Construction

Construction of the pAAV-Artemis exon 2 Neo or pAAV-Artemis exon 2 Puro targeting vectors was carried out by PCR followed by restriction enzyme digestion and subsequent DNA ligation (Kohli et al.). Briefly, HCT116 genomic DNA was used as a template for PCR reactions to create homology arms flanking exon 2 of the Artemis locus. Primers used to create either the left or right homology arms include ART2F: 5′-ATACATACGCGGCCGCGAGCCACCATGTCCAACT GGTTTAG-3′ (SEQ ID NO:37); ART2 SacIIR: TTATCCGCGGTGGAGCTCCAG CTTTTGTTCCCTTTAGAAAAGAACAAAAACTCATGAATATG-3′ (SEQ ID NO:38); ART2 KpnIF: 5′-ATGGTACCCAATTCGCCCTATAGTGAGTCGTAT TACTATTTTGCTACTTGTGTTTTTAAG-3′ (SEQ ID NO:39); and ART 2R: 5′-ATACATACGCGGCCGCGTCAATAAGTAAATACAAATAAAGTAATAAAAAATTATTGGC-3′ (SEQ ID NO:40). Fusion PCR was then performed using the PCR-generated left and right homology arms along with a PvuI restriction enzyme fragment derived from the pNeDaKO vector to create a NotI digestible vector fragment that was subsequently ligated into pAAV-MCS. In addition to pAAV-Artemis exon 2 Neo, pAAV-Artemis exon 2 Puro was also created. This was achieved using the original pAAV-Artemis exon 2 Neo vector and swapping out the drug selection cassettes. Briefly, a puromycin selection cassette from an engineered pNeDaKO Puro plasmid was removed using restriction enzyme digestion with SpeI and KpnI. This DNA fragment was then ligated to the SpeI/KpnI pAAV-Artemis exon 2 homology arm-containing fragment to generate pAAV-Artemis exon 2 Puro.

### Virus Production

rAAV-Artemis Exon 2 Neo virus was generated using a triple transfection strategy in which the targeting vector (8 μg) was mixed with pAAV-RC and pAAV-helper (8 μg each) and was then transfected into 4×10⁶ AAV-293 cells using Lipofectamine 2000 (Invitrogen). Virus was isolated from the AAV-293 cells 48 hr later by scraping the cells into 1 ml media followed by three rounds of freeze/thawing in liquid nitrogen (Khan et al. and Kohli et al.).

### Infections

HCT116 cells were grown to ˜70-80% confluency on 6-well tissue culture plates. Fresh media (1 ml) was added at least 30 min prior to the addition of virus. At that time, the required amount of virus was added drop-wise to the plates. The cells and virus were allowed to incubate for 2 hr before adding back more media (3 ml). The infected cells were allowed to grow for 2 days before they were trypsinized and plated at 2000 cells per well of 96-well plates under the appropriate drug selection (Ruis et al.).

### Isolation of Genomic DNA and PCR

Genomic DNA for PCR was isolated using the PureGene DNA purification kit (Qiagen). Cells were harvested from confluent wells of a 24-well tissue culture plate. DNA was resuspended in 50 μl hydration solution, 2 μl of which was used for each PCR reaction. For Artemis exon 2 heterozygous targeting events, a control PCR was performed using the 3′-side of the targeted locus using the primer set RArmF: 5′-CGCCCTATAGTGAGTCGTATTAC-3′ (SEQ ID NO:41) and ART2R: 5′-ATACATACGCGGCCGCGTCAATAAGTAAATACAAATAAAGTAATAA AAAATTATTGGC-3′ (SEQ ID NO:42). Correct targeting was determined by PCR using RArmF and ART2R1 5′-GTCACAGGTGACCAAAAAAAATTACTG-3′ (SEQ ID NO:43) primers. For the second round of targeting, PCR was performed again using the 3′-side of the targeted locus, however, the vector-specific primer was replaced with NeoF1: 5′-TTCTTGACGAGTTCTTCTGAGGGGATCAATTC-3′(SEQ ID NO:44). For the third round of targeting, a control PCR was performed for the 5′-side of the targeted locus using the primer set ART2F-1: 5′-GAGCCACC ATGTCCAACTGGTTTAG-3′ (SEQ ID NO:45) and NeoR2: 5′-AAAGCGCCTCC CCTACCCGGTAGG-3′ (SEQ ID NO:46). Correct targeting was determined by using ART2EF: 5′-ACTGGGTCTAATGATGGCCACACGAC-3′ (SEQ ID NO:47). The null status was determined using a pair of Artemis exon 2 flanking primers that produce different sized products when amplified from an exon 2-containing allele or a Lox P site-containing allele. This PCR was performed using ART2 5′F: 5′-CCCTTGGGCTAAGGAATCCTCTGG-3′ (SEQ ID NO:48) and ART2 3′R: 5′-AATGTTTGCTTAAAAACACAAGTAGC-3′ (SEQ ID NO:49).

### Gene Targeting Strategy

In order to knock out the first allele of Artemis, the rAAV-Artemis exon 2 Neo virus was used. The relative targeting frequency was 3/176 or 1.7%. Once a correctly targeted clone was identified, the neomycin selection cassette was removed by Cre recombination (Ruis et al.). Briefly, the cells were transfected with the PML-Cre plasmid using Lipofectamine LTX after which they were plated at limited dilutions onto 10 cm dishes and allowed to form colonies. Approximately 2 weeks later, individual colonies were characterized for confirmation of the loss of one allele of Artemis exon 2 bp PCR and for G418 sensitivity. The second round of targeting was methodology was identical to that used in the first round. 14 independent correctly gene targeted clones were produced from 1700 drug resistant clones (0.82% gene targeting frequency). Although at this time it was expected that some of these clones would by null for Artemis, PCR analysis using primers flanking exon 2 of Artemis, as well as an exon 2-specific primer, showed that Artemis in the HCT116 cell line was at least triploid. This was perhaps not surprising since there is a large duplication on the q arm of one chromosome 10 (Masramon et al.); the same chromosome where the Artemis locus resides (Moshous et al.). After another round of Cre treatment, this time using CMV AdCre virus (Wang et al.), a third round of gene targeting was performed using rAAV-Artemis exon 2 Puro virus. Five correctly targeted clones were obtained out of 120 drug-resistant clones for a relative targeting frequency of 4.2%. Two of these clones (clone 15 and clone 18) were determined to be null for Artemis exon 2 based on PCR using exon 2 flanking primers ART2 5′F and ART2 3′R.

### Gene Targeting Efficiency in Artemis Null Cells

rAAV XRCC4 exon 4 Neo virus was used for viral infection as described above. G418 resistant single colonies (50) were isolated from 96-well plates and expanded to 24-well plates for isolation of genomic DNA. The harvested DNA was then subjected to PCR to determine correct targeting using the primer pair RArmF and XRCC4.4 ER2: 5′-GCCAAATAACACTAGATGTTAGGAAC-3′ (SEQ ID NO:50). To confirm the presence of the integrated vector the primer pair RArmF and XRCC4.4 RR: 5′-ATACATACGCGGCCGCGTCTATACAGAGCAATCAC AATGG-3′ (SEQ ID NO:51) was used.

### Results

In order to determine if the loss of Artemis confers higher relative gene targeting frequencies, the HCT116 Artemis exon 2^{-/-/-} (subclone 15.1) cells were used in an experiment in which XRCC4 exon 4 was targeted. Fifty drug-resistant clones that were also PCR-positive for rAAV were obtained. Seven of the 50 clones tested were determined to be correctly targeted; resulting in a relative gene targeting frequency of 14.0%. Gene targeting at this locus in the parental cell line was 22 correctly targeted clones from 2026 clones analyzed (compilation of three independent experiments) for a gene targeting frequency of 1.1%. Thus, the absence of Artemis resulted in a 12.7-fold (14.0% versus 1.1%) stimulation in the relative correct gene targeting frequency.

### Discussion

In Artemis-deficient human somatic cell lines, the frequency of relative rAAV-mediated gene targeting is improved by over an order of magnitude.

### Example III

### MSH2 Knockdown—FIG. 13

Cell Culture

The human colon cancer cell lines HCT116 and DLD-1 were obtained from the American Type culture collection (ATCC) and maintained in RPMI 1640 media (Invitrogen) supplemented with 10% heat inactivated calf serum (Sigma), 2 mM L-glutamine, 100 U/ml penicillin and 100 U/ml streptomycin (Invitrogen). HEK293T cells were obtained from ATCC and cultured in DMEM F-12 Nutrient mix (HAM) (Invitrogen) supplemented with 10% heat inactivated calf serum, 100 U/ml penicillin and 100 U/ml streptomycin. The MFC10a cell line was obtained from ATCC and maintained in DMEM:F12 media with L-glutamine (Invitrogen) supplemented with 5% Horse Serum, 0.1 μg/ml cholera toxin, 20 ng/ml human EGF, 10 μg/ml Insulin) and 500 ng/ml hydrocortisone (Sigma), 100 U/ml penicillin and 100 U/ml streptomycin (Invitrogen). For drug selection, the media was supplemented with G418 (sigma) at a final concentration of 0.3 mg/ml, 0.1 mg/ml or 0.35 mg/ml for HCT116, MCF10a or DLD-1 cells respectively. All cell lines were grown at 37° C. in a humidified incubator with 5% CO₂.

Targeting Vector Construction and Virus Production

The rAAV BRAF V600E targeting vector was generated by DNA synthesis of the homology arms and selection cassettes (Genscript, NJ USA). The synthesized fragment was cloned by restriction enzyme digestion and ligation into the pAAV-MCS backbone plasmid (Agilent) between the two copies of the AAV-2 ITR sequences to facilitate viral packaging.

Infectious rAAV was generated by co-transfection of the targeting vector and the pDG helper plasmid (PlasmidFactory GmbH, Germany) into HEK293T cells using lipofectamine LTX reagent (Invitrogen) following the manufacturer's protocol. Virus was harvested 72 hours after transfection. Briefly, media was collected from the T75 flask and the HEK293T cells were washed in 3 ml of phosphate-buffered saline (Invitrogen), 2 ml of TrypLE Express dissociation reagent (Invitrogen) was added to the flask which was incubated for 5 minutes at 37° C. Dissociated cells were harvested and the collected media and cell suspension centrifuged for 5 minutes at 1000×g. Cell pellets and clarified supernatants were stored at −80° C., before being subjected to three freeze-thaw cycles. Each cycle consisted of 10 min freeze in a dry ice/ethanol bath, and 10 min thaw in a 37° C. water bath. The lysate was then clarified by centrifugation at 1000×g for 30 minutes. Approximately 2500 units of Benzonase nuclease (Sigma) was added to the clarified supernatant which was incubated at 37° C. for a further 30 minutes. Virus was purified from the treated supernatant using the AAV Purification ViraKit (ViraPur, CA USA) according to the manufacturer's instructions. Aliquots of purified virus were stored at −80° C. until use.

The titer of purified viral stocks was measured by Q-PCR. Briefly, 5 μl of purified virus was treated with amplification grade DNase I (Sigma) for 30 minutes at 37° C., followed by treatment with proteinase K (Sigma) for 1 hour at 56° C. Dilutions of the treated virus were compared to dilutions of standard virus stocks (known titers) in Q-PCR assays using oligonucleotide primers and FAM-dye labeled probes (Applied Biosystems) specific for the neomycin resistance selection cassette.

siRNA Transfection and rAAV Infection

HCT116, DLD-1 and MCF10a cells were seeded at a density of 1.6×10⁵ cells in a T25 culture flask (BD). The following day, cells were transfected with either 20 nM of MSH2 siRNA (Sigma, cat#4392420) or 60 nM of a scrambled negative control siRNA (Sigma, cat#4390843) using Lipofectamine RNAimax reagent (Invitrogen) following the manufacturers protocol. The transfection solution was incubated with the cells for 6 hours and then replaced with culture media. Cells were cultured for a further 48 hours before being harvested, counted and reseeded at a density of 1.6×105 cells in a T25 culture flask to which the purified BRAF V600E rAAV was added at an multiplicity of infection (MOI) of 100,000 genome copies/virus particles per cell. Cells were incubated in the presence of virus for a further 72 hours before media was replaced and supplemented with G418 at the appropriate concentration. Cells were cultured under selection for a further two weeks.

Digital Droplet PCR (ddPCR) Screening Genomic DNA (gDNA)

Cells were harvested and gDNA extracted using the Maxwell 16 research system (Promega) following the manufacturers protocol. DNA concentrations were quantified using a Nanodrop spectrophotometer (Thermo Scientific). The gDNA was analyzed by ddPCR to measure the ratio of BRAF V600E locus-specific targeting events versus non-targeted BRAF alleles from the pool of cells. This ratio indicates the proportion of correctly targeted cells within the infected pool and can be expressed as a fold change between the siRNA treated and untreated controls to demonstrate the effect on gene targeting efficiency that MSH2 knockdown is having. A first round PCR was performed using a forward primer situated outside of the left homology arm (5′-GTGTAGGAGGGGAGCATTGA-3′; SEQ ID NO:56) and a reverse primer (5′-AGCATCTCAGGGCCAAAAAT-3′; SEQ ID NO:52) situated within the left homology arm, downstream of the V600E mutation. PCR reactions were performed with GoTaq Hot start Polymerase (Promega) using the conditions specified by the manufacturer. Using 10 ng template DNA, reactions were performed in 50 μl total volumes in 96-well plates using the following cycling conditions: 1 cycle of 94° C. for 3 minutes; 20 cycles of 94° C. for 30 seconds, 62° C. for 30 seconds, 72° C. for 90 seconds; 1 cycle of 72° C. for 5 minutes. Amplified PCR products were diluted 1:5000 in water and 10 μl then used in a second round ddPCR reaction in a 20 μl final volume. The ddPCR reactions were performed on the Bio-Rad QX100 system following the manufacturer's protocol. Using the PCR products from the first round PCR as a template, DNA primers and fluorescent TaqMan probes (Invitrogen) were used to amplify and quantify the number of alleles with the non-targeted BRAF V600 DNA sequence and the number of alleles with the targeted V600E sequence. Primer and probe sequences used in the ddPCR are as follows; forward: 5-CATGAAGACCTCACAGTAAAAATAGGTGAT-3′; Reverse: 5′-TGGGACCCACTCCATCGA-3′ (SEQ ID NO:53); VIC conjugated probe: 5′-CTAGCTACAGTGAAATC-3′ (SEQ ID NO:54); FAM conjugated probe: 5′ TAGCTACAGAGAAATC-3′ (SEQ ID NO:55). The data acquired was analyzed on QuantaSoft Droplet Digital PCR software (QuantaLife).

### Example IV

### MSH2 Knockdown—FIG. 13

MLH1 Expression and rAAV-Mediated Gene Targeting.

Introduction

Recombinant adeno-associated virus (rAAV) facilitates high-efficiency gene targeting in mammalian cells. It also holds promise for gene therapies of inherited diseases. Despite its wide applications in laboratorial and clinical settings, the mechanism of rAAV gene targeting remains obscure. Here, it is demonstrated that mismatches between the donor and recipient DNAs and the mismatch repair (MMR) status of the recipient cell affect the frequency of rAAV-mediated gene targeting. These findings will facilitate the development of safer and more efficient gene therapies.

Materials and Methods:

Cell Culture:

The human HCT116 cell line and its MLH1-complemented derivative were cultured in McCoy's 5A medium supplemented with 10% FBS, 2 mM L-glutamine, 100 U/ml penicillin and 100 U/ml streptomycin in a humidified incubator with 5% CO2 at 37° C. The human HCT116 cell line was obtained from the ATCC. The MLH1⁺ cell line was generated by correcting one chromosomal copy of the MLH1 gene using rAAV-mediated knock-in gene targeting.

Vectors:

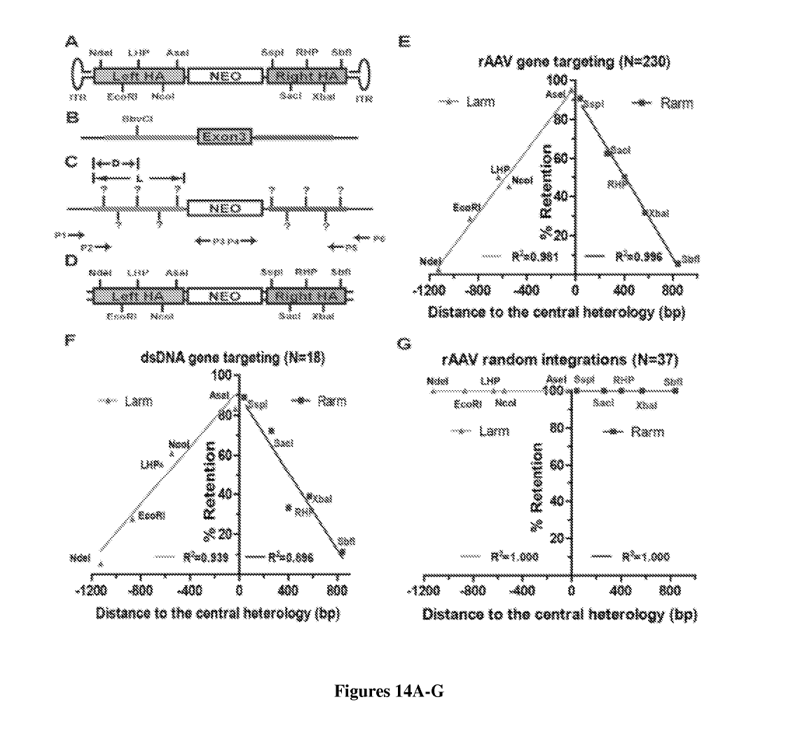

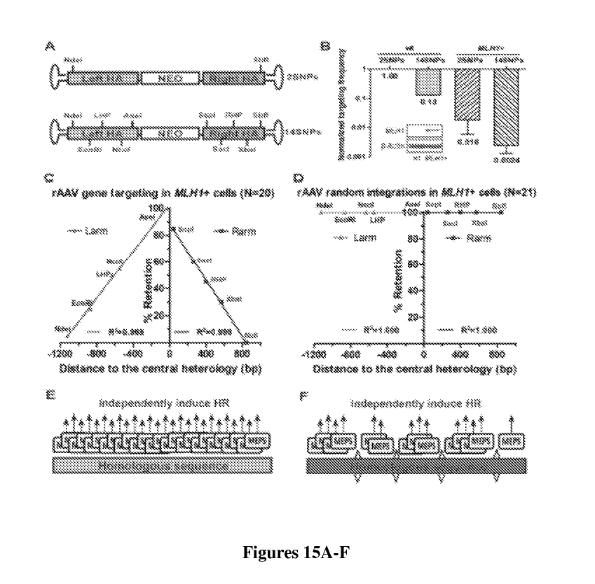

The HPRT targeting vectors (FIG. 14) were constructed using the rAAV system as described (Kohli et al. 2004). Briefly, the left and right homology arms were amplified by PCR from HCT116 genomic DNA. Viral single nucleotide polymorphisms (SNPs) and hairpin sequences were introduced by Quick-Change™ site-directed mutagenesis according to the manufacturer's (Agilent) instructions. The homology arms were attached to the drug selection cassette using fusion PCR before the product was ligated to the pAAV backbone. All virus packaging and infections were performed as described (Kohli et al. 2004).

Vector-Borne Marker Analysis:

Genomic DNA was Isolated using a PUREGENE DNA purification kit (Gentra Systems). The homology arms of the correctly targeted clones were amplified by diagnostic PCRs using primers illustrated in FIG. 14C. The retention of the vector-bore markers was analyzed by restriction digests (except for the hairpin on the right homology arm) and confirmed by DNA sequencing.

Targeting Efficiency Assay:

The targeting efficiency assay was modified from previous publications (Russell and Hirata 1998 and 2008). Briefly, 1×10⁶ cells were plated in 6-well plates on day 1. On day 2, the medium was changed and 100 ul of designated viral stock was added to the wells. On day 4, the cells were treated with trypsin, counted and aliquoted into 10 cm dishes for drug selection. The plates were fed either with 1 mg/ml G418 or 0.5 mg/ml G418+5 ug/ml 6-TG for 12 days, to identify totalclones and for those correctly gene targeted, respectively. The doubly drug-resistant colonies were confirmed to be correctly targeted by PCR using the primers illustrated in FIG. 14D. Results were averaged from 7 plates.

Results:

The hypoxanthine phosphoribosyltransferase (HPRT) locus on the X chromosome has been widely used as a negative selection marker (Russell and Hirata 2008; Rhomas and Capecchi 1986). Inactivation of HPRT by a single round of targeting confers 6-thioguanine (6-TG) resistance in hypoxanthine, aminopterin, and thymidine (HAT) pre-selected male cells. In this system, an rAAV targeting vector (FIG. 14A) has been assembled to disrupt exon 3 of HPRT (FIG. 4B) by replacing it with a NEO selection cassette. Following G418 selection, targeted and random events can be distinguished based on 6-TG resistance and sensitivity, respectively. In order to distinguish the viral sequence from the chromosomal counterpart, each homology arm (HA) of the virus was altered with 4 single nucleotide polymorphisms (SNPs) that generate unique restriction enzyme recognition sites. In addition, a hairpin structure composed of 3 clustered SNPs was also introduced into each HA. The hairpins were introduced because they are known to be refractory to MMR activity (de Massy 2003; FIG. 14A). The HAs of the targeted and random clones can be amplified from the integrated loci (FIG. 14C) using diagnostic PCRs. Primers P1:P3 and P4:P6 (targeting primers) specifically amplify the left and right HAs of targeted clones, whereas P2:P3 and P4:P5 (RI primers) amplify random clones with intact HAs (FIG. 14C). The retention of the viral SNPs and hairpins can then be analyzed by restriction length polymorphism analysis and sequencing, respectively.

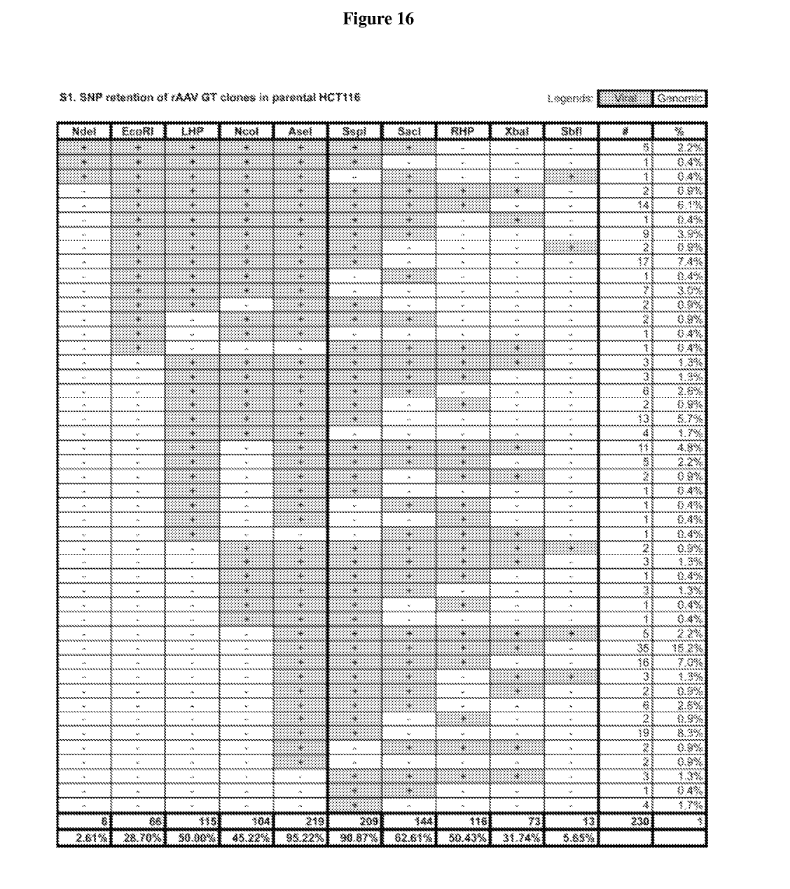

In order to illustrate the molecular mechanism of rAAV gene targeting, which part(s) of the HAs integrated into the genome was characterized. The initial gene targeting experiments were performed in the MMR-deficient HCT116 cell line. After rAAV infection, cells were selected with G418 and 6-TG for targeted clones. Around 60% of the G418R 6-TGR clones could be amplified by both targeting primer pairs, consistent with targeted integration. The other 40% of the clones did not yield PCR products using either primer pair and presumably resulted from spontaneous HPRT mutations (data not shown). A total of 230 targeted clones (all confirmed by PCR) were analyzed for the retention frequency of viral SNPs, which was plotted against the position of the SNPs on the Has (FIG. 14E and FIG. 16). Interestingly, the viral SNPs were retained in a gradient pattern. The inner SNPs had the highest chance of retention (219/230 for AseI and 209/230 for SspI), whereas the outer markers were frequently lost during GT (6/230 for NdeI and 13/230 for SbfI).

The linear SNP retention curve demonstrates that crossovers are evenly distributed throughout the HAs. When a crossover occurs during gene targeting, the HA to the outside of the crossover will be recombined out. The frequency a certain SNP being retained equals to the chance of the crossover happening to the outside of the SNP, assuming that a single crossover occurs on each strand of the HA. Accordingly, the frequency of crossovers occurring can be reversely calculated as the slope of the SNP retention curve, which for the data is the same at any point along the HA. This linear retention curve is in direct contrast to the exponential SNP retention reported in yeasts, flies and mouse embryonic stem cells (de Massy 2003; Hilliker et al. 1994; Stark et al. 2004; Elliot et al. 1998), which indicates that the mechanism of gene targeting in human somatic cells is different from lower organisms.

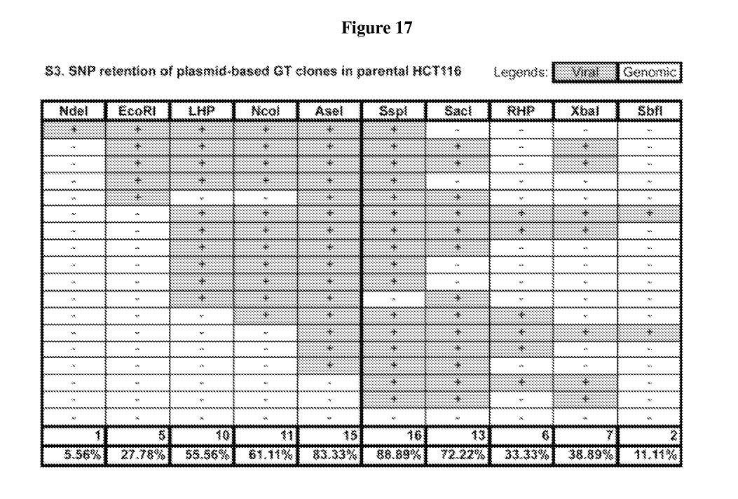

To determine if the linear SNP retention curve is intrinsic to the rAAV vector or is a general feature of GT in human somatic cells, a parallel experiment was performed using a plasmid-based vector that was identical to rAAV except that it is double-stranded and it did not contain the ITRs (FIG. 14D). Eighteen correctly targeted clones were recovered despite the extremely low targeting efficiency imparted by the dsDNA transfection methodology. SNP analysis, however, revealed an indistinguishable linear retention curve (FIG. 14F and FIG. 17). As a consequence, it is concluded that the gradient loss of the outer HAs is characteristic of gene targeting in human somatic cells.

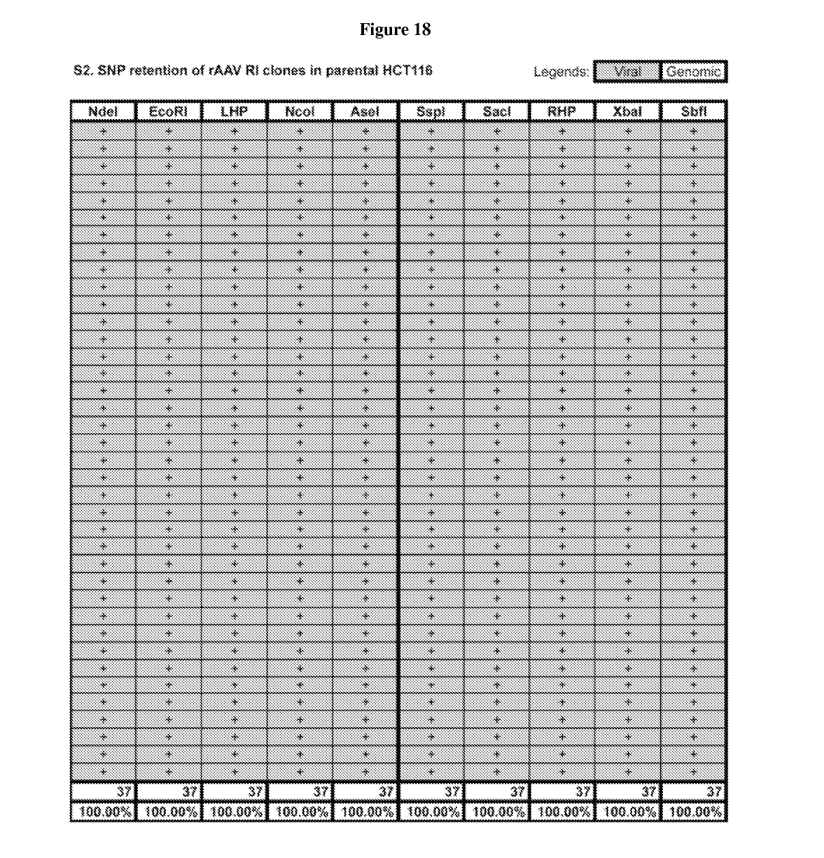

While gene targeting requires extended homology, random integrations are generally believed to be mediated by the non-homologous end joining (NHEJ) pathways. In order to test whether targeted and random integrations produce different molecular products, 38 G418R6-TGS clones were also recovered and analyzed by diagnostic PCRs. Thirty-seven of these random clones could be amplified by both sets of random integration primers, indicating that the entire HAs were integrated during random integration (data not shown). To rule out potential discontinuous HAs, SNP retention analysis was also performed upon the random clones. All the SNPs were retained at 100% frequency on both arms of the random clones (FIG. 14G and FIG. 18), which further confirms that the virus is integrated intact during random integrations. The result is consistent with previous observations that the viral-chromosomal DNA junctions almost exclusively reside on the ITRs instead of the HAs during random integrations (Miller et al. 2005; Nakai et al., 2001). In contrast to the gradient SNP retention during correct gene targeting, the retention of intact viral HAs during random events clearly demonstrates that rAAV GT and RIs are mediated by non-overlapping pathways.

SNPs generate mismatches in the hDNA intermediate, which are sensitive to the MMR system. To address the effect of mismatches on GT, another rAAV targeting vector was constructed with only 2 SNPs and tested in parental HCT116 cells (FIG. 15A). Targeting efficiency increased about 7.5-fold compared to the original vector, which contained a total of 14 SNPs (FIG. 15B), which indicates that mismatches disturb gene targeting even in a MMR-deficient background. To further address the role of the MMR system, gene targeting was performed in an MMR proficient variant (MLH1⁺), in which one copy of the MLH1 gene was corrected by a rAAV-mediated knock-in (Horizon Discovery). Western blot analysis demonstrated the restoration of MLH1 protein in these cell lines (FIG. 15B inset). Targeting efficiency decreased by more than 50-fold for each vector in MLH1⁺ cells (FIG. 15B) indicating a strong anti-recombination role of the MMR system (Siehler et al. 2009; Stone et al. 2008).

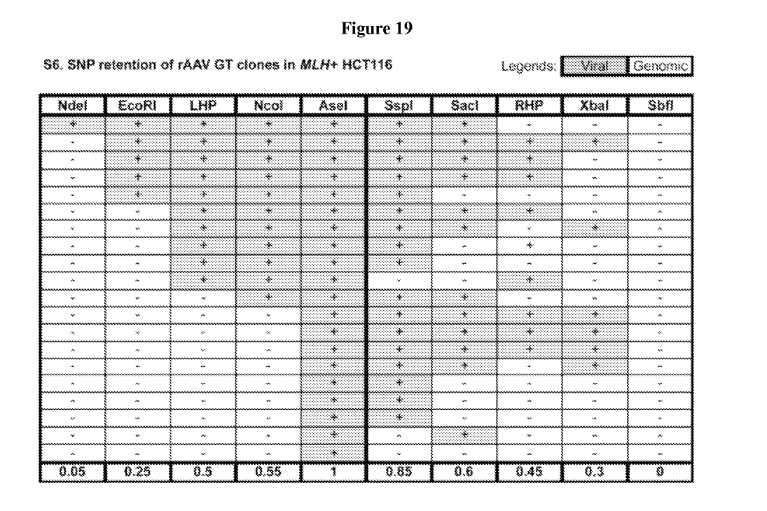

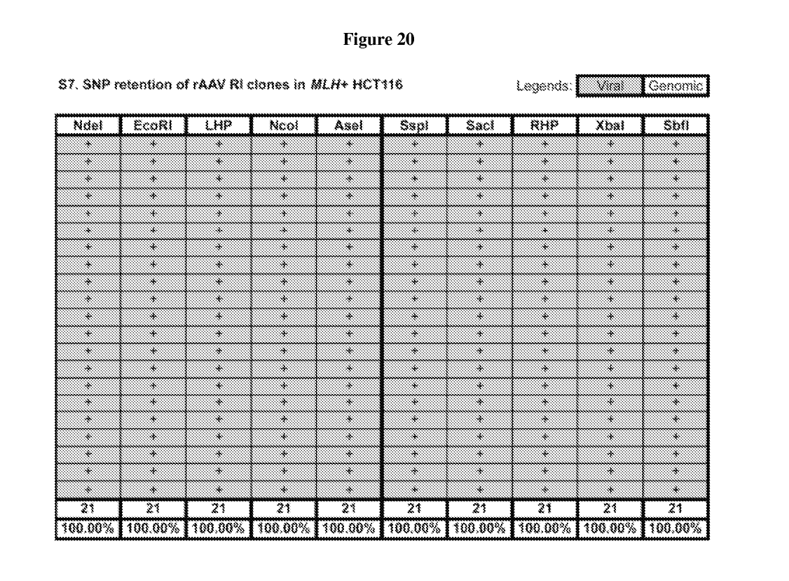

Since a strong anti-recombination effect of the MMR system was observed, it was next determined whether it could efficiently correct mismatches in the hDNA intermediate. Despite the extremely low targeting efficiency in MLH1⁺ cells, twenty correctly targeted clones were recovered and analyzed for SNP retention (FIG. 15C and FIG. 19). Surprisingly, the SNP retention curve in MLH1⁺ cells was not significantly different from the parental HCT116 (MLH1⁻) cell line. The hairpins, which are refractory to MMR5, were retained at the same frequency as predicted by the linear regression of neighboring SNPs. Moreover, the percentage of discontinuous HAs does not change significantly in the MMR-proficient background (FIG. 19). These results indicate that the MMR system exercises no detectable “spell-checker” activity upon mismatches in the gene targeting hDNA intermediate, consistent with the separation of functions between the “spell-checker” and the “anti-recombination” activities of the MMR system (Siehler et al. 2009; Stone et al. 2008). To test whether the MMR system affects random integration, 22 G418R6-TGS clones were also recovered from MLH1⁺ background and analyzed for SNP retention. Twenty-one of them could be amplified using the RI primers (data not shown), with all viral SNPs retained in the products (FIG. 15D and FIG. 20), which is consistent with the previous observation that the MMR system does not affect NHEJ (Siehler et al. 2009).

Discussion

Although rAAV has been widely used in laboratorial and clinical studies, the mechanism of rAAV-mediated GT remains obscure. Here, the impact of mismatches and MMR on rAAV-mediated gene targeting was investigated. Mismatches reduce the efficiency of homologous recombination in an MMR repair-independent mechanism. Thus, the MMR system maintains genomic stability not only by correcting mismatches in hDNA, but also by inhibiting recombination of homeologous (non-identical) sequences (Nicholson et al. 2000). Disruption of the MMR system is associated with increased HR activity in mammalian cells (Ciotta et al. 1998; de Wind et al. 1995), although the effect of the number of mismatches on this process is not fully characterized in human cells. With the high-efficiency rAAV GT system, targeting efficiency of homeologous sequences in a MMR-proficient background were compared. It was discovered that gene targeting efficiency decreased dramatically in a MMR-proficient background, which was consistent with the observations that a single mismatch is sufficient to inhibit HR in yeast (Datta et al. 1997; Chen and Jinks-Robertson, 1999). Interestingly, it was also observed that increasing the number of mismatches decreased targeting efficiency even in the MMR-deficient background.

The findings indicate: (1) the initial sites of crossovers are evenly distributed along the HAs, and (2) mismatches greatly reduce targeting efficiency independent of the repair activity of the MMR system. These results can be uniformly explained by the minimal efficient processing segment (MEPS) theory (Shen and Huang 1986). MEPS are defined as the minimal length of homology, below which recombination becomes inefficient (Shen and Huang 1986; Datta et al. 1997). MEPS serve as a basic unit of HR, which can initiate crossovers independently with the same efficiency. The recombinogenicity of a certain HA can be directly assessed as the number of overlapping MEPS in it. For example, an L by long uninterrupted homology is composed of (L−M+1) MEPS, where M is the length of MEPS, and its tendency to induce HR can be measured as:

F=E(L−M+1)≈E(L−M)

where E is the recombination efficiency of a single MEPS (FIG. 15E). Because MEPS are evenly distributed throughout the HAs (except near mismatches or the HA ends), they can initiate crossovers with equal frequency, which likely shaped the linear SNP retention curve observed (FIGS. 14E, 14F and 15C).

Mismatches reduce the number of MEPS by disrupting homology. For example, when X mismatches are introduced into a HA with a length of L bp, the number of MEPS equals the sum of MEPS in each homologous segment, which can be as low as (L−XM) depending on the positions of the mismatches (FIG. 15F):

F=EΣ(Li−M), (0<i≦X)

The otherwise paradoxical observation that the effect of mismatches is independent of the MMR repair system is due to the fact that the decreased number of MEPS is related to the number of mismatches, but, independent of the MMR repair activity.

As an extrapolation of the MEPS theory, the targeting efficiency of a targeting vector equals to the chance of crossovers occurring independently on both HAs:

F=FL*FR

where FL and FR represent the length of the left and right HAs, respectively. If the length of one HA is kept constant and the other HA is reduced, the targeting efficiency will decrease linearly.

The minimal length of a rAAV HA is approximately 150 bp (Hirata and Russell 2000). As a proof of principle, if one plugs in M=150 into the previous equation and calculates the targeting efficiency of the 2 and 14 SNP-containing vectors according to the positions of the mismatches (FIG. 15A), the targeting efficiency is expected to decreased by 8.3-fold when comparing targeting vectors with 2 or 14 SNPs, which is very close to the experimental determined value 7.5-fold (FIG. 15B). Thus, it is concluded that the MEPs theory is applicable to rAAV-mediated gene targeting in human somatic cells.

### BIBLIOGRAPHY

1. Birmingham, E. C., et al. 2004. Genetics 168:1539-55.
2. Chen, F., et al. 2011. Nature methods 8:753-5.
3. Datta, A., et al. 1996. Molecular and cellular biology 16:1085-93.
4. Dekker, M., et al. 2006. Gene therapy 13:686-94.
5. Elliott, B., and M. Jasin. 2001. Molecular and cellular biology 21:2671-82.
6. Evans, E., and E. Alani. 2000. Molecular and cellular biology 20:7839-44.
7. Fattah, F., et al. 2010. PLoS Genet 6:e1000855.
8. Fishman-Lobell, J., et al. 1992. Molecular and cellular biology 12:1292-303.
9. Friedberg, E. C., et al. 1995. Nature medicine 17:759.
11. Gustin, J. P., et al. 2009. PNAS United States of America 106:2835-40.
12. Hastings, P. J., et al. 1993. Genetics 135:973-80.
13. Heyer, W. D., et al. 2006. Nucleic acids research 34:4115-25.
14. Hirata, R., J. et al. 2002. Nature biotechnology 20:735-8.
15. Hiyama, T., et al. 2006. Nucleic acids research 34:880-92.
16. Iftode, C., Y et. al.. 1999. Critical reviews in biochemistry and molecular biology 34:141-80.
17. Igoucheva, O., et al. 2004. Current molecular medicine 4:445-63.
18. Inagaki, K., et al. 2007. J Virol 81:11290-303.
19. Jasin, M., et al. 1990. Genes & development 4:157-66.
20. Kawabata, M., et al. 2005. Acta medica Okayama 59:1-9.
21. Khan, I. F., et al. 2011. Nat Protoc 6:482-501.
22. Langston, L. D., and L. S. Symington. 2004. PNAS USA 101:15392-7.
23. Langston, L. D., and L. S. Symington. 2005. The EMBO journal 24:2214-23.
24. Leung, W., et al. 1997. PNAS USA 94:6851-6.
25. Li, J., et al. 2001. Molecular and cellular biology 21:501-10.
26. Lieber, M. R. 2008. The Journal of biological chemistry 283:1-5.
27. Lu, I. L., et al. 2003. Gene therapy 10:1910-6.
28. Masramon, L., et al. 2000. Cancer Genet Cytogenet 121:17-21.
29. Miller, D. G, et al. 2005. J Virol 79:11434-42.
30. Miyagawa, K., et al. 2002. The EMBO journal 21:175-80.
31. Moerschell, R. P., et al. 1988. PNAS USA 85:524-8.
32. Negritto, M. T., et al. 1997. Molecular and cellular biology 17:278-86.
33. Passy, S. I., et al. 1999. PNAS USA 96:4279-84.
34. Pierce, E. A., et al. 2003. Gene therapy 10:24-33.
35. Porteus, M. H., and D. Baltimore. 2003. Science 300:763.
36. Porteus, M. H., et al. 2003. Molecular and cellular biology 23:3558-65.
37. Preston, B. D., et al. 2010. Seminars in cancer biology 20:281-93.
38. Radecke, S., et al. 2006. The journal of gene medicine 8:217-28.
39. Rago, C., et al. 2007. Nature protocols 2:2734-46.
40. Ruis, B. L., et al. 2008. Mol Cell Biol 28:6182-95.
41. Russell, D. W., and R. K. Hirata. 1998. Nature genetics 18:325-30.
42. Schwartz, R. A., et al. 2007. Journal of virology 81:12936-45.
43. Sharma, S., et al. 2006. The Biochemical journal 398:319-37.
44. Shirasawa, S., et al. 1993. Science 260:85-8.
45. Smithies, O., et al. 1985. Nature 317:230-4.
46. Solinger, J. A., et al. 2002. Molecular cell 10:1175-88.
47. Song, K. Y, et al. 1987. PNAS USA 84:6820-4.
48. Sugiyama, T., and S. C. Kowalczykowski. 2002. The Journal of biological chemistry 277:31663-72.
49. Sung, P., et al. 2000. Mutation research 451:257-75.
50. Symington, L. S., and J. Gautier. 2010. Annual review of genetics.
51. Szostak, J. W., et al. 1983. Cell 33:25-35.
52. Thacker, J. 2005. Cancer letters 219:125-35.
53. Thacker, J., et al. 1994. Mutagenesis 9:163-8.
54. Topaloglu, O., P et. al.. 2005. Nucleic acids research 33:e158.
55. Trobridge, G, et al. 2005. Human gene therapy 16:522-6.
56. Umar, A., et al. 1994. Science 266:814-6.
57. Waldman, T., et al. 1995. Cancer research 55:5187-90.
58. Wechsler, T., et al. 2011. Nature 471:642-6.
59. Wood, A. J., et al. 2011. Science 333:307.
60. Yoshihara, T., et al. 2004. The EMBO journal 23:670-80.
61. Zheng, H., et al. 1991. PNAS USA 88:8067-71.
Carter B J (2004) Mol Ther 10:981-989.
Cattell, E., et al. 2010. Environ Mol Mutagen 51:635-45.
Chen, I (2008) Nature Struct. Mol. Biol. 15:699.
Fattah K R, et al. (2008) DNA Repair 7:762-774.
Fattah F, et al. (2008) Proc. Natl. Acad. Sci., USA 105:8703-8708.
Fattah F, et al. (2010) PLoS Genetics, 6:e1000855.
Hastings P J, et al. (1993) Genetics 135:973-980.
Hendrickson E A, et al. (2006) in DNA Damage Recognition, Structural aspects of Ku and the DNA-dependent protein kinase complex, eds. Seide W, Kow Y W, Doetsch P (Taylor and Francis, New York), pp 629-684.
Hendrickson E A (2008) in Sourcebook of Models for Biomedical Research, Gene targeting in human somatic cells, ed. Conn P M (Humana, Totowa, N.J.), pp 509-525.
Heyer W D, et al. (2006) Nucleic Acids Res 34:4115-4125.
Inagaki K, et al. (2007) J Virol 81:11290-11303.
Inagaki, K., et al. 2007. J Virol 81:11304-21.
Khan, I. et al. 2011. Nat Protoc 6:482-501.
Kohli, M., et al. 2004. Nucleic Acids Res 32:e3.
Kurosawa, A., and N. Adachi. 2010. J Radiat Res (Tokyo) 51:503-9.
Li G, Nelsen C, Hendrickson E A (2002) Proc Natl Acad Sci USA 99:832-837.
Ma, Y, et al. 2002. Cell 108:781-94.
Masramon, L., et al. 2000. Cancer Genet Cytogenet 121:17-21.
Miller, et al. 2005. J Virol 79:11434-42.
Moshous, D., et al. 2001. Cell 105:177-86.
Nicolas, N., et al. 1998. J Exp Med 188:627-34.
Rooney, S., et al. 2002. Mol Cell 10:1379-90.
Ruis B, et al. (2008) Mol. Cell. Biol. 28:6182-6195.
Russell D W, Hirata R K (1998) Nat Genet 18:325-330.
Spagnolo L, et al. (2006) Mol Cell 22:511-519.
Thomas K R, Capecchi M R (1987) Cell 51:503-512.
van Veelen L, Wesoly J, Kanaar R (2006) in DNA Damage Recognition, Biochemical and cellular aspects of homologous recombination, eds Seide W, Kow Y W, Doetsch P (Taylor and Francis, New York), pp 581-607.
Wang Y, et al. (2009) Proc. Natl. Acad. Sci., USA, 106:1243-12435.
Yan, Y., et al. 2010. Future Oncol 6:1015-29.
Kohli M, et al. Nucleic Acids Res 2004; 32:e3.
Russell D W, Hirata R K. Nat Genet 1998; 18:325-330.
Russell D W, Hirata R K. Hum Gene Ther 2008; 19:907-914.
Thomas K R, Capecchi M R. Nature 1986; 324:34-38.
McCulloch R D, Baker M D. Genetics 2006; 172:1767-1781.
de Massy B. Trends Genet 2003; 19:514-522.
Hilliker A J, et al. Genetics 1994; 137:1019-1026.
Stark J M, et al Mol Cell Biol 2004; 24:9305-9316.
Elliott B, et al. Mol Cell Biol 1998;18:93-101.
Siehler S Y, et al. DNA Repair (Amst) 2009; 8:242-252.
Stone J E, et al. Genetics 2008; 178:1221-1236.
Nicholson A, et al. Genetics 2000; 154:133-146.
Ciotta C, et al. J Mol Biol 1998; 276:705-719.
de Wind N, et al. Cell 1995; 82:321-330.
Datta A, Hendrix M, Proc Natl Acad Sci USA 1997; 94:9757-9762.
Chen W, Jinks-Robertson S. Genetics 1999; 151:1299-1313.
Miller D G, et al. J Virol 2005; 79:11434-11442.
Nakai H, et al. J Virol 2001; 75:6969-6976.
Shen P, Huang Genetics 1986; 112:441-457.
Hirata R K, Russell D W. J Virol 2000; 74:4612-4620.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method to increase gene targeting frequency comprising inhibiting expression of at least one gene of a mismatch repair pathway or by inhibiting activity of at least one protein of a mismatch repair pathway so as to provide increased gene targeting frequency as compared to a cell in which expression and/or activity has not be inhibited.

2. A method to increase gene targeting frequency comprising increasing expression of at least one gene coding for Rad52, Rad57, Rad59, MUS81, XRCC3 or a combination thereof so as to provide increased gene targeting frequency as compared to a cell in which expression has not been increased.

3. The method of claim 1, wherein the gene or protein is MLH1, PMS2, MSH2, MSH6, MSH3, PMS1, MLH3 or a combination thereof.

4. The method of claim 1, wherein the gene or protein is MLH1.

5. The method of claim 1, wherein the gene or protein is MSH2.

6. The method of claim 1, wherein expression is transiently inhibited.

7. The method of claim 1, wherein the protein activity is inhibited by a small molecule or expression of the protein is inhibited by antisense, siRNA or shRNA.

8. The method of claim 1, wherein the DNA assimilation and/or targeting is mediated by a retrovirus, rAAV, dsDNA, ssDNA, zinc finger nuclease, homing nuclease, meganuclease, transcription activator like (TAL) effector nuclease or a combination thereof.

9. The method of claim 1, wherein the DNA assimilation and/or targeting is mediated by rAAV.

10. The method of claim 1, wherein the cell in which the mismatch repair gene or protein expression/activity is to be inhibited is mismatch repair proficient.


SEQUENCE LISTING (64 sequences)

SEQ ID NO: 1 - DNA, 53 bp, Artificial Sequence
  A synthetic oligonucleotide
atacatacgcggccgctcaagcactggctatgcatgtataccatatgcaaaag

SEQ ID NO: 2 - DNA, 64 bp, Artificial Sequence
  A synthetic oligonucleotide
ttatccgcggtggagctccagcttttgttccctttagtcaggaatttaatagaaagtttcatac

SEQ ID NO: 3 - DNA, 65 bp, Artificial Sequence
  A synthetic oligonucleotide
ttatggtacccaattcgccctatagtgagtcgtattacttgctttcatttcacttggttacagtg

SEQ ID NO: 4 - DNA, 50 bp, Artificial Sequence
  A synthetic oligonucleotide
atacatacgcggccgcttaaatggctgcccaatcacctgcaggattgatg

SEQ ID NO: 5 - DNA, 26 bp, Artificial Sequence
  A synthetic oligonucleotide
ttgaatgcttgcattgtatgtctggc

SEQ ID NO: 6 - DNA, 24 bp, Artificial Sequence
  A synthetic oligonucleotide
aaagcgcctcccctacccggtagg

SEQ ID NO: 7 - DNA, 20 bp, Artificial Sequence
  A synthetic oligonucleotide
acgtgaccctgttcatcagc

SEQ ID NO: 8 - DNA, 26 bp, Artificial Sequence
  A synthetic oligonucleotide
aaacaagtctttaattcaagcaagac

SEQ ID NO: 9 - DNA, 24 bp, Artificial Sequence
  A synthetic oligonucleotide
ccacgtaacacatcctttgccctc

SEQ ID NO: 10 - DNA, 38 bp, Artificial Sequence
  A synthetic oligonucleotide
atacatacgcggccgcgagcagtacctagtacgttgac

SEQ ID NO: 11 - DNA, 31 bp, Artificial Sequence
  A synthetic oligonucleotide
ggactagtcatgcggctacttatgtattctg

SEQ ID NO: 12 - DNA, 28 bp, Artificial Sequence
  A synthetic oligonucleotide
ccagctcgagggccagaaggtaggagaa

SEQ ID NO: 13 - DNA, 34 bp, Artificial Sequence
  A synthetic oligonucleotide
atacatacgcggccgcggctgagacacaactctg

SEQ ID NO: 14 - DNA, 60 bp, Artificial Sequence
  A synthetic oligonucleotide
gactagtataacttcgtatagcatacattatacgaagttatgcatggctgaacaagatgg

SEQ ID NO: 15 - DNA, 31 bp, Artificial Sequence
  A synthetic oligonucleotide
ccagctcgagcatacatatgcacagtggtac

SEQ ID NO: 16 - DNA, 20 bp, Artificial Sequence
  A synthetic oligonucleotide
cactgctatgatgcctaatg

SEQ ID NO: 17 - DNA, 22 bp, Artificial Sequence
  A synthetic oligonucleotide
gagcagtacctagtacgttgac

SEQ ID NO: 18 - DNA, 19 bp, Artificial Sequence
  A synthetic oligonucleotide
aggtgagatgacaggagat

SEQ ID NO: 19 - DNA, 53 bp, Artificial Sequence; identical to SEQ ID NO: 1
  A synthetic oligonucleotide

SEQ ID NO: 20 - DNA, 38 bp, Artificial Sequence
  A synthetic oligonucleotide
gttcagctttattcaagtggaattcctgggtcaagggg

SEQ ID NO: 21 - DNA, 38 bp, Artificial Sequence
  A synthetic oligonucleotide
ccccttgacccaggaattccacttgaataaagctgaac

SEQ ID NO: 22 - DNA, 47 bp, Artificial Sequence
  A synthetic oligonucleotide
ttcaagcgatccttccaccccagctacgtgagtagctgggactatag

SEQ ID NO: 23 - DNA, 47 bp, Artificial Sequence
  A synthetic oligonucleotide
ctatagtcccagctactcacgtagctggggtggaaggatcgcttgaa

SEQ ID NO: 24 - DNA, 27 bp, Artificial Sequence
  A synthetic oligonucleotide
agggttcgccatggtacccagcctccc

SEQ ID NO: 25 - DNA, 27 bp, Artificial Sequence
  A synthetic oligonucleotide
gggaggctgggtaccatggcgaaccct

SEQ ID NO: 26 - DNA, 59 bp, Artificial Sequence
  A synthetic oligonucleotide
ttaatgactaagaggtgtttgttataaagattaatgtatgaaactttctattaaattcc

SEQ ID NO: 27 - DNA, 59 bp, Artificial Sequence
  A synthetic oligonucleotide
ggaatttaatagaaagtttcatacattaatctttataacaaacacctcttagtcattaa

SEQ ID NO: 28 - DNA, 53 bp, Artificial Sequence
  A synthetic oligonucleotide
acttggttacagtgagatttttctaaaatattcactagtactttacatcaaag

SEQ ID NO: 29 - DNA, 53 bp, Artificial Sequence
  A synthetic oligonucleotide
ctttgatgtaaagtactagtgaatattttagaaaaatctcactgtaaccaagt

SEQ ID NO: 30 - DNA, 55 bp, Artificial Sequence
  A synthetic oligonucleotide
tttttattactttagactattgtttctagattttatgttgggttggtatttcctg

SEQ ID NO: 31 - DNA, 55 bp, Artificial Sequence
  A synthetic oligonucleotide
caggaaataccaacccaacataaaatctagaaacaatagtctaaagtaataaaaa

SEQ ID NO: 32 - DNA, 46 bp, Artificial Sequence
  A synthetic oligonucleotide
ctgcaggtatactcaagagagtataccgcaccagaaaccacttttg

SEQ ID NO: 33 - DNA, 46 bp, Artificial Sequence
  A synthetic oligonucleotide
caaaagtggtttctggtgcggtatactctcttgagtatacctgcag

SEQ ID NO: 34 - DNA, 41 bp, Artificial Sequence
  A synthetic oligonucleotide
gagtcaaaagtcctttggagctcggtttgacaaataaggtg

SEQ ID NO: 35 - DNA, 41 bp, Artificial Sequence
  A synthetic oligonucleotide
caccttatttgtcaaaccgagctccaaaggacttttgactc

SEQ ID NO: 36 - DNA, 50 bp, Artificial Sequence; identical to SEQ ID NO: 4
  A synthetic oligonucleotide

SEQ ID NO: 37 - DNA, 41 bp, Artificial Sequence
  A synthetic oligonucleotide
atacatacgcggccgcgagccaccatgtccaactggtttag

SEQ ID NO: 38 - DNA, 62 bp, Artificial Sequence
  A synthetic oligonucleotide
ttatccgcggtggagctccagcttttgttccctttagaaaagaacaaaaactcatgaatatg

SEQ ID NO: 39 - DNA, 60 bp, Artificial Sequence
  A synthetic oligonucleotide
atggtacccaattcgccctatagtgagtcgtattactattttgctacttgtgtttttaag

SEQ ID NO: 40 - DNA, 58 bp, Artificial Sequence
  A synthetic oligonucleotide
atacatacgcggccgcgtcaataagtaaatacaaataaagtaataaaaaattattggc

SEQ ID NO: 41 - DNA, 23 bp, Artificial Sequence
  A synthetic oligonucleotide
cgccctatagtgagtcgtattac

SEQ ID NO: 42 - DNA, 58 bp, Artificial Sequence; identical to SEQ ID NO: 40
  A synthetic oligonucleotide

SEQ ID NO: 43 - DNA, 27 bp, Artificial Sequence
  A synthetic oligonucleotide
gtcacaggtgaccaaaaaaaattactg

SEQ ID NO: 44 - DNA, 32 bp, Artificial Sequence
  A synthetic oligonucleotide
ttcttgacgagttcttctgaggggatcaattc

SEQ ID NO: 45 - DNA, 25 bp, Artificial Sequence
  A synthetic oligonucleotide
gagccaccatgtccaactggtttag

SEQ ID NO: 46 - DNA, 24 bp, Artificial Sequence
  A synthetic oligonucleotide
aaagcgcctcccctacccggtagg

SEQ ID NO: 47 - DNA, 26 bp, Artificial Sequence
  A synthetic oligonucleotide
actgggtctaatgatggccacacgac

SEQ ID NO: 48 - DNA, 24 bp, Artificial Sequence
  A synthetic oligonucleotide
cccttgggctaaggaatcctctgg

SEQ ID NO: 49 - DNA, 26 bp, Artificial Sequence
  A synthetic oligonucleotide
aatgtttgcttaaaaacacaagtagc

SEQ ID NO: 50 - DNA, 26 bp, Artificial Sequence
  A synthetic oligonucleotide
gccaaataacactagatgttaggaac

SEQ ID NO: 51 - DNA, 40 bp, Artificial Sequence
  A synthetic oligonucleotide
atacatacgcggccgcgtctatacagagcaatcacaatgg

SEQ ID NO: 52 - DNA, 20 bp, Artificial Sequence
  A synthetic oligonucleotide
agcatctcagggccaaaaat

SEQ ID NO: 53 - DNA, 18 bp, Artificial Sequence
  A synthetic oligonucleotide
tgggacccactccatcga

SEQ ID NO: 54 - DNA, 17 bp, Artificial Sequence
  A synthetic oligonucleotide
ctagctacagtgaaatc

SEQ ID NO: 55 - DNA, 16 bp, Artificial Sequence
  A synthetic oligonucleotide
tagctacagagaaatc

SEQ ID NO: 56 - DNA, 2661 bp, Homo sapiens
gaagagacccagcaacccacagagttgagaaatttgactggcattcaagctgtccaatcaatagctgccgctgaagggtggggctggatggcgtaagctacagctgaaggaagaacgtgagcacgaggcactgaggtgattggctgaaggcacttccgttgagcatctagacgtttccttggctcttctggcgccaaaatgtcgttcgtggcaggggttattcggcggctggacgagacagtggtgaaccgcatcgcggcgggggaagttatccagcggccagctaatgctatcaaagagatgattgagaactgtttagatgcaaaatccacaagtattcaagtgattgttaaagagggaggcctgaagttgattcagatccaaacaatggcaccgggatcaggaaagaagatctggatattgtatgtgaaaggttcactactagtaaactgcagtcctttgaggatttagccagtatttctacctatggctttcgaggtgaggctttggccagcataagccatgtggctcatgttactattacaacgaaaacagctgatggaaagtgtgcatacagagcaagttactcagatggaaaactgaaagcccctcctaaaccatgtgctggcaatcaagggacccagatcacggtggaggaccttttttacaacatagccacgaggagaaaagctttaaaaaatccaagtgaagaatatgggaaaattttggaagttgttggcaggtattcagtacacaatgcaggcattagtttctcagttaaaaaacaaggagagacagtagctgatgttaggacactacccaatgcctcaaccgtggacaatattcgctccatctttggaaatgctgttagtcgagaactgatagaaattggatgtgaggataaaaccctagccttcaaaatgaatggttacatatccaatgcaaactactcagtgaagaagtgcatcttcttactcttcatcaaccatcgtctggtagaatcaacttccttgagaaaagccatagaaacagtgtatgcagcctatttgcccaaaaacacacacccattcctgtacctcagtttagaaatcagtccccagaatgtggatgttaatgtgcaccccacaaagcatgaagttcacttcctgcacgaggagagcatcctggagcgggtgcagcagcacatcgagagcaagctcctgggctccaattcctccaggatgtacttcacccagactttgctaccaggacttgctggcccctctggggagatggttaaatccacaacaagtctgacctcgtcttctacttctggaagtagtgataaggtctatgcccaccagatggttcgtacagattcccgggaacagaagcttgatgcatttctgcagcctctgagcaaacccctgtccagtcagccccaggccattgtcacagaggataagacagatatttctagtggcagggctaggcagcaagatgaggagatgcttgaactcccagcccctgctgaagtggctgccaaaaatcagagcttggagggggatacaacaaaggggacttcagaaatgtcagagaagagaggacctacttccagcaaccccagaaagagacatcgggaagattctgatgtggaaatggtggaagatgattcccgaaaggaaatgactgcagcttgtaccccccggagaaggatcattaacctcactagtgttttgagtctccaggaagaaattaatgagcagggacatgaggttctccgggagatgttgcataaccactccttcgtgggctgtgtgaatcctcagtgggccttggcacagcatcaaaccaagttataccttctcaacaccaccaagcttagtgaagaactgttctaccagatactcatttatgattttgccaattttggtgttctcaggttatcggagccagcaccgctctttgaccttgccatgcttgccttagatagtccagagagtggctggacagaggaagatggtcccaaagaaggacttgctgaatacattgttgagtttctgaagaagaaggctgagatgcttgcagactatttctctttggaaattgatgaggaagggaacctgattggattaccccttctgattgacaactatgtgccccctttggagggactgcctatcttcattcttcgactagccactgaggtgaattgggacgaagaaaaggaatgttttgaaagcctcagtaaagaatgcgctatgttctattccatccggaagcagtacatatctgaggagtcgaccctctcaggccagcagagtgaagtgcctggctccattccaaactcctggaagtggactgtggaacacattgtctataaagccttgcgctcacacattctgcctcctaaacatttcacagaagatggaaatatcctgcagcttgctaacctgcctgatctatacaaagtctttgagaggtgttaaatatggttatttatgcactgtgggatgtgttcttctttctctgtattccgatacaaagtgttgtatcaaagtgtgatatacaaagtgtaccaacataagtgttggtagcacttaagacttatacttgccttctgatagtattcctttatacacagtggattgattataaataaatagatgtgtcttaacataa

SEQ ID NO: 57 - DNA, 2851 bp, Homo sapiens
agccaatgggagttcaggaggcggagcgcctgtgggagccctggagggaactttcccagtccccgaggcggatcgggtgttgcatccatggagcgagctgagagctcgagtacagaacctgctaaggccatcaaacctattgatcggaagtcagtccatcagatttgctctgggcaggtggtactgagtctaagcactgcggtaaaggagttagtagaaaacagtctggatgctggtgccactaatattgatctaaagcttaaggactatggagtggatcttattgaagtttcagacaatggatgtggggtagaagaagaaaacttcgaaggcttaactctgaaacatcacacatctaagattcaagagtttgccgacctaactcaggttgaaacttttggctttcggggggaagctctgagctcactttgtgcactgagcgatgtcaccatttctacctgccacgcatcggcgaaggttggaactcgactgatgtttgatcacaatgggaaaattatccagaaaaccccctacccccgccccagagggaccacagtcagcgtgcagcagttattttccacactacctgtgcgccataaggaatttcaaaggaatattaagaaggagtatgccaaaatggtccaggtcttacatgcatactgtatcatttcagcaggcatccgtgtaagttgcaccaatcagcttggacaaggaaaacgacagcctgtggtatgcacaggtggaagccccagcataaaggaaaatatcggctctgtgtttgggcagaagcagttgcaaagcctcattccttttgttcagctgccccctagtgactccgtgtgtgaagagtacggtttgagctgttccgatgctctgcataatcttttttacatctcaggtttcatttcacaatgcacgcatggagttggaaggagttcaacagacagacagtttttctttatcaaccggcggccttgtgacccagcaaaggtctgcagactcgtgaatgaggtctaccacatgtataatcgacaccagtatccatttgttgttcttaacatttctgttgattcagaatgcgttgatatcaatgttactccagataaaaggcaaattttgctacaagaggaaaagcttttgttggcagttttaaagacctctttgataggaatgtttgatagtgatgtcaacaagctaaatgtcagtcagcagccactgctggatgttgaaggtaacttaataaaaatgcatgcagcggatttggaaaagcccatggtagaaaagcaggatcaatccccttcattaaggactggagaagaaaaaaaagacgtgtccatttccagactgcgagaggccttttctcttcgtcacacaacagagaacaagcctcacagcccaaagactccagaaccaagaaggagccctctaggacagaaaaggggtatgctgtcttctagcacttcaggtgccatctctgacaaaggcgtcctgagacctcagaaagaggcagtgagttccagtcacggacccagtgaccctacggacagagcggaggtggagaaggactcggggcacggcagcacttccgtggattctgaggggttcagcatcccagacacgggcagtcactgcagcagcgagtatgcggccagctccccaggggacaggggctcgcaggaacatgtggactctcaggagaaagcgcctgaaactgacgactctttttcagatgtggactgccattcaaaccaggaagataccggatgtaaatttcgagttttgcctcagccaactaatctcgcaaccccaaacacaaagcgttttaaaaaagaagaaattctttccagttctgacatttgtcaaaagttagtaaatactcaggacatgtcagcctctcaggttgatgtagctgtgaaaattaataagaaagttgtgcccctggacttttctatgagttctttagctaaacgaataaagcagttacatcatgaagcacagcaaagtgaaggggaacagaattacaggaagtttagggcaaagatttgtcctggagaaaatcaagcagccgaagatgaactaagaaaagagataagtaaaacgatgtttgcagaaatggaaatcattggtcagtttaacctgggatttataataaccaaactgaatgaggatatcttcatagtggaccagcatgccacggacgagaagtataacttcgagatgctgcagcagcacaccgtgctccaggggcagaggctcatagcacctcagactctcaacttaactgctgttaatgaagctgttctgatagaaaatctggaaatatttagaaagaatggctttgattttgttatcgatgaaaatgctccagtcactgaaagggctaaactgatttccttgccaactagtaaaaactggaccttcggaccccaggacgtcgatgaactgatcttcatgctgagcgacagccctggggtcatgtgccggccttcccgagtcaagcagatgtttgcctccagagcctgccggaagtcggtgatgattgggactgctcttaacacaagcgagatgaagaaactgatcacccacatgggggagatggaccacccctggaactgtccccatggaaggccaaccatgagacacatcgccaacctgggtgtcatttctcagaactgaccgtagtcactgtatggaataattggttttatcgcagatttttatgttttgaaagacagagtcttcactaaccttttttgttttaaaatgaacctgctacttaaaaaaaatacacatcacacccatttaaaagtgatcttgagaaccttttcaaaccagaaaaaaaaaaaaaaaa

SEQ ID NO: 58 - DNA, 3145 bp, Homo sapiens
ggcgggaaacagcttagtgggtgtggggtcgcgcattttcttcaaccaggaggtgaggaggtttcgacatggcggtgcagccgaaggagacgctgcagttggagagcgcggccgaggtcggcttcgtgcgcttctttcagggcatgccggagaagccgaccaccacagtgcgccttttcgaccggggcgacttctatacggcgcacggcgaggacgcgctgctggccgcccgggaggtgttcaagacccagggggtgatcaagtacatggggccggcaggagcaaagaatctgcagagtgttgtgcttagtaaaatgaattttgaatcttttgtaaaagatcttcttctggttcgtcagtatagagttgaagtttataagaatagagctggaaataaggcatccaaggagaatgattggtatttggcatataaggcttctcctggcaatctctctcagtttgaagacattctctttggtaacaatgatatgtcagcttccattggtgttgtgggtgttaaaatgtccgcagttgatggccagagacaggttggagttgggtatgtggattccatacagaggaaactaggactgtgtgaattccctgataatgatcagttctccaatcttgaggctctcctcatccagattggaccaaaggaatgtgttttacccggaggagagactgctggagacatggggaaactgagacagataattcaaagaggaggaattctgatcacagaaagaaaaaaagctgacttttccacaaaagacatttatcaggacctcaaccggttgttgaaaggcaaaaagggagagcagatgaatagtgctgtattgccagaaatggagaatcaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcagggttctgttgaagataccactggctctcagtctctggctgccttgctgaataagtgtaaaacccctcaaggacaaagacttgttaaccagtggattaagcagcctctcatggataagaacagaatagaggagagattgaatttagtggaagcttttgtagaagatgcagaattgaggcagactttacaagaagatttacttcgtcgattcccagatcttaaccgacttgccaagaagtttcaaagacaagcagcaaacttacaagattgttaccgactctatcagggtataaatcaactacctaatgttatacaggctctggaaaaacatgaaggaaaacaccagaaattattgttggcagtttttgtgactcctcttactgatcttcgttctgacttctccaagtttcaggaaatgatagaaacaactttagatatggatcaggtggaaaaccatgaattccttgtaaaaccttcatttgatcctaatctcagtgaattaagagaaataatgaatgacttggaaaagaagatgcagtcaacattaataagtgcagccagagatcttggcttggaccctggcaaacagattaaactggattccagtgcacagtttggatattactttcgtgtaacctgtaaggaagaaaaagtccttcgtaacaataaaaactttagtactgtagatatccagaagaatggtgttaaatttaccaacagcaaattgacttctttaaatgaagagtataccaaaaataaaacagaatatgaagaagcccaggatgccattgttaaagaaattgtcaatatttcttcaggctatgtagaaccaatgcagacactcaatgatgtgttagctcagctagatgctgttgtcagctttgctcacgtgtcaaatggagcacctgttccatatgtacgaccagccattttggagaaaggacaaggaagaattatattaaaagcatccaggcatgcttgtgttgaagttcaagatgaaattgcatttattcctaatgacgtatactttgaaaaagataaacagatgttccacatcattactggccccaatatgggaggtaaatcaacatatattcgacaaactggggtgatagtactcatggcccaaattgggtgttttgtgccatgtgagtcagcagaagtgtccattgtggactgcatcttagcccgagtaggggctggtgacagtcaattgaaaggagtctccacgttcatggctgaaatgttggaaactgcttctatcctcaggtctgcaaccaaagattcattaataatcatagatgaattgggaagaggaacttctacctacgatggatttgggttagcatgggctatatcagaatacattgcaacaaagattggtgctttttgcatgtttgcaacccattttcatgaacttactgccttggccaatcagataccaactgttaataatctacatgtcacagcactcaccactgaagagaccttaactatgctttatcaggtgaagaaaggtgtctgtgatcaaagttttgggattcatgttgcagagcttgctaatttccctaagcatgtaatagagtgtgctaaacagaaagccctggaacttgaggagtttcagtatattggagaatcgcaaggatatgatatcatggaaccagcagcaaagaagtgctatctggaaagagagcaaggtgaaaaaattattcaggagttcctgtccaaggtgaaacaaatgccctttactgaaatgtcagaagaaaacatcacaataaagttaaaacagctaaaagctgaagtaatagcaaagaataatagctttgtaaatgaaatcatttcacgaataaaagttactacgtgaaaaatcccagtaatggaatgaaggtaatattgataagctattgtctgtaatagttttatattgttttatattaaccctttttccatagtgttaactgtcagtgcccatgggctatcaacttaataagatatttagtaatattttactttgaggacattttcaaagatttttattttgaaaaatgagagctgtaactgaggactgtttgcaattgacataggcaataataagtgatgtgctgaattttataaataaaatcatgtagtttgtgg

SEQ ID NO: 59 - DNA, 4435 bp, Homo sapiens
ggcgaggcgcctgttgattggccactggggcccgggttcctccggcggagcgcgcctccccccagatttcccgccagcaggagccgcgcggtagatgcggtgcttttaggagctccgtccgacagaacggttgggccttgccggctgtcggtatgtcgcgacagagcaccctgtacagcttcttccccaagtctccggcgctgagtgatgccaacaaggcctcggccagggcctcacgcgaaggcggccgtgccgccgctgcccccggggcctctccttccccaggcggggatgcggcctggagcgaggctgggcctgggcccaggcccttggcgcgctccgcgtcaccgcccaaggcgaagaacctcaacggagggctgcggagatcggtagcgcctgctgcccccaccagttgtgacttctcaccaggagatttggtttgggccaagatggagggttacccctggtggccttgtctggtttacaaccacccctttgatggaacattcatccgcgagaaagggaaatcagtccgtgttcatgtacagttttttgatgacagcccaacaaggggctgggttagcaaaaggcttttaaagccatatacaggttcaaaatcaaaggaagcccagaagggaggtcatttttacagtgcaaagcctgaaatactgagagcaatgcaacgtgcagatgaagccttaaataaagacaagattaagaggcttgaattggcagtttgtgatgagccctcagagccagaagaggaagaagagatggaggtaggcacaacttacgtaacagataagagtgaagaagataatgaaattgagagtgaagaggaagtacagcctaagacacaaggatctaggcgaagtagccgccaaataaaaaaacgaagggtcatatcagattctgagagtgacattggtggctctgatgtggaatttaagccagacactaaggaggaaggaagcagtgatgaaataagcagtggagtgggggatagtgagagtgaaggcctgaacagccctgtcaaagttgctcgaaagcggaagagaatggtgactggaaatggctctcttaaaaggaaaagctctaggaaggaaacgccctcagccaccaaacaagcaactagcatttcatcagaaaccaagaatactttgagagctttctctgcccctcaaaattctgaatcccaagcccacgttagtggaggtggtgatgacagtagtcgccctactgtttggtatcatgaaactttagaatggcttaaggaggaaaagagaagagatgagcacaggaggaggcctgatcaccccgattttgatgcatctacactctatgtgcctgaggatttcctcaattcttgtactcctgggatgaggaagtggtggcagattaagtctcagaactttgatcttgtcatctgttacaaggtggggaaattttatgagctgtaccacatggatgctcttattggagtcagtgaactggggctggtattcatgaaaggcaactgggcccattctggctttcctgaaattgcatttggccgttattcagattccctggtgcagaagggctataaagtagcacgagtggaacagactgagactccagaaatgatggaggcacgatgtagaaagatggcacatatatccaagtatgatagagtggtgaggagggagatctgtaggatcattaccaagggtacacagacttacagtgtgctggaaggtgatccctctgagaactacagtaagtatcttcttagcctcaaagaaaaagaggaagattcttctggccatactcgtgcatatggtgtgtgctttgttgatacttcactgggaaagtttttcataggtcagttttcagatgatcgccattgttcgagatttaggactctagtggcacactatcccccagtacaagttttatttgaaaaaggaaatctctcaaaggaaactaaaacaattctaaagagttcattgtcctgttctcttcaggaaggtctgatacccggctcccagttttgggatgcatccaaaactttgagaactctccttgaggaagaatattttagggaaaagctaagtgatggcattggggtgatgttaccccaggtgcttaaaggtatgacttcagagtctgattccattgggttgacaccaggagagaaaagtgaattggccctctctgctctaggtggttgtgtcttctacctcaaaaaatgccttattgatcaggagcttttatcaatggctaattttgaagaatatattcccttggattctgacacagtcagcactacaagatctggtgctatcttcaccaaagcctatcaacgaatggtgctagatgcagtgacattaaacaacttggagatttttctgaatggaacaaatggttctactgaaggaaccctactagagagggttgatacttgccatactccttttggtaagcggctcctaaagcaatggctttgtgccccactctgtaaccattatgctattaatgatcgtctagatgccatagaagacctcatggttgtgcctgacaaaatctccgaagttgtagagcttctaaagaagcttccagatcttgagaggctactcagtaaaattcataatgttgggtctcccctgaagagtcagaaccacccagacagcagggctataatgtatgaagaaactacatacagcaagaagaagattattgattttctttctgctctggaaggattcaaagtaatgtgtaaaattatagggatcatggaagaagttgctgatggttttaagtctaaaatccttaagcaggtcatctctctgcagacaaaaaatcctgaaggtcgttttcctgatttgactgtagaattgaaccgatgggatacagcctttgaccatgaaaaggctcgaaagactggacttattactcccaaagcaggctttgactctgattatgaccaagctcttgctgacataagagaaaatgaacagagcctcctggaatacctagagaaacagcgcaacagaattggctgtaggaccatagtctattgggggattggtaggaaccgttaccagctggaaattcctgagaatttcaccactcgcaatttgccagaagaatacgagttgaaatctaccaagaagggctgtaaacgatactggaccaaaactattgaaaagaagttggctaatctcataaatgctgaagaacggagggatgtatcattgaaggactgcatgcggcgactgttctataactttgataaaaattacaaggactggcagtctgctgtagagtgtatcgcagtgttggatgttttactgtgcctggctaactatagtcgagggggtgatggtcctatgtgtcgcccagtaattctgttgccggaagataccccccccttcttagagcttaaaggatcacgccatccttgcattacgaagactttttttggagatgattttattcctaatgacattctaataggctgtgaggaagaggagcaggaaaatggcaaagcctattgtgtgcttgttactggaccaaatatggggggcaagtctacgcttatgagacaggctggcttattagctgtaatggcccagatgggttgttacgtccctgctgaagtgtgcaggctcacaccaattgatagagtgtttactagacttggtgcctcagacagaataatgtcaggtgaaagtacattttttgttgaattaagtgaaactgccagcatactcatgcatgcaacagcacattctctggtgcttgtggatgaattaggaagaggtactgcaacatttgatgggacggcaatagcaaatgcagttgttaaagaacttgctgagactataaaatgtcgtacattattttcaactcactaccattcattagtagaagattattctcaaaatgttgctgtgcgcctaggacatatggcatgcatggtagaaaatgaatgtgaagaccccagccaggagactattacgttcctctataaattcattaagggagcttgtcctaaaagctatggctttaatgcagcaaggcttgctaatctcccagaggaagttattcaaaagggacatagaaaagcaagagaatttgagaagatgaatcagtcactacgattatttcgggaagtttgcctggctagtgaaaggtcaactgtagatgctgaagctgtccataaattgctgactttgattaaggaattatagactgactacattggaagctttgagttgacttctgacaaaggtggtaaattcagacaacattatgatctaataaactttattttttaaaaatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 60 - DNA, 4472 bp, Homo sapiens
ccgcagacgcctgggaactgcggccgcgggctcgcgctcctcgccaggccctgccgccgggctgccatccttgccctgccatgtctcgccggaagcctgcgtcgggcggcctcgctgcctccagctcagcccctgcgaggcaagcggttttgagccgattcttccagtctacgggaagcctgaaatccacctcctcctccacaggtgcagccgaccaggtggaccctggcgctgcagcggctgcagcggccgcagcggccgcagcgcccccagcgcccccagctcccgccttcccgccccagctgccgccgcacatagctacagaaattgacagaagaaagaagagaccattggaaaatgatgggcctgttaaaaagaaagtaaagaaagtccaacaaaaggaaggaggaagtgatctgggaatgtctggcaactctgagccaaagaaatgtctgaggaccaggaatgtttcaaagtctctggaaaaattgaaagaattctgctgcgattctgcccttcctcaaagtagagtccagacagaatctctgcaggagagatttgcagttctgccaaaatgtactgattttgatgatatcagtcttctacacgcaaagaatgcagtttcttctgaagattcgaaacgtcaaattaatcaaaaggacacaacactttttgatctcagtcagtttggatcatcaaatacaagtcatgaaaatttacagaaaactgcttccaaatcagctaacaaacggtccaaaagcatctatacgccgctagaattacaatacatagaaatgaagcagcagcacaaagatgcagttttgtgtgtggaatgtggatataagtatagattctttggggaagatgcagagattgcagcccgagagctcaatatttattgccatttagatcacaactttatgacagcaagtatacctactcacagactgtttgttcatgtacgccgcctggtggcaaaaggatataaggtgggagttgtgaagcaaactgaaactgcagcattaaaggccattggagacaacagaagttcactcttttcccggaaattgactgccctttatacaaaatctacacttattggagaagatgtgaatcccctaatcaagctggatgatgctgtaaatgttgatgagataatgactgatacttctaccagctatcttctgtgcatctctgaaaataaggaaaatgttagggacaaaaaaaagggcaacatttttattggcattgtgggagtgcagcctgccacaggcgaggttgtgtttgatagtttccaggactctgcttctcgttcagagctagaaacccggatgtcaagcctgcagccagtagagctgctgcttccttcggccttgtccgagcaaacagaggcgctcatccacagagccacatctgttagtgtgcaggatgacagaattcgagtcgaaaggatggataacatttattttgaatacagccatgctttccaggcagttacagagttttatgcaaaagatacagttgacatcaaaggttctcaaattatttctggcattgttaacttagagaagcctgtgatttgctctttggctgccatcataaaatacctcaaagaattcaacttggaaaagatgctctccaaacctgagaattttaaacagctatcaagtaaaatggaatttatgacaattaatggaacaacattaaggaatctggaaatcctacagaatcagactgatatgaaaaccaaaggaagtttgctgtgggttttagaccacactaaaacttcatttgggagacggaagttaaagaagtgggtgacccagccactccttaaattaagggaaataaatgcccggcttgatgctgtatcggaagttctccattcagaatctagtgtgtttggtcagatagaaaatcatctacgtaaattgcccgacatagagaggggactctgtagcatttatcacaaaaaatgttctacccaagagttcttcttgattgtcaaaactttatatcacctaaagtcagaatttcaagcaataatacctgctgttaattcccacattcagtcagacttgctccggaccgttattttagaaattcctgaactcctcagtccagtggagcattacttaaagatactcaatgaacaagctgccaaagttggggataaaactgaattatttaaagacctttctgacttccctttaataaaaaagaggaaggatgaaattcaaggtgttattgacgagatccgaatgcatttgcaagaaatacgaaaaatactaaaaaatccttctgcacaatatgtgacagtatcaggacaggagtttatgatagaaataaagaactctgctgtatcttgtataccaactgattgggtaaaggttggaagcacaaaagctgtgagccgctttcactctccttttattgtagaaaattacagacatctgaatcagctccgggagcagctagtccttgactgcagtgctgaatggcttgattttctagagaaattcagtgaacattatcactccttgtgtaaagcagtgcatcacctagcaactgttgactgcattttctccctggccaaggtcgctaagcaaggagattactgcagaccaactgtacaagaagaaagaaaaattgtaataaaaaatggaaggcaccctgtgattgatgtgttgctgggagaacaggatcaatatgtcccaaataatacagatttatcagaggactcagagagagtaatgataattaccggaccaaacatgggtggaaagagctcctacataaaacaagttgcattgattaccatcatggctcagattggctcctatgttcctgcagaagaagcgacaattgggattgtggatggcattttcacaaggatgggtgctgcagacaatatatataaaggacagagtacatttatggaagaactgactgacacagcagaaataatcagaaaagcaacatcacagtccttggttatcttggatgaactaggaagagggacgagcactcatgatggaattgccattgcctatgctacacttgagtatttcatcagagatgtgaaatccttaaccctgtttgtcacccattatccgccagtttgtgaactagaaaaaaattactcacaccaggtggggaattaccacatgggattcttggtcagtgaggatgaaagcaaactggatccaggcgcagcagaacaagtccctgattttgtcaccttcctttaccaaataactagaggaattgcagcaaggagttatggattaaatgtggctaaactagcagatgttcctggagaaattttgaagaaagcagctcacaagtcaaaagagctggaaggattaataaatacgaaaagaaagagactcaagtattttgcaaagttatggacgatgcataatgcacaagacctgcagaagtggacagaggagttcaacatggaagaaacacagacttctcttcttcattaaaatgaagactacatttgtgaacaaaaaatggagaattaaaaataccaactgtacaaaataactctccagtaacagcctatctttgtgtgacatgtgagcataaaattatgaccatggtatattcctattggaaacagagaggtttttctgaagacagtctttttcaagtttctgtcttcctaacttttctacgtataaacactcttgaatagacttccactttgtaattagaaaattttatggacagtaagtccagtaaagccttaagtggcagaatataattcccaagcttttggagggtgatataaaaatttacttgatatttttatttgtttcagttcagataattggcaactgggtgaatctggcaggaatctatccattgaactaaaataattttattatgcaaccagtttatccaccaagaacataagaattttttataagtagaaagaattggccaggcatggtggctcatgcctgtaatcccagcactttgggaggccaaggtaggcagatcacctgaggtcaggagttcaagaccagcctggccaacatggcaaaaccccatctttactaaaaatataaagtacatctctactaaaaatacgaaaaaattagctgggcatggtggcgcacacctgtagtcccagctactccggaggctgaggcaggagaatctcttgaacctgggaggcggaggttgcaatgagccgagatcacgtcactgcactccagcttgggcaacagagcaagactccatctcaaaaaaaaaaaaagaaaaaagaaaagaaatagaattatcaagcttttaaaaactagagcacagaaggaataaggtcatgaaatttaaaaggttaaatattgtcataggattaagcagtttaaagattgttggatgaaattatttgtcattcattcaagtaataaatatttaatgaatacttgctataaaaaaaaaaaaaaaaaaaaaaaaaa

SEQ ID NO: 61 - DNA, 3538 bp, Homo sapiens
ggcaagacaacgaggatttgcgtagggggcgagcctctgaggccacttggctcttacggccacgcagggcgccgcagatgcagccggagcccgcttttccctctcaggacgacccctaggccgccagcagttccctaccgacgaaggcgactgtacagcgtccaccgcgttcgtgcccacttacccgccgccccactccgggccgccggctcgcagcaggaccagcccggctgctacggccgcggatacacgccctcaggcccggcgctgcgcagcttgcggaagctttcccggacagactcgctgccagcggattggctgcgagcagcgccaatctcacgttgcccccgggcgaggcgggactcagtgccgcgctctctgcacccgctctgccgcgcgcgtgcgtgctgggtgcgggtgcgggtgcggggttgggcctgcgcatcgggtgagacgctggctgcttgcggctagtggatggtaattgcctgcctcgcgctagcaggaagctgctctgttaaaagcgaaaatgaaacaattgcctgcggcaacagttcgactcctttcaagttctcagatcatcacttcggtggtcagtgttgtaaaagagcttattgaaaactccttggatgctggtgccacaagcgtagatgttaaactggagaactatggatttgataaaattgaggtgcgagataacggggagggtatcaaggctgttgatgcacctgtaatggcaatgaagtactacacctcaaaaataaatagtcatgaagatcttgaaaatttgacaacttacggttttcgtggagaagccttggggtcaatttgttgtatagctgaggttttaattacaacaagaacggctgctgataattttagcacccagtatgttttagatggcagtggccacatactttctcagaaaccttcacatcttggtcaaggtacaactgtaactgctttaagattatttaagaatctacctgtaagaaagcagttttactcaactgcaaaaaaatgtaaagatgaaataaaaaagatccaagatctcctcatgagctttggtatccttaaacctgacttaaggattgtctttgtacataacaaggcagttatttggcagaaaagcagagtatcagatcacaagatggctctcatgtcagttctggggactgctgttatgaacaatatggaatcctttcagtaccactctgaagaatctcagatttatctcagtggatttcttccaaagtgtgatgcagaccactctttcactagtctttcaacaccagaaagaagtttcatcttcataaacagtcgaccagtacatcaaaaagatatcttaaagttaatccgacatcattacaatctgaaatgcctaaaggaatctactcgtttgtatcctgttttctttctgaaaatcgatgttcctacagctgatgttgatgtaaatttaacaccagataaaagccaagtattattacaaaataaggaatctgttttaattgctcttgaaaatctgatgacgacttgttatggaccattacctagtacaaattcttatgaaaataataaaacagatgtttccgcagctgacatcgttcttagtaaaacagcagaaacagatgtgctttttaataaagtggaatcatctggaaagaattattcaaatgttgatacttcagtcattccattccaaaatgatatgcataatgatgaatctggaaaaaacactgatgattgtttaaatcaccagataagtattggtgactttggttatggtcattgtagtagtgaaatttctaacattgataaaaacactaagaatgcatttcaggacatttcaatgagtaatgtatcatgggagaactctcagacggaatatagtaaaacttgttttataagttccgttaagcacacccagtcagaaaatggcaataaagaccatatagatgagagtggggaaaatgaggaagaagcaggtcttgaaaactcttcggaaatttctgcagatgagtggagcaggggaaatatacttaaaaattcagtgggagagaatattgaacctgtgaaaattttagtgcctgaaaaaagtttaccatgtaaagtaagtaataataattatccaatccctgaacaaatgaatcttaatgaagattcatgtaacaaaaaatcaaatgtaatagataataaatctggaaaagttacagcttatgatttacttagcaatcgagtaatcaagaaacccatgtcagcaagtgctctttttgttcaagatcatcgtcctcagtttctcatagaaaatcctaagactagtttagaggatgcaacactacaaattgaagaactgtggaagacattgagtgaagaggaaaaactgaaatatgaagagaaggctactaaagacttggaacgatacaatagtcaaatgaagagagccattgaacaggagtcacaaatgtcactaaaagatggcagaaaaaagataaaacccaccagcgcatggaatttggcccagaagcacaagttaaaaacctcattatctaatcaaccaaaacttgatgaactccttcagtcccaaattgaaaaaagaaggagtcaaaatattaaaatggtacagatccccttttctatgaaaaacttaaaaataaattttaagaaacaaaacaaagttgacttagaagagaaggatgaaccttgcttgatccacaatctcaggtttcctgatgcatggctaatgacatccaaaacagaggtaatgttattaaatccatatagagtagaagaagccctgctatttaaaagacttcttgagaatcataaacttcctgcagagccactggaaaagccaattatgttaacagagagtctttttaatggatctcattatttagacgttttatataaaatgacagcagatgaccaaagatacagtggatcaacttacctgtctgatcctcgtcttacagcgaatggtttcaagataaaattgataccaggagtttcaattactgaaaattacttggaaatagaaggaatggctaattgtctcccattctatggagtagcagatttaaaagaaattcttaatgctatattaaacagaaatgcaaaggaagtttatgaatgtagacctcgcaaagtgataagttatttagagggagaagcagtgcgtctatccagacaattacccatgtacttatcaaaagaggacatccaagacattatctacagaatgaagcaccagtttggaaatgaaattaaagagtgtgttcatggtcgcccattttttcatcatttaacctatcttccagaaactacatgattaaatatgtttaagaagattagttaccattgaaattggttctgtcataaaacagcatgagtctggttttaaattatctttgtattatgtgtcacatggttattttttaaatgaggattcactgacttgtttttatattgaaaaaagttccacgtattgtagaaaacgtaaataaactaatatagactattcaaaaaaaaaaaaaaaaaa

SEQ ID NO: 62 - DNA, 7911 bp, Homo sapiens
aacaactggtgcgcatgcgcactggtgtctcgcggcctggcgcgccccctccgaagcgcatgctcgtgggcacgcacgagcctcaagatccaaggtgcgcgcgtcggcgtccgaggcggttggtgtcggagaatttgttaagcgggactccaggcaattatttccagtcagagaaggaaaccagtgcctggcattctcaccatctttctacctaccatgatcaagtgcttgtcagttgaagtacaagccaaattgcgttctggtttggccataagctccttgggccaatgtgttgaggaacttgccctcaacagtattgatgctgaagcaaaatgtgtggctgtcagggtgaatatggaaaccttccaagttcaagtgatagacaatggatttgggatggggagtgatgatgtagagaaagtgggaaatcgttatttcaccagtaaatgccactcggtacaggacttggagaatccaaggttttatggtttccgaggagaggccttggcaaatattgctgacatggccagtgctgtggaaatttcgtccaagaaaaacaggacaatgaaaacttttgtgaaactgtttcagagtggaaaagccctgaaagcttgtgaagctgatgtgactagagcaagcgctgggactactgtaacagtgtataacctattttaccagcttcctgtaaggaggaaatgcatggaccctagactggagtttgagaaggttaggcagagaatagaagctctctcactcatgcacccttccatttctttctctttgagaaatgatgtttctggttccatggttcttcagctccctaaaaccaaagacgtatgttcccgattttgtcaaatttatggattgggaaagtcccaaaagctaagagaaataagttttaaatataaagagtttgagcttagtggctatatcagctctgaagcacattacaacaagaatatgcagtttttgtttgtgaacaaaagactagttttaaggacaaagctacataaactcattgactttttattaaggaaagaaagtattatatgcaagccaaagaatggtcccaccagtaggcaaatgaattcaagtcttcggcaccggtctaccccagaactctatggcatatatgtaattaatgtgcagtgccaattctgtgagtatgatgtgtgcatggagccagccaaaactctgattgaatttcagaactgggacactctcttgttttgcattcaggaaggagtgaaaatgtttttaaagcaagaaaaattatttgtggaattatcaggtgaggatattaaggaatttagtgaagataatggttttagtttatttgatgctactcttcagaagcgtgtgacttccgatgagaggagcaatttccaggaagcatgtaataatattttagattcctatgagatgtttaatttgcagtcaaaagctgtgaaaagaaaaactactgcagaaaacgtaaacacacagagttctagggattcagaagctaccagaaaaaatacaaatgatgcatttttgtacatttatgaatcaggtggtccaggccatagcaaaatgacagagccatctttacaaaacaaagacagctcttgctcagaatcaaagatgttagaacaagagacaattgtagcatcagaagctggagaaaatgagaaacataaaaaatctttcctggaacatagctctttagaaaatccgtgtggaaccagtttagaaatgtttttaagcccttttcagacaccatgtcactttgaggagagtgggcaggatctagaaatatggaaagaaagtactactgttaatggcatggctgccaacatcttgaaaaataatagaattcagaatcaaccaaagagatttaaagatgctactgaagtgggatgccagcctctgccttttgcaacaacattatggggagtacatagtgctcagacagagaaagagaaaaaaaaagaatctagcaattgtggaagaagaaatgtttttagttatgggcgagttaaattatgttccactggctttataactcatgtagtacaaaatgaaaaaactaaatcaactgaaacagaacattcatttaaaaattatgttagacctggtcccacacgtgcccaagaaacatttggaaatagaacacgtcattcagttgaaactccagacatcaaagatttagccagcactttaagtaaagaatctggtcaattgcccaacaaaaaaaattgcagaacgaatataagttatgggctagagaatgaacctacagcaacttatacaatgttttctgcttttcaggaaggtagcaaaaaatcacaaacagattgcatattatctgatacatccccctctttcccctggtatagacacgtttccaatgatagtaggaaaacagataaattaattggtttctccaaaccaatcgtccgtaagaagctaagcttgagttcacagctaggatctttagagaagtttaagaggcaatatgggaaggttgaaaatcctctggatacagaagtagaggaaagtaatggagtcactaccaatctcagtcttcaagttgaacctgacattctgctgaaggacaagaaccgcttagagaactctgatgtttgtaaaatcactactatggagcatagtgattcagatagtagttgtcaaccagcaagccacatccttaactcagagaagtttccattctccaaggatgaagattgtttagaacaacagatgcctagtttgagagaaagtcctatgaccctgaaggagttatctctctttaatagaaaacctttggaccttgagaagtcatctgaatcactagcctctaaattatccagactgaagggttccgaaagagaaactcaaacaatggggatgatgagtcgttttaatgaacttccaaattcagattccagtaggaaagacagcaagttgtgcagtgtgttaacacaagatttttgtatgttatttaacaacaagcatgaaaaaacagagaatggtgtcatcccaacatcagattctgccacacaggataattcctttaataaaaatagtaaaacacattctaacagcaatacaacagagaactgtgtgatatcagaaactcctttggtattgccctataataattctaaagttaccggtaaagattcagatgttcttatcagagcctcagaacaacagataggaagtcttgactctcccagtggaatgttaatgaatccggtagaagatgccacaggtgaccaaaatggaatttgttttcagagtgaggaatctaaagcaagagcttgttctgaaactgaagagtcaaacacgtgttgttcagattggcagcggcatttcgatgtagccctgggaagaatggtttatgtcaacaaaatgactggactcagcacattcattgccccaactgaggacattcaggctgcttgtactaaagacctgacaactgtggctgtggatgttgtacttgagaatgggtctcagtacaggtgtcaaccttttagaagcgaccttgttcttcctttccttccgagagctcgagcagagaggactgtgatgagacaggataacagagatactgtggatgatactgttagtagcgaatcgcttcagtctttgttctcagaatgggacaatccagtatttgcccgttatccagaggttgctgttgatgtaagcagtggccaggctgagagcttagcagttaaaattcacaacatcttgtatccctatcgtttcaccaaaggaatgattcattcaatgcaggttctccagcaagtagataacaagtttattgcctgtttgatgagcactaagactgaagagaatggcgaggcaggtgggaacctgctcgtgctggtggatcagcacgctgcccatgagcgtatacgtctggagcagcttatcattgattcctacgagaagcaacaggcacaaggctctggtcggaaaaaattactgtcttctactctaattcctccgctagagataacagtgacagaggaacaaaggagactcttatggtgttaccacaaaaatctggaagatctgggccttgaatttgtatttccagacactagtgattctctggtccttgtgggaaaagtaccactatgttttgtggaaagagaagccaatgaacttcggagaggaagatctactgtgaccaagagtattgtggaggaatttatccgagaacaactggagctactccagaccaccggaggcatccaagggacattgccactgactgtccagaaggtgttggcatcccaagcctgccatggggccattaagtttaatgatggcctgagcttacaggaaagttgccgccttattgaagctctgtcctcatgccagctgccattccagtgtgctcacgggagaccttctatgctgccgttagctgacatagaccacttggaacaggaaaaacagattaaacccaacctcactaaacttcgcaaaatggcccaggcctggcgtctctttggaaaagcagagtgtgatacaaggcagagcctgcagcaatccatgcctccctgtgagccaccatgagaacagaatcactggtctaaaaggaacaaagggatgttcactgtatgcctctgagcagagagcagcagcagcaggtaccagcacggccctgactgaatcagcccagtgtccctgagcagcttagacagcagggctctctgtatcagtctttcttgagcagatgattcccctagttgagtagccagatgaaattcaagcctaaagacaattcattcatttgcatccatgggcacagaaggttgctatatagtatctaccttttgctacttatttaatgataaaatttaatgacagtttgattggttgcttggtttgttatttgaagggtgtgatttttgtttttgtacagttttttttcaagcttcacatttgcgtgtatctaattcagctgatgctcaagtccaaggggtagtctgccttcccaggctgcccccagggtttctgcactggtcccctcttttcccttcagtcttcttcacttccctatgctgctgcttcatgtgctacatctcagacttaaagagtttctctactacagtgaaaacattctctagggtctttcatcaggcctttagttattttagggataaaaactattgataaaaaggacaaggatagaacagagaaaatttaaagtcctgttccgggttttttgttatgttttctttaaaaactcagagactgatgttcaatatcccaaaccagtaaaatggtgaaaatactatgagcttgttttttaaaatatgattttttttggtactttataaagtatctctttatgtgaaagcaattgtcatatcaaaacacagcatacatacgttcaacctaaccaaatatctttacactttttctttcaggagacaagggttctttgggtccctttcaaacggtatcttggtgttattacattatgcctatctattgcccttataatatcacttgggaccaggactgatcgttctgcaaatgcttgttatgccattctcaatctatttttcccgcaccttttcacatgatttgtggttaataggactcaacagactaaaattgcatagtagaaaaaaaatgcaaaaagccagctggtaatgtttattgcaactggggtgctatacaattagtaagatgatgcaatgagaatttctacttttgtatttcctgaccagcctgctcaaagtggcttttatatcaattgaatgattttcctcattttttaatacaggaaaccaattcgtgctcatggaagaaaagttcctttgccagcagccttgaagtgaatcttacaggagcaatgaaagtattgcattcattagcgtctgccccagagaaggttcagagaaaaccttcacttgttttcaaggggatccttgtagatttacgtaattggaatcctgaagaacaggccctactgtctaaaaaatggcttttattcttctaaatacatataaacggatgttttatagatgggaagacatgaccttagaaaggagagagttttcagaggatttgccaggctgtcaggggctctgcctccaggcccagtgtggcagtgtggcctcagggcctccgcctccctgcttgagggctgcatggaggccaactgtcctgggagttgtaaaaatcttttaaggccagaccaatttgagggattttaaaaagtgtctcagtgcctcttatgatttcagaaggttttgctatatgtaatcccaactactgttttcttgagagtagcagaggattagaaaaagtcctccataaattatgtaaccggccttcctgactagcctgactcaagcaatgtaagagataattattctgttttcataatttataagtgtgggggcatgcctcagcataaaaacaacctattagggaaaaatatctaatagattacctttatcgcctgttagggttttatgttgtttttaactcagatgccataagaacaaagatacatgtaatttataatagtaatcattaatacctatattgtgctttaaggtttacaaaataatttttctcatactttatcttagtttagtttcttgacagtccatgaggtaaggtggtagctttatcaccattttacaaagtgggaaacgaaggttcctcttaggaacctagttgtcacctttgtataataaaacttcgaagctcggagctgttaactggtttgctgaaggcttagctgtaagagccagaattcagacccaggtctgagtgacttcaaactgcacagtccttcccattattacccatatgctatcccttatatttttaatttattaggaattcattcatttataaacttggtgattcacctttattagattctggtcgctgaaggctttagtaacttcagagtaaaacttgagagatgagatgtaaaatgcagccattcttgagagttcctttttctgtaacattcatcaacacttcattgagaagtgaaggttcctatggctgtctctaccttcaagaggcttagctttagtcactgagaaagacaaggaaactaatgatagaatatagtagcttcttctggcgttaggtatcacagagtcacagctagttacagctagccctttattattgaaagaagaggagctagcagtcccactatcagaattaagactagagatggtaataggagctagtatcagaaaagcttaaggcaaagcataaagtgtaggctagaatgaagctggagaatggggagggggcttgggtaacatccagaacctggctggggacctggaactacatgagatgtaagaatggagaggttctagcagtcagaggtcaggtacaaatgaacagctgggatctgcgcatggcagacagtgaaaaaacccaggcaagcaaaatggtcagagcagaaaggggcccaaggccacgttcttgagatgtggagggggctgaggaagccacgccaagtaaggacagatgcagctcagcagttcctagcgagccctgacaagccagctcagctgaagcttcgggtgggagccagtcatggcacagtggagtgaaggaagagcagtttcaggcacccaaaacctgacccccacgacctgttttccacctgaagagccacccattccatccaaacccttggcaaaagtctgctaacagagagaaccggccagtatgctggccagtcgcgatcatgcctgtctttaccctctaagctgaagctgctcatcaacggtgagatggcaaaaaggtgggtccagaagaggggaaaagaagggagtctgtgaaaacaaaatgctgaagaatctgcatcaaataaacccttccttccttcctttttccttcaaaaaaaaaaaaaaa

SEQ ID NO: 63 - DNA, 20 bp, Artificial Sequence
  A synthetic oligonucleotide
gtgtaggaggggagcattga

SEQ ID NO: 64 - DNA, 30 bp, Artificial Sequence
  A synthetic oligonucleotide
catgaagacctcacagtaaaaataggtgat